(19) United States
(12) Patent Application Publication
(10) Pub. No.: US 2006/0241134 A1
(43) Pub. Date: Oct. 26, 2006

(54) Pharmaceutical combinations of a proton pump inhibitor and a compound which modifies gastrointestinal motility

(75) Inventors: Wilm Buhr, Konstanz (DE); Stefan Postius, Konstanz (DE)

Correspondence Address: NATH & ASSOCIATES PLLC, 112 South West Street, Alexandria, VA, 22314, US

(73) Assignee: Altana Pharma GA, Konstanz (DE)

(21) Appl. No.: 10/557,414

(22) PCT Filed: May 26, 2004
(86) PCT No.: PCT/EP04/50936

§ 371 Date: Nov. 18, 2005

(30) Foreign Application Priority Data
May 27, 2003 (EP) 03011875.6
May 25, 2004 (EP) 04102304.5

(51) Int. Cl. A61K 31/4745

(52) U.S. Cl. 514/291

(57) ABSTRACT

The invention relates to the combination of certain active compounds from the acid pump antagonist class and compounds which modify gastrointestinal motility.

### FIELD OF APPLICATION OF THE INVENTION

The invention relates to the combination of certain active compounds for therapeutic purposes. The substances used in the combination according to the present invention are known active compounds from the acid pump antagonist class and compounds, which modify gastrointestinal motility, or compounds, which reduce the incidence of transient lower esophageal sphincter relaxation (TLOSR).

### KNOWN TECHNICAL BACKGROUND

A number of compounds, which inhibit gastric acid secretion by reversible blockade of the proton pump, are known from prior art. These compounds are termed as reversible proton pump inhibitors or, latterly, as acid pump antagonists. The use of these compounds in the treatment of gastrointestinal diseases, gastrointestinal inflammatory diseases and/or the gastro-esophageal reflux disease (GERD) is also described in the prior art.

Further on, the prior art discloses compounds, which modify gastrointestinal motility by different ways. Thus, for example, the international applications WO 02100823, WO 02100869, WO 02100870 and WO 02100871 disclose compounds, which reduce the incidence of transient lower esophageal sphincter relaxation (TLOSR). Said International applications are incorporated by reference into the specification of the present invention in their entirety for all purposes.

Still further, the prior art teaches the utilizability of compounds, which modify gastrointestinal motility by any way, for therapy of miscellaneous gastrointestinal diseases.

The international application WO 0069438 discloses, Inter alia, pharmaceutical compositions comprising NK-1 anatgonists and proton pump inhibitors exemplified by omeprazole, lansoprazole, pantoprazole, leminoprazole and certain salts of the (−)-enantiomer of omeprazole, which are said to be useful in the prevention and treatment of diseases brought about by hypersecretion of gastric acid in the gut and/or relaxation of the lower esophageal sphincter.

The international application WO 0185167 discloses pharmaceutical compositions comprising gastrin/cholecystokinin receptor ligands and certain proton pump inhibitors exemplified inter alia by (RS)-rabeprazole, (RS)-omeprazole, lansoprazole, pantoprazole, (R)-omeprazole, (S)-omeprazole, perprazole, (R)-rabeprazole, (S)-rabeprazole, or the alkaline salts thereof, which are said to be useful to reduce hyperplasia, associated with administration of proton pump inhibitors.

The international application WO 0141748 discloses pharmaceutical combinations comprising a 5-HT4 partial agonist or a 5-HT4 antagonist, and, inter alia, a reversible proton pump inhibitor and their uses in treating gastrointestinal disorders; Reversible proton pump inhibitors mentioned therein are exemplified inter alia by pumaprazole, SKF 97574, SKF 96067, H 40502, YH1238 and YH1885.

The U.S. patent U.S. Pat. No. 6,552,045 describes pharmaceutical combinations which act at three different sites: action at 5-HT3 receptors, 5-HT4 receptors and either H2 receptors or proton pumps; Proton pump inhibitors disclosed therein are exemplified inter alia by prazole derivatives.

The international application WO2004/000855 describes medicaments comprising an acid secretion inhibiting agent and a reflux inhibitor which inhibits transient esophageal sphincter relaxations. As an acid secretion inhibiting agent, inter alia, reversible and irreversible proton pump inhibitors are mentioned generally, whereby certain prazole derivatives are mentioned exemplarily.

The international application WO2004/000856 describes medicaments comprising a bicyclic imidazopyridine compound and a reflux inhibitor which inhibits transient esophageal sphincter relaxations. The US application US20040092511 discloses pharmaceutical combinations comprising an agent selected from the group consisting of 5-HT4 partial agonists, 5-HT4 agonists or antagonists, and 5-HT3 antagonists, and, inter alia, a reversible proton pump inhibitor and their uses in treating gastrointestinal disorders; Reversible proton pump inhibitors mentioned therein are exemplified inter alia by pumaprazole, SKF 97574, SKF 96067, H 40502, BY 112, YH1238 and YH1885.

The document K. Fujimori et al., Allergology International, Blackwell Science, vol. 46, no. 3, 1997, p. 167-172 describes combined omeprazole and cisapride treatment in asthmatics with reflux esophagitis.

The document A. R. Soylu et al., Gastroenterology, Saunders, vol. 120, no. 5, 2001, p. A-403 describes combined lansoprazole and cisapride therapy of pulmonary symptoms in asthmatics with gastroesophageal reflux.

There is still a severe need in the art of having drug therapies of gastrointestinal diseases, advantageously of gastro-esophageal reflux disease (GERD) or irritable bowel syndrome (IBS).

Accordingly, there is a need to invent new combinations of active compounds that when used together show preferred therapeutic profiles and/or are more efficacious than when used alone.

The combinations per se and the combined use of certain active compounds purposively selected from the acid pump antagonist class and compounds, which modify gastrointestinal motility, and/or compounds, which reduce the incidence of transient lower esophageal sphincter relaxation (TLOSR), in the sense disclosed in this invention for therapeutic purposes has not yet been described in the prior art.

The present invention refers to combinations which are distinguishable from the prior art in their constituents, pharmacological action or activity, and/or therapeutical effectiveness or tolerance.

Notably and advantageously, in contrast to combinations described in the prior art comprising irreversible proton pump inhibitors (such e.g. prazole derivatives), the present invention refers to combinations comprising certain reversible proton pump inhibitors (i.e. acid pump antagonists).

### DESCRIPTION OF THE INVENTION

Surprisingly and unanticipatedly, it has now been found that certain, purposively selected acid pump antagonists are particularly useful and beneficial to be employed in functional and synergistic combination with compounds, which modify gastrointestinal motility, for precise therapy or prophylaxis of gastrointestinal diseases, in particular of gastro-esophageal reflux disease (GERD) or irritable bowel syndrome (IBS).

Accordingly, in one more detailed facet, it has also been found that those certain, purposively selected acid pump antagonists are particularly useful and beneficial to be employed in functional and synergistic combination with compounds, which reduce the incidence of transient lower esophaneal sphincter relaxation (TLOSR), for precise therapy or prophylaxis of gastrointestinal diseases, in particular of gastro-esophageal reflux disease (GERD).

The term “acid pump antagonists” refers to those compounds which inhibit by blockade of the proton pump the gastric acid secretion without binding covalently to the H⁺/K⁺-ATPase, the enzyme responsible for gastric acid secretion. Within the scope of this invention, the term “acid pump antagonists” comprises not only the active compounds per se but also pharmacologically acceptable salts, solvates (in particular hydrates) and solvates of the salts of these compounds.

Acid pump antagonists in the meaning of this invention can be from the class of imidazopyridines, such as, for example, those mentioned below.

Within the scope of this invention, the term “acid pump antagonists” refers in a first detail (detail a) of the present invention to tricyclic imidazo[1,2-a]pyridine compounds, which are selected from a group consisting of those tricyclic imidazo[1,2-a]pyridine compounds which are specifically disclosed and/or individualized and/or claimed in the following patent applications and patents:

WO 9842707, WO 0017200, WO 0026217, WO 0063211, WO 0172756, WO 0172755, WO 0172757, WO 0234749, WO 03014120, WO 03014123, WO 03016310 and WO 03091253;

and/or to those compounds which are mentioned expressis verbis in the List A below;

List A consists of the following compounds:
(7S,8R,9R)-2,3-dimethyl-7,8-dihydroxy-9-phenyl-7,8,9,10-tetrahydroimidazo[1,2-h][1,7]naphthyridine,
(7S,8R,9R)-7,8-isopropylidenedioxy-2,3-dimethyl-9-phenyl-7,8,9,10-tetrahydroimidazo[1,2-h][1,7]naphthyridine,
7,8-dihydroxy-9-phenyl-2,3-dimethyl-7H-8,9-dihydropyrano[2,3-c]imidazo[1,2-a]pyridine,
(7R,8R,9R)-2,3-dimethyl-8-hydroxy-7-methoxy-9-phenyl-7,8,9,10-tetrahydroimidazo[1,2-h][1,7]naphthyridine,
(7S,8S,9S)-2,3-dimethyl-8-hydroxy-7-methoxy-9-phenyl-7,8,9,10-tetrahydroimidazo[1,2-h][1,7]naphthyridine,
(7S,8R,9R)-2,3-dimethyl-8-hydroxy-7-methoxy-9-phenyl-7,8,9,10-tetrahydroimidazo[1,2-h][1,7]naphthyridine,
(7R,8S,9S)-2,3-dimethyl-8-hydroxy-7-methoxy-9-phenyl-7,8,9,10-tetrahydroimidazo[1,2-h][1,7]naphthyridine,
(7R,8R,9R)-2,3-dimethyl-7-ethoxy-8-hydroxy-9-phenyl-7,8,9,10-tetrahydroimidazo[1,2-h][1,7]naphthyridine,
(7S,8R,9R)-2,3-dimethyl-7-ethoxy-8-hydroxy-9-phenyl-7,8,9,10-tetrahydroimidazo[1,2-h][1,7]naphthyridine,
(7R,8R,9R)-2,3-dimethyl-8-hydroxy-7-(2-methoxyethoxy)-9-phenyl-7,8,9,10-tetrahydroimidazo-[1,2-h][1,7]naphthyridine,
(7S,8S,9S)-2,3-dimethyl-8-hydroxy-7-(2-methoxyethoxy)-9-phenyl-7,8,9,10-tetrahydroimidazo-[1,2-h][1,7]naphthyridine,
(7S,8R,9R)-2,3-dimethyl-8-hydroxy-7-(2-methoxyethoxy)-9-phenyl-7,8,9,10-tetrahydroimidazo-[1,2-h][1,7]naphthyridine,
(7R,8S,9S)-2,3-dimethyl-8-hydroxy-7-(2-methoxyethoxy)-9-phenyl-7,8,9,10-tetrahydroimidazo[1,2-h][1,7]naphthyridine,
(7S,8R,9R)-2,3-dimethyl-8-hydroxy-9-phenyl-7-(2-propoxy)-7,8,9,10-tetrahydroimidazo[1,2-h][1,7]-naphthyridine,
(7R,8R,9R)-2,3-dimethyl-7,8-dimethoxy-9-phenyl-7,8,9,10-tetrahydroimidazo[1,2-h][1,7]naphthyridine,
(7R,8R,9R)-2,3-dimethyl-8-hydroxy-7-(2-methylthioethyloxy)-9-phenyl-7,8,9,10-tetrahydroimidazo-[1,2-h][1,7]naphthyridine,
(7S,8R,9R)-2,3-dimethyl-8-hydroxy-7-(2-methylthioethyloxy)-9-phenyl-7,8,9,10-tetrahydroimidazo-[1,2-h][1,7]naphthyridine,
(7R,8R,9R)-2,3-dimethyl-8-hydroxy-7-(2-methylsulphinylethoxy)-9-phenyl-7,8,9,10-tetrahydroimidazo-[1,2-h][1,7]naphthyridine,
(7S,8R,9R)-2,3-dimethyl-8-hydroxy-7-(2-methylsulphinylethoxy)-9-phenyl-7,8,9,10-tetrahydroimidazo-[1,2-h][1,7]naphthyridine,
(7R,8R,9R)-2,3-dimethyl-8-hydroxy-7-(ethylthio)-9-phenyl-7,8,9,10-tetrahydroimidazo[1,2-h][1,7]naphthyridine,
(7S,8R,9R)-2,3-dimethyl-8-hydroxy-7-(ethylthio)-9-phenyl-7,8,9,10-tetrahydroimidazo[1,2-h][1,7]naphthyridine,
(7R,8R,9R)-2,3-dimethyl-8-hydroxy-7-(2,2,2-trifluoroethoxy)-9-phenyl-7,8,9,10-tetrahydroimidazo-[1,2-h][1,7]naphthyridine,
(7S,8R,9R)-2,3-dimethyl-8-hydroxy-7-(2,2,2-trifluoroethoxy)-9-phenyl-7,8,9,10-tetrahydroimidazo[1,2-h][1,7]naphthyridine,
(7S,8R,9R)-8-acetoxy-7-(2-methoxyethoxy)-2,3-dimethyl-9-phenyl-7,8,9,10-tetrahydroimidazo[1,2-h]-[1,7]naphthyridine,
(7R,8R,9R)-8-acetoxy-7-(2-methoxyethoxy)-2,3-dimethyl-9-phenyl-7,8,9,10-tetrahydroimidazo[1,2-h]-[1,7]naphthyridine,
(7R,8R,9R)-8-acetoxy-7-methoxy-2,3-dimethyl-9-phenyl-7,8,9,10-tetrahydroimidazo[1,2-h][1,7]naphthyridine,
(7R,8R,9R)-8-acetoxy-7-ethoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-9-phenyl-8-propionyloxy-7,8,9,10-tetrahydroimidazo-[1,2-h][1,7]naphthyridine,
(7R,8R,9R)-benzoyloxy-7-(2-methoxyethoxy)-2,3-dimethyl-9-phenyl-7,8,9,10-tetrahydroimidazo-[1,2-h][1,7]naphthyridine,
(7S,8R,9R)-8-benzoyloxy-7-(2-methoxyethoxy)-2,3-dimethyl-9-phenyl-7,8,9,10-tetrahydroimidazo-[1,2-h][1,7]naphthyridine,
(7R,8R,9R)-methoxycarbonyloxy-7-(2-methoxyethoxy)-2,3-dimethyl-9-phenyl-7,8,9,10-tetrahydroimidazo[1,2-h][1,7]naphthyridine,
(7S,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-7-methoxy-2,3-dimethyl-9-phenyl-7,8,9,10-tetrahydroimidazo[1,2-h][1,7]-naphthyridine,
(7S,8R,9R)-8-benzoyloxy-7-methoxy-2,3-dimethyl-9-phenyl-7,8,9,10-tetrahydroimidazo[1,2-h][1,7]-naphthyridine,
(7R,8R,9R)-8-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7S,8R,9R)-7-methoxy-2,3-dimethyl-8-(3-nitrobenzoyloxy)-9-phenyl-7,8,9,10-tetrahydroimidazo-[1,2-h][1,7]naphthyridine,
(7R,8R,9R)-7-methoxy-2,3-dimethyl-8-(3-nitrobenzoyloxy-9-phenyl-7,8,9,10-tetrahydroimidazo-[1,2-h][1,7]naphthyridine,
(7S,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-8-ethylaminocarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-2,3-dimethyl-7-(2-methoxyethoxy)-9-phenyl-7H-8,9-dihydropyrano[2,3-c]-imidazo[1,2-a]pyridine,
(7S,8R,9R-8-benzoyloxy-2,3-dimethyl-7-(2-methoxyethoxy)-9-phenyl-7H-8,9-dihydropyrano[2,3-c]-imidazo[1,2-a]pyridine,
(7R,8R,9R)-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-[4-(methoxycarbonyl)benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-2.3-dimethyl-7-methoxy-8-methoxyacetyloxy-9-phenyl-7.8.9.10-tetrahydroimidazo[1.2-h][1.7]naphthyridine,
(7R,8R,9R)-8-(N,N-diethylaminocarbonyloxy)-2.3-dimethyl-7-methoxy-9-phenyl-7.8.9.10-tetrahydroimidazo[1.2-h][1.7]naphthyridine,
(7S,8R,9R)-8-(N,N-diethylaminocarbonyloxy)-2.3-dimethyl-7-methoxy-9-phenyl-7.8.9.10-tetrahydroimidazo[1.2-h][1.7]naphthyridine,
(7R,8R,9R-7-methoxy-8-methoxycarbonyloxy-2.3-dimethyl-9-phenyl-7.8.9.10-tetrahydroimidazo-[1.2-h][1.7]naphthyridine,
(7S,8R,9R)-7-methoxy-8-methoxycarbonyloxy-2.3-dimethyl-9-phenyl-7.8.9.10-tetrahydroimidazo-[1.2-h][1.7]naphthyridine,
(7R,8R,9R)-2.3-dimethyl-8-formyloxy-7-methoxy-9-phenyl-7.8.9.10-tetrahydroimidazo[1.2-h][1.7]naphthyridine,
(7S,8R,9R)-2.3-dimethyl-8-formyloxy-7-methoxy-9-phenyl-7.8.9.10-tetrahydroimidazo[1.2-h][1.7]naphthyridine,
(7R,8R,9R)-8-benzoyloxy-2.3-dimethyl-7-methoxy-9-phenyl-7.8.9.10-tetrahydroimidazo[1.2-h][1.7]-naphthyridine,
(7R,8S,9R)-2,3,8-trimethyl-7,8-dihydroxy-9-phenyl-7,8,9,10-tetrahydroimidazo[1,2-h][1,7]naphthyridine,
(7S,8S,9R)-2,3-dimethyl-8-benzyl-7,8-dihydroxy-9-phenyl-7,8,9,10-tetrahydroimidazo[1,2-h][1,7]-naphthyridine,
(7R,8S,9R)-2,3,8-trimethyl-7,8-0,0-isopropylidene-9-phenyl-7,8,9,10-tetrahydroimidazo[1,2-h][1,7]-naphthyridine,
(7S,8S,9R)-2,3,8-trimethyl-7-(2-methoxyethoxy)-8-hydroxy-9-phenyl-7,8,9,10-tetrahydroimidazo-[1,2-h][1,7]naphthyridine,
(7S,8S,9R-2,3,8-trimethyl-7-methoxy-8-hydroxy-9-phenyl-7,8,9,1 0-tetrahydroimidazo[1,2-h][1,7]-naphthyridine,
(7R,8R,9R)-2,3,7-trimethyl-7,8-dihydroxy-9-phenyl-7,8,9,10-tetrahydroimidazo[1,2-h][1,7]naphthyridine,
(7R,8R,9R-2,3,7-trimethyl-7,8-[1,3]dioxolo-9-phenyl-7,8,9,10-tetrahydroimidazo[1,2-h][1,7]naphthyridine,
(8S,9R)-2,3-dimethyl-8-hydroxy-7-methylidene-9-phenyl-7,8,9,10-tetrahydroimidazo[1,2-h][1,7]-naphthyridine,
(7S,8R,9R)-2,3,7-trimethyl-7,8-dihydroxy-9-phenyl-7H-8,9-dihydropyrano[2,3-c]imidazo[1,2-a]pyridine,
(7R,8R,9R)-2,3,7-trimethyl-7,8-dihydroxy-9-phenyl-7H-8,9-dihydropyrano[2,3-c]imidazo[1,2-a]pyridine,
(7S,8R,9R)-2,3-dimethyl-7,8-dihydroxy-7,9-diphenyl-7H-8,9-dihydropyrano[2,3-c]imidazo[1,2-a]pyridine,
(7S,8R,9R)-2,3-dimethyl-7-(2′,2′-dimethylvinyl)-7,8-dihydroxy-9-phenyl-7H-8,9-dihydropyrano[2,3-c]-imidazo[1,2-a]pyridine,
(7R,8R,9R)-2,3-dimethyl-7,8-O-isopropylidene-9-phenyl-7-vinyl-7H-8,9-dihydropyrano[2,3-c]imidazo-[1,2-a]pyridine,
(7R,8R,9R)-2,3-dimethyl-8-hydroxy-7-(2-methoxyethoxy)-9-phenyl-7H-8,9-dihydropyrano[2,3-c]-imidazo[1,2-a]pyridine,
(7S,8R,9R)-2,3-dimethyl-8-hydroxy-7-(2-methoxyethoxy)-9-phenyl-7H-8,9-dihydropyrano[2,3-c]-imidazo[1,2-a]pyridine,
(7R,8R,9R)-2,3-dimethyl-8-hydroxy-7-ethoxy-9-phenyl-7H-8,9-dihydropyrano[2,3-c]imidazo[1,2-a]-pyridine,
(7S,8R,9R)-2,3-dimethyl-8-hydroxy-7-ethoxy-9-phenyl-7H-8,9-dihydropyrano[2,3-c]imidazo[1,2-a]-pyridine,
(7R,8R,9R)-2,3-dimethyl-8-hydroxy-7-(2-methoxypropoxy)-9-phenyl-7H-8,9-dihydropyrano[2,3-c]imidazo[1,2-a]pyridine,
(7S,8R,9R)-2,3-dimethyl-8-hydroxy-7-(2-methoxypropoxy)-9-phenyl-7H-8,9-dihydropyrano[2,3-c]imidazo[1,2-a]pyridine,
(7R,8R,9R)-2,3-dimethyl-8-hydroxy-7-(2-propoxy)-9-phenyl-7H-8,9-dihydropyrano[2,3-]imidazo-[1,2-a]pyridine,
(7S,8R,9R)-2,3-dimethyl-8-hydroxy-7-(2-propoxy)-9-phenyl-7H-8,9-dihydropyrano[2,3-c]imidazo-[1,2-a]pyridine,
(7R,8R,9R)-2,3-dimethyl-8-hydroxy-7-butoxy-9-phenyl-7H-8,9-dihydropyrano[2,3-c]imidazo[1,2-a]-pyridine,
(7S,8R,9R)-2,3-dimethyl-8-hydroxy-7-butoxy-9-phenyl-7H-8,9-dihydropyrano[2,3-c]imidazo[1,2-a]-pyridine,
(7S,8R,9R)-7,8-dihydroxy-6-methoxymethyl-2,3-dimethyl-9-phenyl-7,8,9,10-tetrahydroimidazo-[1,2-h][1,7]naphthyridine,
(7R,8R,9R)-7,8-dihydroxy-6-methoxymethyl-2,3-dimethyl-9-phenyl-7,8,9,10-tetrahydroimidazo[1,2-h]-[1,7]naphthyridine,
(7S,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-(2-methoxyethoxy)-methoxymethyl-2,3-dimethyl-9-phenyl-7,8,9,10-tetrahydroimidazo[1,2-h][1,7]naphthyridine,
(7S,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7R,8R,9R)-8-hydroxy-7-ethoxy-methoxymethyl-2,3-dimethyl-9-phenyl-7,8,9,10-tetrahydro-imidazo-[1,2-h][1,7]naphthyridine,
(7S,8R,9R)-8-hydroxy-7-ethoxy-6-methoxymethyl-2,3-dimethyl-9-phenyl-7,8,9,10-tetrahydro-imidazo-[1,2-h][1,7]naphthyridine,
7,8-dihydroxy-2,3-dimethyl-9-(3-thienyl)-7,8,9,10-tetrahydroimidazo[1,2-h][1,7]naphthyridine,
7-hydroxy-2,3-dimethyl-9-(3-thienyl)-7,8,9,10-tetrahydroimidazo[1,2-h][1,7]naphthyridine,
9-(3-furyl)-7-hydroxy-2,3-dimethyl-7,8,9,10-tetrahydroimidazo[1,2-h][1,7]naphthyridine,
(7R,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7S,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7R,8R,9R)-7,8-dihydroxy-2-methyl-9-phenyl-7,8,9,10-tetrahydroimidazo[1,2-h][1,7]naphthyridine,
(7S,8R,9R-8-hydroxy-2-methyl-7-(2-methoxyethoxy)-9-phenyl-7,8,9,10-tetrahydroimidazo[1,2-h]-[1,7]naphthyridine,
(7R,8R,9R)-8-hydroxy-2-methyl-7-(2-methoxyethoxy)-9-phenyl-7,8,9,10-tetrahydroimidazo[1,2-h]-[1,7]naphthyridine,
(7R,8R,9R)-3-bromo-8-hydroxy-7-(2-methoxyethoxy)-2-methyl-9-phenyl-7.8.9.10-tetrahydroimidazo-[1.2-h][1.7]naphthyridine,
(7R,8R,9R)-3-chloro-8-hydroxy-7-(2-methoxyethoxy)-2-methyl-9-phenyl-7.8.9.10-tetrahydroimidazo-[1.2-h][1.7]naphthyridine,
(7R,8R,9R)-3-bromo-7-hydroxy-8-(2-methoxyethoxy)-2-methyl-9-phenyl-7.8.9.10-tetrahydroimidazo-[1.2-h][1.7]naphthyridine,
(7R,8R,9R)-3-chloro-8-hydroxy-7-(2-methoxyethoxy)-2-methyl-9-phenyl-7H-8,9-dihydro-pyrano-[2,3-c]imidazo[1,2-a]pyridine,
(7R,8R,9R)-8-hydroxy-7-(2-methoxyethoxy)-2-methyl-9-phenyl-7H-8,9-dihydro-pyrano[2,3-cimidazo-[1,2-a]pyridine,
(7R,8R,9R)-7,8-dihydroxy-2-methyl-9-phenyl-7H-8,9-dihydropyrano[2,3-c]imidazo[1,2-a]pyridine,
(7S,8R,9R)-7,8-dihydroxy-2-methyl-9-phenyl-7.8.9.10-tetrahydroimidazo[1.2-h][1.7]naphthyridine,
(7R,8R,9R)-8-hydroxy-7-methoxy-2-methyl-9-phenyl-7.8.9.10-tetrahydroimidazo[1.2-h][1.7]naphthyridine,
(7S,8R,9R)-8-hydroxy-7-methoxy-2-methyl-9-phenyl-7.8.9.10-tetrahydroimidazo[1.2-h][1.7]naphthyridine,
(7R,8R,9R)-2,3-dimethyl-8-hydroxy-7-(2-hydroxyethoxy)-9-phenyl-7.8.9.10-tetrahydroimidazo[1.2-h][1.7]naphthyridine,
(7R,8R,9R)-3,9-diphenyl-8-hydroxy-7-(2-methoxyethoxy)-2-methyl-7-8.9.10-tetrahydroimidazo[1.2-h][1.7]naphthyridine,
(7R,8R,9R)-7,8-dihydroxy-2-methoxymethyl-3-methyl-9-phenyl-7,8,9,10-tetrahydroimidazo[1,2-h][1,7]naphthyridine,
(7S,8R,9R)-hydroxy-7-(2-methoxyethoxy)-2-methoxymethyl-3methyl-9-phenyl-7,8,9,10-tetrahydroimidazo[1,2-h][1,7]naphthyridine,
(7R,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7S,8R,9R)-7-ethoxy-8-hydroxy-2-methoxymethyl-3-methyl-9-phenyl-7,8,9,10-tetrahydroimidazo [1,2-h][1,7]naphthyridine,
(7R,8R,9R)-7-ethoxy-8-hydroxy-2-methoxymethyl-3-methyl-9-phenyl-7,8,9,10-tetrahydroimidazo-[1,2-h][1,7]naphthyridine,
(7R,8S,9R)-10-acetyl-8-hydroxy-2,3-dimethyl-7-(4-morpholino)-7,8,9,10-tetrahydroimidazo[1.2-h][1.7]naphthyridine,
(7R,8S,9R)-hydroxy-2,3-dimethyl-7-(4-morpholino)-7,8,9,10-tetrahydroimidazo[1.2-h][1.7]naphthyridine,
(7R,8S,9R)-10-acetyl-8-hydroxy-2,3-dimethyl-7-methylamino-7,8,9,10-tetrahydro-imidazo[1.2-h][1.7]naphthyridine,
(7R,8S,9R)-8-hydroxy-2,3-dimethyl-7-methylamino-7,8,9,10-tetrahydroimidazo[1.2-h]-[1.7]naphthyridine,
(7R,8S,9R)-10-acetyl-8-hydroxy-2,3-dimethyl-7-(1-pyrrolidino)-7,8,9,10-tetrahydroimidazo[1.2-h][1.7]naphthyridine,
(7R,8S,9R)-8-hydroxy-2,3-dimethyl-7-(1-pyrrolidino)-7,8,9,10-tetrahydroimidazo-[1.2-h][1.7]naphthyridine,
(7R,8S,9R)-10-acetyl-7-benzylamino-8-hydroxy-2,3-dimethyl-7,8,9,10-tetrahydro-imidazo[1.2-h][1.7]naphthyridine,
(7R,8S,9R)-7-benzylamino-8-hydroxy-2,3-dimethyl-7,8,9,10-tetrahydroimidazo[1.2-h][1.7]-naphthyridine,
(7R,8S,9R)-10-acetyl-8-hydroxy-7-(2-methoxyethylamino)-2,3-dimethyl-7,8,9,10-tetrahydroimidazo[1.2-h][1.7]naphthyridine,
(7R,8S,9R)-8-hydroxy-7-(2-methoxyethylamino)-2,3-dimethyl-7,8,9,10-tetrahydroimidazo[1.2-h][1.7]naphthyridine,
(7R,8S,9R)-10-acetyl-7-(dimethylamino)-8-hydroxy-2,3-dimethyl-7,8,9,10-tetrahydroimidazo[1.2-h][1.7]naphthyridine,
(7R,8S,9R)-8-hydroxy-7-(dimethylamino)-2,3-dimethyl-7,8,9,1 0-tetrahydroimidazo[1.2-h][1.7]naphthyridine,
(7S,8S,9R)-8-hydroxy-2,3,7-trimethyl-7,8,9,10-tetrahydroimidazo[1.2-h][1.7]naphthyridine,
(7S,8S,9R)-7-cyanomethyl-8-hydroxy-2,3-dimethyl-7,8,9,10-tetrahydroimidazo[1.2-h][1.7]naphthyridine,
(7S,8S,9R)-8-hydroxy-2,3-dimethyl-7-propyl-7,8,9,10-tetrahydroimidazo[1.2-h][1.7]naphthyridine,
(7R,8S,9R)-8-hydroxy-2,3-dimethyl-7-(3-methoxypropyl)-7,8,9,10-tetrahydroimidazo-[1.2-h][1.7]naphthyridine,
2,3-dimethyl-9-phenyl-7H-8,9-dihydro-pyrano-[2,3-c]-N-(diethyl)imidazo[1,2-a]pyridine-6-carboxamide,
ethyl 2,3-dimethyl-9-phenyl-7H-8,9-dihydro-pyrano[2,3-c]-imidazo[1,2-a]pyridine-6-carboxylate,
2,3-dimethyl-9-phenyl-7H-8,9-dihydro-pyrano[2,3-c]-imidazo[1,2-a]pyridine-6-(N,N-dimethyl)-carbamide,
(7R,8R,9R)-2,3-dimethyl-7-(2-methoxyethoxy)-9-phenyl-8-(5-nitrooxy-valeryloxy)-7,8,9,10-tetrahydroimidazo[1,2-h][1,7]naphthyridine,
(7R,8R,9R)-2,3-dimethyl-7-(2-methoxyethoxy)-9-phenyl-8-(4-nitrooxy-butyryloxy)-7,8,9,10-tetrahydroimidazo[1,2-h][1,7]naphthyridine,
(7R,8R,9R)-2,3-dimethyl-7-(2-methoxyethoxy)-9-phenyl-8-(5-nitro-oxy-valeryloxy)-7H-8,9-dihydropyrano[2,3-c]imdazo[1,2-a]pyridine,
(7R,8R,9R)-2,3-dimethyl-7-(2-methoxyethoxy)-9-phenyl-8-(6-nitro-oxy-2-oxa-capryloxy)-7,8,9,10-tetrahydro-midazo[1,2-h][1,7]naphthyridine and
(7R,8R,9R)-2,3-dimethyl-7-(2-methoxyethoxy)-9-phenyl-8-(4-nitro-oxymethyl-benzoyloxy)-7,8,9,10-tetrahydro-imidazo[1,2-h][1,7]naphthyridine,
   whereby BY-112 is thereof disclaimed,
   and the salts, solvates and solvates of the salts of these compounds.

Acid pump anatgonists according to a second detail of this invention (detail b), are, for example, described and/or claimed in the following patent applications and patents without being restricted to: EP 33094, EP 204285, EP 228006, EP 233760, EP 259174, EP 266326, EP 266890, EP 270091, EP 307078, EP 308917, EP 330485, U.S. Pat. No. 4,728,658, U.S. Pat. No. 5,362,743, WO 9212969, WO 9414795, WO 9418199, WO 9429274, WO 9510518, WO 9527714, WO 9603405, WO 9604251, WO 9605177, WO 9703074, WO 9703076, WO 9747603, WO 9837080, WO 9842707, WO 9843968, WO 9854188, WO 9909029, WO 9928322, WO 9950237, WO 9951584, WO 9955705, WO 9955706, WO 0001696, WO 0010999, WO 0011000, WO 0017200, WO 0026217, WO 0029403, WO 0063211, WO 0077003, WO 0158901, WO 0172754, WO 0172755, WO 0172756, WO 0172757, WO 0234749, WO 03014120, WO 03014123, WO 03016310 and WO 03018582, which are incorporated by reference into the specification of the present invention in their entirety for all purposes, and whereby particular emphasis is given in the present invention to those acid pump antagonists which are individualized and/or specifically disclosed and/or claimed in the abovementioned patent applications and patents.

As exemplary acid pump antagonists according to detail b the following compounds can be mentioned by means of their INNs or their research code acronyms: AG-2000 (EP 233760), AU-461 (WO 9909029), BY112 (WO 9842707), Soraprazan (WO 0017200), CP-113411 (U.S. Pat. No. 5,362,743), DBM-819 (WO 0001696), KR-60436 (WO 9909029), Pumaprazol (WO 9418199), SKF-96067 (EP 259174), SKF-96356 (EP 307078), SKF-97574 (EP 330485), T-330 (EP 270091), T-776 (EP 270091), WY-27198 (U.S. Pat. No. 4,728,658), YH-1885 (WO 9605177), YJA-20379-8 (WO 9703074) and YM-19020 (EP 266890).

As further exemplary acid pump antagonists according to detail b the following tricyclic imidazopyridine compounds listed in List B can likewise be mentioned.

List B consists of the following compounds:
(7S,8R,9R)-2,3-dimethyl-7,8-dihydroxy-9-phenyl-7,8,9,10-tetrahydroimidazo[1,2-h][1,7]naphthyridine,
(7R,8R,9R)-3-hydroxymethyl-7,8-dihydroxy-2-methyl-9-phenyl-7,8,9,10-tetrahydroimidazo[1,2-h][1,7-naphthyridine,
(7S,8R,9R-7,8-isopropylidenedioxy-2,3-dimethyl-9-phenyl-7,8,9,10-tetrahydroimidazo[1,2-h][1,7]naphthyridine,
7,8-dihydroxy-9-phenyl-2,3-dimethyl-7H-8,9-dihydropyrano[2,3-c]imidazo[1,2-a]pyridine,
(7R,8R,9R)-2,3-dimethyl-8-hydroxy-7-methoxy-9-phenyl-7,8,9,10-tetrahydroimidazo[1,2-h][1,7]naphthyridine,
(7S,8S,9S)-2,3-dimethyl-8-hydroxy-7-methoxy-9-phenyl-7,8,9,10-tetrahydroimidazo[1,2-h][1,7]naphthyridine,
(7S,8R,9R)-2,3-dimethyl-8-hydroxy-7-methoxy-9-phenyl-7,8,9,10-tetrahydroimidazo[1,2-h][1,7]naphthyridine,
(7R,8S,9S)-2,3-dimethyl-8-hydroxy-7-methoxy-9-phenyl-7,8,9,10-tetrahydroimidazo[1,2-h][1,7]naphthyridine,
(7R,8R,9R)-2,3-dimethyl-7-ethoxy-8-hydroxy-9-phenyl-7,8,9,10-tetrahydroimidazo[1,2-h][1,7]naphthyridine,
(7S,8R,9R)-2,3-dimethyl-7-ethoxy-8-hydroxy-9-phenyl-7,8,9,10-tetrahydroimidazo[1,2-h][1,7]naphthyridine,
(7R,8R,9R)-2,3-dimethyl-8-hydroxy-7-(2-methoxyethoxy)-9-phenyl-7,8,9,10-tetrahydroimidazo-[1,2-h][1,7]naphthyridine,
(7S,8S,9S)-2,3-dimethyl-8-hydroxy-7-(2-methoxyethoxy)-9-phenyl-7,8,9,10-tetrahydroimidazo-[1,2-h][1,7]naphthyridine,
(7S,8R,9R)-2,3-dimethyl-8-hydroxy-7-(2-methoxyethoxy)-9-phenyl-7,8,9,10-tetrahydroimidazo-[1,2-h][1,7]naphthyridine,
(7R,8S,9S)-2,3-dimethyl-8-hydroxy-7-(2-methoxyethoxy)-9-phenyl-7,8,9,10-tetrahydroimidazo-[1,2-h][1,7]naphthyridine,
(7S,8R,9R)-2,3-dimethyl-8-hydroxy-9-phenyl-7-(2-propoxy)-7,8,9,10-tetrahydroimidazo[1,2-h][1,7]-naphthyridine,
(7R,8R,9R)-2,3-dimethyl-7,8-dimethoxy-9-phenyl-7,8,9,10-tetrahydroimidazo[1,2-h][1,7]naphthyridine,
(7R,8R,9R)-2,3-dimethyl-8-hydroxy-7-(2-methylthioethyloxy)-9-phenyl-7,8,9,10-tetrahydroimidazo-[1,2-h][1,7]naphthyridine,
(7S,8R,9R)-2,3-dimethyl-8-hydroxy-7-(2-methylthioethyloxy)-9-phenyl-7,8,9,10-tetrahydroimidazo-[1,2-h][1,7]naphthyridine,
(7R,8R,9R)-2,3-dimethyl-8-hydroxy-7-(2-methylsulphinylethoxy)-9-phenyl-7,8,9,10-tetrahydroimidazo-[1,2-h][1,7]naphthyridine,
(7S,8R,9R)-2,3-dimethyl-8-hydroxy-7-(2-methylsulphinylethoxy 9-phenyl-7,8,9,10-tetrahydroimidazo-[1,2-h][1,7]naphthyridine,
(7R,8R,9R)-2,3-dimethyl-8-hydroxy-7-(ethylthio)-9-phenyl-7,8,9,10-tetrahydroimidazo[1,2-h][1,7]naphthyridine,
(7S,8R,9R)-2,3-dimethyl-8-hydroxy-7-(ethylthio)-9-phenyl-7,8,9,10-tetrahydroimidazo[1,2-h][1,7]naphthyridine,
(7R,8R,9R)-2,3-dimethyl-8-hydroxy-7-(2,2,2-trifluoroethoxy)-9-phenyl-7,8,9,10-tetrahydroimidazo-[1,2-h][1,7]naphthyridine,
(7S,8R,9R)-2,3-dimethyl-8-hydroxy-7-(2,2,2-trifluoroethoxy)-9-phenyl-7,8,9,10-tetrahydroimidazo[1,2-h][1,7]naphthyridine,
(7S,8R,9R)-8-acetoxy-7-(2-methoxyethoxy)-2,3-dimethyl-9-phenyl-7,8,9,10-tetrahydroimidazo[1,2-h]-[1,7]naphthyridine,
(7R,8R,9R)-8-acetoxy-7-(2-methoxyethoxy)-2,3-dimethyl-9-phenyl-7,8,9,10-tetrahydroimidazo[1,2-h]-[1,7]naphthyridine,
(7R,8R,9R)-8-acetoxy-7-methoxy-2,3-dimethyl-9-phenyl-7,8,9,10-tetrahydroimidazo[1,2-h][1,7]naphthyridine,
(7R,8R,9R)-8-acetoxy-7-ethoxy-2,3-dimethyl-9-phenyl-7,8,9,10-tetrahydroimidazo[1,2-h][1,7]naphthyridine,
(7R,8R,9R-7-(2-methoxyethoxy)-2,3-dimethyl-9-phenyl-8-propionyloxy-7,8,9,10-tetrahydroimidazo-[1,2-h][1,7]naphthyridine,
(7R,8R,9R)-8-benzoyloxy-7-(2-methoxyethoxy)-2,3-dimethyl-9-phenyl-7,8,9,10-tetrahydroimidazo-[1,2-h][1,7]naphthyridine,
(7S,8R,9R)-8-benzoyloxy-7-(2-methoxyethoxy)-2,3-dimethyl-9-phenyl-7,8,9,10-tetrahydroimidazo-[1,2-h][1,7]naphthyridine,
(7R,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7S,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-7-methoxy-2,3-dimethyl-9-phenyl-7,8,9,10-tetrahydroimidazo[1,2-h][1,7]-naphthyridine,
(7S,8R,9R)-8-benzoyloxy-7-methoxy-2,3-dimethyl-9-phenyl-7,8,9,10-tetrahydroimidazo[1,2-h][1,7]-naphthyridine,
(7R,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7S,8R,9R)-7-methoxy-2,3-dimethyl-8-(3-nitrobenzoyloxy)-9-phenyl-7,8,9,10-tetrahydroimidazo-[1,2-h][1,7]naphthyridine,
(7R,8R,9R)-7-methoxy-2,3-dimethyl-8-(3-nitrobenzoyloxy)-9-phenyl-7,8,9,10-tetrahydroimidazo-[1,2-h][1,7]naphthyridine,
(7S,8R,9R)-7-(2-methoxyethoxy)-2,3-dimethyl-8-4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-7-(2-methoxyethoxy)-(N,N-diethylaminocarbonyloxy)-2,3-dimethyl-9-phenyl-7,8,9,10-tetrahydroimidazo[1,2-h][1,7]naphthyridine,
(7R,8R,9R)-8-ethylaminocarbonyloxy-7-(2-methoxyethoxy)-2,3-dimethyl-9-phenyl-7,8,9,1-tetrahydroimidazo[1,2-h][1,7]naphthyridine,
(7R,8R,9R)-8-benzoyloxy-2,3-dimethyl-7-(2-methoxyethoxy)-9-phenyl-7H-8,9-dihydropyrano[2,3-c]-imidazo[1,2-a]pyridine,
(7S,8R,9R)-8-benzoyloxy-2,3-dimethyl-7-(2-methoxyethoxy)-9-phenyl-7H-8,9-dihydropyrano[2,3-c]-imidazo[1,2-a]pyridine,
(7R,8R,9R)-8-[(methoxycarbonylbenzoyloxy]-2,3-dimethyl-7-(2-methoxyethoxy)-9-phenyl-7H-8,9-dihydropyrano[2,3-c]imidazo[1,2-a]pyridine,
(7S,8R,9R)-8-(4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-2.3-dimethyl-7-methoxy-8-methoxyacetyloxy-9-phenyl-7.8.9.10-tetrahydroimidazo[1.2-h]-[1.7]naphthyridine,
(7R,8R,9R)-8-(N,N-diethylaminocarbonyloxy)-2.3-dimethyl-7-methoxy-9-phenyl-7.8.9.10-tetrahydroimidazo[1.2-h][1.7]naphthyridine,
(7S,8R,9R)-8-(N,N-diethylaminocarbonyloxy)-2.3-dimethyl-7-methoxy-9-phenyl-7.8.9.10-tetrahydroimidazo[1.2-h][1.7]naphthyridine,
(7R,8R,9R)-7-methoxy-8-methoxycarbonyloxy-2.3-dimethyl-9-phenyl-7.8.9.10-tetrahydroimidazo-[1.2-h][1.7]naphthyridine,
(7S,8R,9R)-7-methoxy-8-methoxycarbonyloxy-2.3-dimethyl-9-phenyl-7.8.9.10-tetrahydroimidazo-[1.2-h][1.7]naphthyridine,
(7R,8R,9R)-2.3-dimethyl-8-formyloxy-7-methoxy-9-phenyl-7.8.9.10-tetrahydroimidazo[1.2-h][1.7]naphthyridine,
(7S,8R,9R)-2.3-dimethyl-8-formyloxy-7-methoxy-9-phenyl-7.8.9.10-tetrahydroimidazo[1.2-h][1.7]naphthyridine,
(7R,8R,9R)-8-benzoyloxy-2.3-dimethyl-7-methoxy-9-phenyl-7.8.9.10-tetrahydroimidazo[1.2-h][1.7]-naphthyridine,
(7R,8S,9R)-2,3,8-trimethyl-7,8-dihydroxy-9-phenyl-7,8,9,10-tetrahydroimidazo[1,2-h][1.7]naphthyridine,
(7S,8S,9R)-2,3-dimethyl-4-benzyl-7,8-dihydroxy-9-phenyl-7,8,9,10-tetrahydroimidazo[1,2-h]-[1,7]naphthyridine,
(7R,8S,9R)-2,3,8-trimethyl-7,8-0,0-isopropylidene-9-phenyl-7,8,9,10-tetrahydroimidazo[1,2-h][1,7]-naphthyridine,
(7S,8S,9R-2,3,8-trimethyl-7-(2-methoxyethoxy)-8-hydroxy-9-phenyl-7,8,9,10-tetrahydroimidazo-[1,2-h][1,7]naphthyridine,
(7S,8S,9R)-2,3,8-trimethyl-7-methoxy-8-hydroxy-9-phenyl-7,8,9,10-tetrahydroimidazo[1,2-h][1,7]-naphthyridine,
(7R,8R,9R)-2,3,7-trimethyl-7,8-dihydroxy-9-phenyl-7,8,9,10-tetrahydroimidazo[1,2-h][1,7]naphthyridine,
(7R,8R,9R)-2,3,7-trimethyl-7,8-[1,3]dioxolo-9-phenyl-7,8,9,10-tetrahydroimidazo[1,2-h][1,7]naphthyridine,
(8S,9R)-2,3-dimethyl-8-hydroxy-7-methylidene-9-phenyl-7,8,9,10-tetrahydroimidazo[1,2-h][1,7]-naphthyridine,
(7S,8R,9R)-2,3,7-trimethyl-7,8-dihydroxy-9-phenyl-7H-8,9-dihydropyrano[2,3-c]imidazo[11,2-a]pyridine,
(7R,8R,9R)-2,3,7-trimethyl-7,8-dihydroxy-9-phenyl-7H-8,9-dihydropyrano[2,3-c]imidazo[1,2-a]pyridine,
(7S,8R,9R)-2,3-dimethyl-7,8-dihydroxy-7,9-diphenyl-7H-8,9-dihydropyrano[2,3-c]imidazo[1,2-a]pyridine,
(7S,8R,9R)-2,3-dimethyl-7-(2′,2′-dimethylvinyl)-7,8-dihydroxy-9-phenyl-7H-8,9-dihydropyrano[2,3-c]-imidazo[1,2-a]pyridine,
(7R,8R,9R)-2,3-dimethyl-7,8-0,0-isopropylidene-9-phenyl-7-vinyl-7H-8,9-dihydropyrano[2,3-c]imidazo-[1,2-a]pyridine,
(7R,8R,9R)-2,3-dimethyl-8-hydroxy-7-(2-methoxyethoxy)-9-phenyl-7H-8,9-dihydropyrano[2,3-c]-imidazo[1,2-a]pyridine,
(7S,8R,9R)-2,3-dimethyl-8-hydroxy-7-(2-methoxyethoxy)-9-phenyl-7H-8,9-dihydropyrano[2,3-c]imidazo[1,2-a]pyridine,
(7R,8R,9R)-2,3-dimethyl-8-hydroxy-7-ethoxy-9-phenyl-7H-8,9-dihydropyrano[2,3-c]imidazo[1,2-a]-pyridine,
(7S,8R,9R)-2,3-dimethylhydroxy-7-ethoxy-9-phenyl-7H-8,9-dihydropyrano[2,3-c]imidazo[1,2-a]-pyridine,
(7R,8R,9R)-2,3-dimethyl-8-hydroxy-7-(2-methoxypropoxy)-9-phenyl-7H-8,9-dihydropyrano[2,3-c]imidazo[1,2-a]pyridine,
(7S,8R,9R)-2,3-dimethyl-8-hydroxy-7-(2-methoxypropoxy)-9-phenyl-7H-8,9-dihydropyrano[2,3-c]imidazo[1,2-a]pyridine,
(7R,8R,9R)-2,3-dimethyl-8-hydroxy-7-(2-propoxy)-9-phenyl-7H-8,9-dihydropyrano[2,3-cimidazo[1,2-a]pyridine,
(7S,8R,9R)-2,3-dimethyl-8-hydroxy-7-(2-propoxy)-9-phenyl-7H-8,9-dihydropyrano[2,3-c]imidazo[1,2-a]pyridine,
(7R,8R,9R)-2,3-dimethyl-8-hydroxy-7-butoxy-9-phenyl-7H-8,9-dihydropyrano[2,3-c]imidazo[1,2-a]-pyridine,
(7S,8R,9R)-2,3-dimethyl-8-hydroxy-7-butoxy-9-phenyl-7H-8,9-dihydropyrano[2,3-c]imidazo[1,2-a]-pyridine,
(7S,8R,9R)-7,8-dihydroxy-6-methoxymethyl-2,3-dimethyl-9-phenyl-7,8,9,10-tetrahydroimidazo[1,2-h]-[1,7]naphthyridine,
(7R,8R,9R)-7,8-dihydroxy-6-methoxymethyl-2,3-dimethyl-9-phenyl-7,8,9,10-tetrahydroimidazo[1,2-h]-[1,7]naphthyridine,
(7S,8R,9R-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7S,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7R,8R,9R)-8-hydroxy-7-ethoxy-6-methoxymethyl-2,3-dimethyl-9-phenyl-7,8,9,10-tetrahydro-imidazo-[1,2-h][1,7]naphthyridine,
(7S,8R,9R)-8-hydroxy-7-ethoxy-6-methoxymethyl-2,3-dimethyl-9-phenyl-7,8,9,10-tetrahydro-imidazo-[1,2-h][1,7]naphthyridine,
7,8-dihydroxy-2,3-dimethyl-9-(3-thienyl)-7,8,9,10-tetrahydroimidazo[1,2-h][1,7]naphthyridine,
7-hydroxy-2,3-dimethyl-9-(3-thienyl)-7,8,9,10-tetrahydroimidazo[1,2-h][1,7]naphthyridine,
9-(3-furyl)-7-hydroxy-2,3-dimethyl-7,8,9,10-tetrahydroimidazo[1,2-h][1,7]naphthyridine,
(7R,8R,9R)-8-hydroxy-7-[2-(2-methoxyethoxy)ethoxy]-2,3-dimethyl-9-phenyl-7,8,9,10-tetrahydroimidazo1,2-h][1,7]naphthyridine,
(7S,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7R,8R,9R)-7,8-dihydroxy-2-methyl-9-phenyl-7,8,9,10-tetrahydroimidazo[1,2-h][1,7]naphthyridine,
(7S,8R,9R)-8-hydroxy-2-methyl-7-(2-methoxyethoxy)-9-phenyl-7,8,9,10-tetrahydroimidazo[1,2-h]-[1,7]naphthyridine,
(7R,8R,9R)-8-hydroxy-2-methyl-7-(2-methoxyethoxy)-9-phenyl-7,8,9,10-tetrahydroimidazo[1,2-h]-[1,7]naphthyridine,
(7R,8R,9R)-3-bromo-8-hydroxy-7-(2-methoxyethoxy)-2-methyl-9-phenyl-7.8.9.10-tetrahydroimidazo-[1.2-h][1.7]naphthyridine,
(7R,8R,9R)-3-chloro-8-hydroxy-7-(2-methoxyethoxy)-2-methyl-9-phenyl-7.8.9.10-tetrahydroimidazo-[1.2-h][1.7]naphthyridine,
(7R,8R,9R)-3-bromo-7-hydroxy-8-(2-methoxyethoxy)-2-methyl-9-phenyl-7.8.9.10-tetrahydroimidazo-[1.2-h][1.7]naphthyridine,
(7R,8R,9R)-3-chloro-8-hydroxy-7-(2-methoxyethoxy)-2-methyl-9-phenyl-7H-8,9-dihydro-pyrano[2,3-c]-imidazo[1,2-a]pyridine,
(7R,8R,9R)-8-hydroxy-7-(2-methoxyethoxy)-2-methyl-9-phenyl-7H-8,9-dihydro-pyrano[2.3-c]imidazo-[1,2-a]pyridine,
(7R,8R,9R)-7,8-dihydroxy-2-methyl-9-phenyl-7H-8,9-dihydropyrano[2,3-c]imidazo[1,2-a]pyridine,
(7S,8R,9R)-7,8-dihydroxy-2-methyl-9-phenyl-7.8.9.10-tetrahydroimidazo[1.2-h][1.7]naphthyridine,
(7R,8R,9R)-8-hydroxy-7-methoxy-2-methyl-9-phenyl-7.8.9.10-tetrahydroimidazo[1.2-h][1.7]naphthyridine,
(7S,8R,9R)-8-hydroxy-7-methoxy-2-methyl-9-phenyl-7.8.9.10-tetrahydroimidazo[1.2-h][1.7]naphthyridine,
(7R,8R,9R)-3-hydroxymethyl-8-hydroxy-7-(2-methoxyethoxy)-2-methyl-9-phenyl-7.8.9.10-tetrahydroimidazo[1.2-h][1.7]naphthyridine,
(7R,8R,9R)-3-hydroxymethyl-8-hydroxy-7-(2-hydroxyethoxy)-2-methyl-9-phenyl-7.8.9.10-tetrahydroimidazo[1.2-h][1.7]naphthyridine,
(7R,8R,9R)-2,3-dimethyl-8-hydroxy-7-(2-hydroxyethoxy)-9-phenyl-7.8.9.10-tetrahydroimidazo[1.2-h]-[1.7]naphthyridine,
(7R,8R,9R)-3,9-diphenyl-8-hydroxy-7-(2-methoxyethoxy)-2-methyl-7.8.9.10-tetrahydroimidazo[1.2-h]-[1.7]naphthyridine,
(7R,8R,9R)-7,8-dihydroxy-2-methoxymethyl-3-methyl-9-phenyl-7,8,9,10-tetrahydroimidazo[1,2-h]-[1,7]naphthyridine,
(7S,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7R,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7S,8R,9R)-7-ethoxy-8-hydroxy-2-methoxymethyl-3-methyl-9-phenyl-7,8,9,10-tetrahydroimidazo[1,2-h][1,7]naphthyridine,
(7R,8R,9R)-7-ethoxy-8-hydroxy-2-methoxymethyl-3-methyl-9-phenyl-7,8,9,10-tetrahydroimidazo[1,2-h][1,7]naphthyridine,
(7R,8S,9R)-10-acetyl-8-hydroxy-2,3-dimethyl-7-(4-morpholino)-7,8,9,10-tetrahydroimidazo[1.2-h][1.7]naphthyridine,
(7R,8S,9R)-8-hydroxy-2,3-dimethyl-7-(4-morpholino)-7,8,9,10-tetrahydroimidazo[1.2-h]-[1.7]naphthyridine,
(7R,8S,9R)-10-acetyl-8-hydroxy-2,3-dimethyl-7-methylamino-7,8,9,10-tetrahydro-imidazo[1.2-h][1.7]naphthyridine,
(7R,8S,9R)-8-hydroxy-2,3-dimethyl-7-methylamino-7,8,9,10-tetrahydroimidazo[1.2-h]-[1.7]naphthyridine,
(7R,8S,9R)-10-acetyl-8-hydroxy-2,3-dimethyl-7-(1-pyrrolidino)-7,8,9,10-tetrahydroimidazo[1.2-h][1.7]naphthyridine,
(7R,8S,9R)-8-hydroxy-2,3-dimethyl-7-(1-pyrrolidino)-7,8,9,10-tetrahydroimidazo[1.2-h]-[1.7]naphthyridine,
(7R,8S,9R)-10-acetyl-7-benzylamino-8-hydroxy-2,3-dimethyl-7,8,9,10-tetrahydro-imidazo[1.2-h]-[1.7]naphthyridine,
(7R,8S,9R-7-benzylamino-8-hydroxy-2,3-dimethyl-7,8,9,10-tetrahydroimidazo[1.2-h][1.7]-naphthyridine,
(7R,8S,9R)-10-acetyl-8-hydroxy-7-(2-methoxyethylamino)-2,3-dimethyl-7,8,9,10-tetrahydroimidazo[1.2-h][1.7]naphthyridine,
(7R,8S,9R)-8-hydroxy-7-(2-methoxyethylamino)-2,3-dimethyl-7,8,9,10-tetrahydroimidazo[1.2-h][1.7]naphthyridine,
(7R,8S,9R)-10-acetyl-7-dimethylamino)-8-hydroxy-2,3-dimethyl-7,8,9,10-tetrahydro-imidazo[1.2-h][1.7]naphthyridine,
(7R,8S,9R)-8-hydroxy-7-(dimethylamino)-2,3-dimethyl-7,8,9,10-tetrahydroimidazo[1.2-h]-[1.7]naphthyridine,
(7S,8S,9R)-8-hydroxy-2,3,7-trimethyl-7,8,9,10-tetrahydroimidazo[1.2-h][1.7]naphthyridine,
(7S,8S,9R)-7-Cyanomethyl-8-hydroxy-2,3-dimethyl-7,8,9,10-tetrahydroimidazo[1,2h]-[1.7]naphthyridine,
(7S,8S,9R)-8-hydroxy-2,3-dimethyl-7-propyl-7,8,9,10-tetrahydroimidazo[1.2-h][1.7]naphthyridine,
(7R,8S,9R)-8-Hydroxy-2,3-dimethyl-7-(3-methoxypropyl)-7,8,9,10-tetrahydroimidazo-[1.2-h][1.7]naphthyridine,
2,3-dimethyl-phenyl-7H-8,9-dihydro-pyrano-[2,3-c]-N-(diethyl)imidazo[1,2-a]pyridine-6-carboxamide,
ethyl 2,3-dimethyl-9-phenyl-7H-8,9-dihydro-pyrano[2,3-c]-imidazo[1,2-a]pyridine-6-carboxylate and
2,3-dimethyl-9-phenyl-7H-8,9-dihydro-pyrano[2,3-c]-imidazo[1,2-a]pyridine-6-(N,N-dimethyl)-carbamide.

Acid pump anatgonists according to a third detail of this invention (detail c), are, for example, those bicyclic imidazopyridines which are claimed and/or described specifically or generically in the patent applications WO 9955706, WO 03018582 and/or, particularly, WO04/000855 and/or WO04/000856, which are all incorporated by reference into the specification of the present invention in their entirety for all purposes, and whereby particular emphasis is given in detail c of the present invention to those acid pump antagonists which are individualized (e.g. mentioned expressis verbis) and/or specifically disclosed and/or claimed in the abovementioned patent applications.

As exemplary acid pump antagonists according to detail c can be mentioned any imidazopyridine compound selected from the group (group x) consisting of
2,3-dimethyl-8-(2-ethyl-6-methylbenzylamino)-N-propyl-imidazo[1,2-a]pyridine-6-carboxamide,
8-(2-ethyl-6-methylbenzylamino)-3-hydroxymethyl-2-methylimidazol-1,2-a)pyridine-6-carboxamide,
2,3-dimethyl-(2,6-dimethylbenzylamino)-N-hydroxyethyl-imidazo[1,2-a]pyridine-6-carboxamide,
2,3-dimethyl-8-(2-ethyl-6-methylbenzylamino)-imidazo[1,2-a]pyridine-6-carboxamide,
8-(2-ethyl-6-methylbenzylamino)-N,2,3-trimethylimidazo[1,2-a]pyridine-6-carboxamide,
8-(2-ethyl-6-methylbenzylamino)-N,N,2,3-tetramethylimidazo[1,2-a]pyridine-6-carboxamide,
2,3-dimethyl-8-(2,6-dimethylbenzyl-amino)-imidazo[1,2-a]pyridine-6-carboxamide,
N-[2-(dimethylamine)-2-oxoethyl]-8-(2-ethyl-6-methylbenzylamino)-N,2,3-trimethylimidazo[1,2-a]pyridine-6-carboxamide,
2,3-dimethyl-8-(2-ethyl-4-fluoro-6-methylbenzylamino)-imidazo[1,2-a]-pyridine carboxamide mesylate,
2,3-dimethyl-8-(2-methylbenzylamino)-imidazo[1,2-a]pyridine-6-carboxamide,
2,3-dimethyl-8-(2,6-dimethylfluoro-benzylamino)-imidazo[1,2-a]pyridine-6-carboxamide mesylate,
2,3-dimethyl-8-(2-methyl-6-isopropylbenzylamino)imidazo[1,2-a]pyridine-6-carboxamide mesylate,
2,3-dimethyl-8-(2,6-diethyl-benzylamino)imidazo[1,2-a]pyridine-6-carboxamide,
2,3-dimethyl-8-(2-ethylbenzylamino)-imidazo[1,2-a]pyridine-6-carboxamide,
2,3 dimethyl-8-(2-ethyl-6-methyl-benzylamino)-N-hydroxyethyl-imidazo[1,2-a]pyridine-6-carboxamide,
N-(2,3-dihydroxypropyl)-2,3-dimethyl-4-(2-ethyl-6-methylbenzylamino)-[1,2-a]pyridine-6-carboxamide,
2,3 dimethyl-8-(2-ethyl-6-methyl-benzylamino)-N-(2-methoxyethyl)-imidazo[1,2-a]pyridine-6-carboxamide,
2-methyl-8-(2-ethyl-6-methylbenzylamino)-imidazo[1,2-a]pyridine-6-carboxamide,
2,3-dimethyl-8-(2-bromo-6-methylbenzylamino)-imidazo[1,2-a]pyridine-6-carboxamide,
2,3-dimethyl-8-(2-(2-hydroxyethyl)-6-methylbenzylamino)-imidazo[1,2-a]pyridine-6-carboxamide,
8-(2-ethyl-6-methylbenzylamino)-N,N-bis(2-hydroxyethyl)-2,3-dimethylimidazo[1,2-a]pyridine-6-carboxamide,
8-(2-ethyl-6-methylbenzylamino)-N-(2-hydroxyethyl)-2,3-trimethylimidazo[1,2-a]pyridine-6-carboxamide,
   and 2,3-dimethyl-8-(2-ethyl-6-methylbenzyloxy)-imidazo[1,2-a]pyridine-6-carboxamide,
   or a pharmaceutically acceptable salt thereof.

As further exemplary acid pump antagonists according to detail c can be also mentioned any imidazopyridine compound selected from the group (group y) consisting of
8-(2-ethyl-6-methylbenzylamino)-3-hydroxymethyl-2-methylimidazo[1,2-a]-pyridine-6-carboxamide,
2,3-dimethyl-8-(2,6-dimethylbenzylamino)-N-hydroxyethyl-imidazo[1,2-a]pyridine-6-carboxamide,
2,3-dimethyl-8-(2-ethyl-6-methylbenzylamino)-imidazo[1,2-a]pyridine-6-carboxamide,
8-(2-ethyl-6-methylbenzylamino)-N,2,3-trimethylimidazo[1,2-a]pyridine-6-carboxamide,
2,3-dimethyl-8-(2,6-dimethylbenzylamino)-imidazo[1,2-a]-pyridine-6-carboxamide,
2,3-dimethyl-8-(2-ethyl-4-fluoro-6-methylbenzylamino)-imidazo[1,2-a]pyridine-6-carboxamide,
2,3-dimethyl-8-(2,6-dimethyl fluoro-benzylamino)-imidazo[1,2-a]pyridine-6-carboxamide,
2,3-dimethyl-8-(2,6-dimethylbenzylamino)-imidazo[1,2-a]pyridine-6-carboxamide,
2,3 dimethyl-8-(2-ethyl-6-methylbenzylamino)-N-hydroxyethyl-imidazo[1,2-a]pyridine-6-carboxamide,
   and 2,3 dimethyl-8-(2-ethyl-6-methylbenzylamino)-N-(2-methoxyethyl)-imidazo[1,2-a]pyridine-6-carboxamide,
   or a pharmaceutically acceptable salt thereof.

In the context thereof, to be mentioned in an independent embodimental aspect is AZD-0865.

Preferred acid pump antagonists according to detail a of this invention are those compounds which are mentioned expressis verbis in the abovementioned List A,

and the salts, solvates and solvates of the salts of these compounds.

A suitable tricyclic Imidazo[1,2-a]pyridine compound according to detail a and/or detail b of this invention in particular to be emphasized is (7R,8R,9R)-8-hydroxy-7-(2-methoxyethoxy)-2,3-dimethyl-9-phenyl-7,8,9,10-tetrahydro-imidazo[1,2-h][1,7]naphthyridine or a salt, solvate or solvate of a salt of this compound.

In particular preferred acid pump antagonists according to detail a of this invention are compounds selected from the group consisting of those tricyclic Imidazo[1,2-a]pyridine compounds mentioned expressis verbis in the following List C, and the salts, solvates and solvates of the salts of these compounds.

List C consists of the following specific compounds:
1. (7R,8R,9R)-8-hydroxy-7-(2-methoxyethoxy)-2,3-dimethyl-9-phenyl-7,8,9,10-tetrahydroimidazo[1,2-h][1,7]naphthyridine,
2. (7R,8R,9R)-2,3-dimethyl-8-hydroxy-7-methoxy-9-phenyl-7,8,9,10-tetrahydroimidazo[1,2-h][1,7]naphthyridine,
3. (7S,8R,9R)-2,3-dimethyl-8-hydroxy-7-methoxy-9-phenyl-7,8,9,10-tetrahydroimidazo[1,2-h][1,7]naphthyridine,
4. (7R,8R,9R)-2,3-dimethyl-7-ethoxy-8-hydroxy-9-phenyl-7,8,9,10-tetrahydroimidazo[1,2-h][1,7]naphthyridine,
5. (7S,8R,9R)-2,3-dimethyl-7-ethoxy-8-hydroxy-9-phenyl-7,8,9,10-tetrahydroimidazo[1,2-h][1,7]naphthyridine,
6. (7R,8R,9R)-2,3-dimethyl-8-hydroxy-7-(2-methoxyethoxy)-9-phenyl-7,8,9,10-tetrahydroimidazo[1,2-h][1,7]naphthyridine,
7. (7R,8R,9R)-8-acetoxy-7-(2-methoxyethoxy)-2,3-dimethyl-9-phenyl-7,8,9,10-tetrahydroimidazo[1,2-h][1,7]naphthyridine,
8. (7R,8R,9R)-8-benzoyloxy-7-(2-methoxyethoxy)-2,3-dimethyl-9-phenyl-7,8,9,10-tetrahydroimidazo[1,2-h][1,7]naphthyridine,
9. (7R,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
10. (7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
11. (7R,8S,9R)-2,3,8-trimethyl-7,8-dihydroxy-9-phenyl-7,8,9,10-tetrahydroimidazo[1,2-h][1,7]naphthyridine,
12. (7R,8R,9R)-2,3,7-trimethyl-7,8-dihydroxy-9-phenyl-7,8,9,10-tetrahydroimidazo[1,2-h][1,7]naphthyridine,
13. (7R,8R,9R)-2,3-dimethyl-hydroxy-7-(2-methoxyethoxy)-9-phenyl-7H-8,9-dihydropyrano[2,3-c]imidazo[1,2-a]pyridine,
14. (7R,8R,9R)-2,3-dimethyl-8-hydroxy-7-ethoxy-9-phenyl-7H-8,9-dihydropyrano[2,3-c]imidazo-[1,2-a]pyridine,
15. (7R,8R,9R)-8-hydroxy-2-methyl-7-(2-methoxyethoxy)-9-phenyl-7,8,9,10-tetrahydroimidazo[1,2-h][1,7]naphthyridine,
16. (7R,8R,9R)-3-chloro-8-hydroxy-7-(2-methoxyethoxy)-2-methyl-9-phenyl-7.8.9.10-tetrahydroimidazo[1.2-h][1.7]naphthyridine, and
17. (7R,8R,9R)-3-chloro-8-hydroxy-7-(2-methoxyethoxy)-2-methyl-9-phenyl-7H-8,9-dihydropyrano[2,3-c]imidazo[1,2-a]pyridine,
   and the salts, solvates and solvates of the salts thereof.

According to the present invention it is to be stated that any or all of the tricyclic imidazo[1,2-a]pyridine compounds mentioned expressis verbis in List C, as well as the salts, solvates and solvates of the salts thereof, are useful within this invention and are suitable to be used in the combination therapy, combinations or compositions according to this invention together with compounds, which modify gastrointestinal motility, as described herein.

In more detail, it is to be stated within the scope of this invention, that each single individual tricyclic imidazo[1,2-a]pyridine compound mentioned expressis verbis in List C as compound 1 to 17 as well as a salt, solvate or solvate of a salt thereof can be individually paired, each in independent specific special embodiments according to the present invention, with any compound or class of compounds, which modify gastrointestinal motility, as defined herein in combinations or compositions according to this invention, or for use in combination therapies as described herein.

The compounds mentioned in List A, B, or C as well as the salts, solvates and solvates of the salts thereof and their preparation are described in greater details in the applications mentioned in details a or b, respectively.

Particularly worthy to be mentioned of the acid pump antagonists according to detail b are the compounds AU-461, Soraprazan, DBM-819, KR-60436, T-330, YH-1885 and YJA-20379-8, especially Soraprazan and YH-1885.

As exemplary preferred acid pump antagonists according to detail a and/or detail b the compounds
(7R,8R,9R)-2.3-dimethyl-8-hydroxy-7-(2-methoxyethoxy)-9-phenyl-7.8.9.10-tetrahydro-imidazo-[1.2-h][1.7]naphthyridin,
(7R,8R,9R)-2,3-dimethyl-8-hydroxy-7-(2-methoxyethoxy)-9-phenyl-7H-8,9-dihydro-pyrano[2,3-c]-imidazo[1,2-a]pyridine and
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h)[1,7]naphthyridine are to be mentioned.

The acid pump antagonists are available as such or in the form of their salts. Suitable salts in the scope of this invention are especially all acid addition salts. Particular mention may be made of the pharmacologically tolerable salts of the inorganic or organic acids customarily used in pharmacy. Those suitable are water-insoluble and in particular water-soluble acid addition salts with acids such as, for example, hydrochloric acid, hydrobromic acid, phosphoric acid, nitric acid, sulfuric acid, acetic acid, citric acid, D-gluconic acid, benzoic acid, 2-(4-hydroxybenzoyl)benzoic acid, butyric acid, sulfosalicylic acid, maleic acid, lauric acid, malic acid, fumaric acid, succinic acid, oxalic acid, tartaric acid, embonic acid, stearic acid, toluenesulfonic acid, methanesulfonic acid or 3-hydroxy-2-naphthoic acid, where the acids are employed in salt preparation—depending on whether a mono- or polybasic acid is concerned and depending on which salt is desired—in an equimolar quantitative ratio or one differing therefrom.

On the other hand, salts with bases are—depending on substitution—also suitable. As examples of salts with bases are mentioned the lithium, sodium, potassium, calcium, aluminium, magnesium, titanium, ammonium, meglumine or guanidinium salts, here, too, the bases being employed in salt preparation in an equimolar quantitative ratio or one differing therefrom.

According to the knowledge of the person skilled in the art the acid pump antagonists according to the invention as well as their salts may contain, e.g. when isolated in crystalline form, varying amounts of solvents. Within the scope of the invention the term “acid pump antagonists” includes therefore all solvates and in particular all hydrates of the acid pump antagonists as well as their salts,

In terms of the present invention, as compounds, which modify gastrointestinal motility, active agents from miscallenous active agent classes come into question, such as, for example, the following which are differentiated by modes of action:
5-HT-(partial-)agonists/antagonists (such as, e.g. 5-HT2-, 5-HT3- and 5-HT4-(partial-)agonists/antagonists, in particular 5-HT3-antagonists, 5-HT3-agonists, 5-HT4-agonists, 5-HT4-partial-agonists, 5-HT4-antagonists or dual 5-HT3-antagonists/5-HT4-agonists) known to the person skilled in the art, such as, for example, those mentioned below in the lists 1a, 1b and/or 1c—without being restricted thereto—by means of their INNs or their research code acronyms:
   List 1a comprises and discloses as exemplary 5-HT-(partial-)agonists/antagonists the following active agents:
   (+)-DU-124884, (S)-[125I]-TDP-1040, (S)-[125I]-TDP-960, (S)-[125I]-TDP-984, ADR-851, AU-100, AU-130, AU-224, AU-228, BIMU-1, BIMU-8, BRL-24682, CHF-17454, CILANSETRON, CP-2289, DAZOPRIDE, E-3620, EM-523, FABESETRON, FCE-26778, FCE-27733, FCE-28159, FCE-28232, FCE-28276, FCE-28277, FCE-28278, FCE-28307, FCE-28355, FCE-28356, FCE-28773A, FCE-28797A, FCE-29029A, FCE-29030A, FCE-29031A, FCE-29032A, FCE-29033A, FCE-29034A, KGA-0941, KDR-5169, KF-66854, LINTOPRIDE, LIREXAPRIDE, LY-297524, LY-297582, MOSAPRIDE, PA-6236, PIBOSEROD, PRUCALOPRIDE, PUMOSETRAG, R-76186, RENZAPRIDE, RICASETRON, SB-205149, SB-205800, SB-207710, SC-49518, SC-50410, SC-52246, SC-52491, SC-53116, SC-55822, SC-56184, SK-951, SKF-103829, SKF-47029, SL-90.0629, TEGASEROD, TKS-159, TS-951, VB-20B7, Y-34959, Y-36912, YM-114, YM-47813, YM-47821, YM-53389 and ZACOPRIDE;
   list 1b comprises and discloses as further exemplary 5HT-(partial-)agonists/antagonists the following active agents:
   1192U90, ABAPERIDONE, ADATANSERIN, ALNESPIRONE, ALNIDITAN, ALX-646CL, AMESERGIDE, AR-A000002, ASENAPINE, BEMESETRON, BINOSPIRONE, BLONANSERIN, CERICLAMINE, CILANSETRON, CP-122288, DAZOPRIDE, DOTARIZINE, DU-125530, DULOXETINE, E-2101, E-3620, E-6006, EBALZOTAN, ELZASONAN, EM-523, ENILOSPIRONE, EPLIVANSERIN, FABESETRON, FANANSERIN, FLESINOXAN, FLIBANSERIN, FLUPAROXAN, GEPIRONE, ILOPERIDONE, INDISETRON, IPSAPIRONE, IRINDALONE, IS-159, ITASETRON, LERISETRON, LESOPITRON, LINTOPRIDE, LIREXAPRIDE, LY-353433, LY-53857, MCI-225, MDL-72832, METRENPERONE, MOXIFETIN, ORG-GC-94, OSEMOZOTAN, PALONOSETRON, PELANSERIN, PIBOSEROD, PRUCALOPRIDE, PUMOSETRAG, REC-15/3079, RENZAPRIDE, RICASETRON, RITANSERIN, ROBALZOTAN, ROXINDOLE, RS-25259-197, RU-24969, RUCALOPRIDE, S-15535, SB-243213, SB-271046, SEGANSERIN, SERGOLEXOLE, SKF-38393, SL-65.0155, STACOFYLLINE, T-82, U-93385, VILAZODONE, WAY-100289, XALIPRODEN, Y-36912, YM-114, YM-47813, ZACOPRIDE, ZALOSPIRONE and ZATOSETRON; and
   list 1c comprises and discloses as still further exemplary 5-HT-(partial-)agonists/antagonists the following active agents:
   ALMOTRIPTAN, ALOSETRON, AMPEROZIDE, AZASETRON, BUSPIRONE, CARPIPRAMINE, DEPTROPINE, DIMETOTIAZINE, DOLASETRON, ELETRIPTAN, FLUOXETINE, FROVATRIPTAN, GRANISETRON, LISURIDE, METERGOLINE, MIANSERIN, MOSAPRIDE, NARATRIPTAN, NEFAZODONE, OLANZAPINE, ONDANSETRON, OXITRIPTAN, RAMOSETRON, RISPERIDONE, RIZATRIPTAN, SARPOGRELATE, SERTRALINE, SUMATRIPTAN, TEGASEROD, TROPISETRON, URAPIDIL, ZIPRASIDONE and ZOLMITRIPTAN;
   whereby, in a first facet (facet 1A), exemplary 5-HT-(partial-)agontsts/antagonists according to lists 1a, 1b and 1c more worthy to be mentioned are
   BIMU-1, CILANSETRON, DAZOPRIDE, E-3620, EM-523, FABESETRON, LINTOPRIDE, LIREXAPRIDE, MOSAPRIDE, PIBOSEROD, PRUCALOPRIDE, PUMOSETRAG, R-137696, RENZAPRIDE, RICASETRON, TICALOPRIDE, TEGASEROD, Y-36912, YM-114, YM-47813 and ZACOPRIDE;
   whereby, In a second facet (facet 1B), exemplary 5-HT-(partial-)agonists/antagonists according to lists 1a, 1b and 1c more worthy to be mentioned are
   CILANSETRON, DAZOPRIDE, E-3620, EM-523, FABESETRON, LINTOPRIDE, LIREXAPRIDE, MOSAPRIDE, PIBOSEROD, PRUCALOPRIDE, PUMOSETRAG, RENZAPRIDE, RICASETRON, TEGASEROD, Y-36912, YM-114, YM-47813 and ZACOPRIDE;
   whereby, in the context of facet 1A, exemplary 5-HT-(partial-)agonists/antagonists according to lists 1a, 1b and 1c in particular worthy to be mentioned are
BIMU-1, E-3620, EM-523, LINTOPRIDE, LIREXAPRIDE, PRUCALOPRIDE, MOSAPRIDE, PUMOSETRAG, R-13796, RENZAPRIDE, TICALOPRIDE, TEGASEROD and ZACOPRIDE;
   whereby, in the context of facet 1B, exemplary 5-HT-(partial-)agonists/antagonists according to lists 1a, 1b and 1c in particular worthy to be mentioned are
   E-3620, EM-523, LINTOPRIDE, LIREXAPRIDE, PRUCALOPRIDE, MOSAPRIDE, PUMOSETRAG, RENZAPRIDE, TEGASEROD and ZACOPRIDE;
   and whereby exemplary 5-HT-(partial-)agonists/antagonists according to lists 1a, 1b and 1c in more particular worthy to be mentioned are
   MOSAPRIDE and TEGASEROD;
   and whereby one facet of the class of 5-HT-(partial-)agonists/antagonists comprises
   5-HT2-, 5-HT3- and 5-HT4-(partial-)agonists/antagonists, in particular 5-HT3-antagonists, 5-HT4-agonists, 5-HT4-partial-agonists, or 5-HT4-antagonists;
   and whereby a special subgroup of the class of 5-HT-(partial-)agonists/antagonists comprises those 5-HT-(partial-)agonists/antagonists, which are not either 5-HT4-partial-agonists or 5-HT4-antagonists, and whereby a special subgroup of the class of 5-HT-(partial-)agonists/antagonists to be more emphasized comprises those 5-HT-(partial-)agonists/antagonists mentioned expressis verbis above in the lists 1a, 1b and/or 1c, which are not either 5-HT4-partial-agonists or 5-HT4-antagonists;
   and whereby another special subgroup of the class of 5-HT-(partial-)agonists/antagonists comprises those 5-HT-(partial-)agonists/antagonists, which are not 5-HT4-partial-agonists, 5-HT4-antagonists or dual 5-HT3/5-HT4 agonists/antagonists,
   and whereby another special subgroup of the class of 5-HT-(partial-)agonists/antagonists to be more emphasized comprises those 5-HT-(partial-)agonists/antagonists mentioned expressis verbis above in the lists 1a, 1b and/or 1c, whereof 5-HT4-partial-agonists, 5-HT4-antagonists and dual 5-HT3/5-HT4 agonists/antagonists are disclaimed,
   muscarinic antagonists (e.g. muscarinic M3 antagonists) known to the person skilled in the art, such as, for example, those mentioned below in the lists 2a, 2b and/or 2c—without being restricted thereto—by means of their INNs or their research code acronyms:
      List 2a comprises and discloses as exemplary muscarinic antagonists the following active agents:
      DARIFENACIN and ZAMIFENACIN;
      list 2b comprises and discloses as further exemplary muscarinic antagonists the following active agents:
      (S)-OXYBUTININ, ALVAMELINE, DARENZEPINE, DARIFENACIN, E-6006, FESOTERODINE, KRP-197, KW-5805, OTENZEPAD, REVATROPATE, RISPENZEPINE, SCH-211803, SILTENZEPINE, SINTROPIUM BROMIDE, SOLIFENACIN, TELENZEPINE and VAMICAMIDE; and
      list 2c comprises and discloses as still further exemplary muscarinic antagonists the following active agents:
      PIRENZEPINE, TIOTROPIUM BROMIDE and TOLTERODINE;
      whereby an exemplary muscarinic antagonist according to lists 2a, 2b and/or 2c more worthy to be mentioned is
      DARIFENACIN;
   kappa opioid receptor agonists known to the person skilled in the art, such as, for example, those mentioned below in the lists 3a and/or 3-without being restricted thereto—by means of their INNs or their research code acronyms:
      List 3a comprises and discloses as exemplary kappa oploid receptor agonists the following active agents:
      FEDOTOZINE and ASIMADOLINE; and
      list 3b comprises and discloses as further exemplary kappa oploid receptor agonists the following active agents:
      ADL-10-0101, ADL-10-0116, APADOLINE, ASIMADOLINE, E-2078, ENADOLINE, FEDOTOZINE, IGMESINE, LAPPACONITINE, NALFURAFINE and SPIRADOLINE;
      whereby exemplary kappa opioid receptor agonists according to lists 3a and/or 3b more worthy to be mentioned are
      FEDOTOZINE and ASIMADOLINE;
   delta opioid receptor agonists/antagonists, in particular agonists, known to the person skilled in the art, such as, for example, those mentioned below in the list 4a—without being restricted thereto—by means of their INNs or their research code acronyms:
      list 4a comprises and discloses as exemplary delta oploid receptor agonists the following active agents:
      ALVIMOPAN and TRK-851;
   oploid receptor agonists/antagonists (in particular oploid receptor agonists) known to the person skilled in the art, such as, for example, those mentioned below in the list 5a—without being restricted thereto—by means of their INNs or their research code acronyms:
      List 5a comprises and discloses as exemplary opioid receptor agonists/antagonists the following active agents:
      LEF-553, DIMETHYLTHIAMBUTENE, LOPERAMIDE and REMIFENTANIL;
   dopamine receptor antagonists (in particular dopamine D2 receptor antagonists) known to the person skilled in the art, such as, for example, those mentioned below in the lists 6a, 6b and/or 6c—without being restricted thereto—by means of their INNs or their research code acronyms:
      List 6a comprises and discloses as exemplary dopamine receptor antagonists the following active agents:
      AD-8210, ITOPRIDE, LEVOSULPIRIDE, METOCLOPRAMIDE MOSAPRIDE and TICALOPRIDE;
      list 6b comprises and discloses as further exemplary dopamine receptor antagonists the following active agents:
      1192U90, ABAPERIDONE, BIFEPRUNOX, BLONANSERIN, DAB452, ILOPERIDONE, MAZAPERTINE, RACLOPRIDE, SDZ-GLC-756, SLV-313 and TICALOPRIDE; and
      list 6c comprises and discloses as still further exemplary dopamine receptor antagonists the following active agents:
      ITOPRIDE, LEVOSULPIRIDE, METOCLOPRAMIDE, NEMONAPRIDE, OLANZAPINE, RISPERIDONE, SULPIRIDE and ZIPRASIDONE;
      whereby dopamine receptor agonists according to lists 6a, 6b and/or 6c more worthy to be mentioned are
      ITOPRIDE, LEVOSULPIRIDE, METOCLOPRAMIDE and TICALOPRIDE;
      and whereby dopamine receptor agonists according to lists 6a, 6b and/or 6c in particular worthy to be mentioned are
      ITOPRIDE, LEVOSULPIRIDE and METOCLOPRAMIDE;
   cholecystokinin A antagonists known to the person skilled in the art, such as, for example, those mentioned below in the lists 7a and/or 7b—without being restricted thereto—by means of their INNs or their research code acronyms:
      List 7a comprises and discloses as exemplary cholecystokinin A antagonists the following active agents:
      (−)-RP-73870, (S)-(+)-RP-72540, L-365031 and TARAZEPIDE; and
      List 7b comprises and discloses as further exemplary cholecystokinin A antagonists the following active agents:
      DEVAZEPIDE, DEXLOXIGLUMIDE, KSG-504, LINTITRIPT, LOXIGLUMIDE and PRANAZEPIDE;
   cholecystokinin B antagonists known to the person skilled in the art, such as, for example,
      ITRIGLUMIDE.
   alpha-2 adrenoceptor agonists known to the person skilled in the art, such as, for example, those mentioned below in the list 8a—without being restricted thereto—by means of their INNs or their research code acronyms:
      List 8a comprises and discloses as exemplary alpha-2 adrenoceptor agonists the following active agents:
      ADRAFINIL, APRACLONIDINE, BRIMONIDINE, BUDRALAZINE, CLONIDINE, DEXMEDETOMIDINE, DIMETOFRINE, LOFEXIDINE, MEDETOMIDINE, MOXONIDINE, MPV-295, RILMENIDINE, ROMIFIDINE, S-17089-1, TALIPEXOLE and TIAMENIDINE;
   N-methyl-D-aspartate (NMDA) receptor antagonists known to the person skilled in the art, such as, for example, those mentioned below in the lists 9a and/or 9b—without being restricted thereto—by means of their INNs or their research code acronyms:
      List 9a comprises and discloses as exemplary N-methyl-D-aspartate (NMDA) receptor antagonists the following active agents:
      ACPC, APTIGANEL, BMY-14802, CGP-37849, CNS-5161, DELUCEMINE, DEXANABINOL, DIZOCILPINE, EAA-090, ELIPRODIL, ERLOSAMIDE, FPL-12495, GACYCLIDINE, GAVESTINEL, IPENOXAZONE, LANICEMINE, LICOSTINEL, LIGUSTIZINE, MIDAFOTEL, NERAMEXAN, REMACEMIDE, SELFOTEL, TRAXOPRODIL, UK-240255 and ZD-9379; and
      list 9b comprises and discloses as further exemplary N-methyl-D-aspartate (NMDA) receptor antago nists the following active agents:
      ALIMEMAZINE, AMINOPROMAZINE, CHLORPROETHAZINE, DEXTROMETHORPHAN, FELBAMATE, GLYCINE, MECAMYLAMINE, MILNACIPRAN, PROMAZINE and SERATRODAST;
   non-N-methyl-D-aspartate glutamate receptor antagonists (non-NMDA glutamate receptor antagonists) known to the person skilled in the art, such as, for example, those mentioned below in the list 10a—without being restricted thereto—by means of their INNs or their research code acronyms:
      list 10a comprises and discloses as exemplary non-N-methyl-D-aspartate glutamate receptor antagonists the following active agents:
      FG-9041, FG-9065 and RILUZOLE;
   nitric oxide synthase (NO-synthase) inhibitors known to the person skilled in the art, such as, for example, those mentioned below in the lists 11a and/or 11b—without being restricted thereto—by means of their INNs or their research code acronyms:
      list 11a comprises and discloses as exemplary nitric oxide synthase inhibitors the following active agents:
      CNI-1493, ENECADIN, GW-274150, HP-228, ONO-1714, PIMAGEDINE, TARGININE; and
      list 11b comprises and discloses as further exemplary nitric oxide synthase inhibitor the following active agent:
      TIRILAZAD;
   motilin agonists (motilides) known to the person skilled in the art, such as, for example, those mentioned below in the lists 12a—without being restricted thereto—by means of their INNs or their research code acronyms:
      List 12a comprises and discloses as exemplary motilin agonists the following active agents:
      A-173508, ALEMCINAL, GM-652, GM-665, KC-11458, KW 5139, IDREMCINAL, MITEMCINAL and SK896;
      whereby motilin agonists according to list 12a more worthy to be mentioned are
      ALEMCINAL, IDREMCINAL, MITEMCINAL and SK-896;
   somatostatin agonists/antagonists known to the person skilled in the art, such as, for example, those mentioned below in the list 13a—without being restricted thereto—by means of their INNs or their research code acronyms:
      List 13a comprises and discloses as exemplary somatostatin agonists/antagonists the following active agents:
      L-054852, OCTREOTIDE, VAPREOTIDE and LANREOTIDE;
   neurotensin (partial) agonists/antagonists (suitably neurotensin agonists) known to the person skilled in the art, such as, for example, those mentioned below in the lists 14a—without being restricted thereto—by means of their INNs or their research code acronyms:
      List 14a comprises and discloses as exemplary neurotensin (partial) agonists/antagonists the following active agents:
      SR-142948-A, REMINERTANT and, suitably, CONTULAKIN G, CITRULLIMYCINE A and NT69L;
   vasoactive intestinal peptide (VIP) antagonists known to the person skilled in the art, such as, for example, this mentioned below in the list 15a—without being restricted thereto—by means of its research code acronym:
      List 15a comprises and discloses as an exemplary vasoactive intestinal peptide antagonist the following active agent:
      RO-25-1553;
   substance P(SP) antagonists known to the person skilled in the art, such as, for example, NK-1 antagonists and/or in particular those antagonists mentioned below in the list 16a—without being restricted thereto—by means of their INNs or their research code acronyms:
      List 16a comprises and discloses as exemplary substance P antagonists the following active agents:
      CGP-49823, EZLOPITANT and LANEPITANT;
   neurokinin antagonists (in particular NK-1, NK-2 or NK-3 antagonists) known to the person skilled in the art, such as, for example, those NK-1 anatgonists, which are disclosed in the international application WO 0069438 as useful to be employed in combination therapy, and/or in particular those neurokinin antagonists mentioned below in the list 17a—without being restricted thereto—by means of their INNs or their research code acronyms:
      List 17a comprises and discloses as exemplary neurokinin antagonists the following active agents:
      ALTINICLINE, APREPITANT, CGP-49823, CP-122721, EZLOPITANT as selective NK-1 antagonist, NEPADUTANT as selective NK-2 antagonist, LANEPITANT, OSANETANT, S-19752, SAREDUTANT, TALNETANT and VOFOPITANT,
      whereby neurokinin antagonists according to list 17a more worthy to be mentioned are
      NEPADUTANT, SAREDUTANT or TALNETANT;
   calcium channel blockers known to the person skilled in the art, such as, for example, those mentioned below in the lists 18a and/or 18b—without being restricted thereto—by means of their INNs or their research code acronyms:
      List 18a comprises and discloses as exemplary calcium channel blockers the following active agents:
      AZELNIDIPINE, BELFOSDIL, BISARAMIL, CD-832, CERM-11956, CLENTIAZEM, CRE-202, CRONIDIPINE, CV-159, DAURICINE, DHP-218, DIPERDIPINE, DIPROTEVERINE, DOPROPIDIL, DOTARIZINE, ELGODIPINE, EMOPAMIL, FANTOFARONE, FOSTEDIL, FPL-62129, FURNIDIPINE, HA-1004, IGANIDIPINE, IOS-11212, KT-362, LECONOTIDE, LEMILDIPINE, LIFARIZINE, LUBELUZOLE, MANOALIDE, MCN-5691, MEPAMIL, MIOFLAZINE, MONATEPIL, NICTIAZEM, OLRADIPINE, OXODIPINE, P-0285, PRANIDIPINE, RANOLAZINE, RIODIPINE, RONIPAMIL, S-312, SABELUZOLE, SB-237376, SEMOTIADIL, SL-87.0495, SQ-31765, TAMOLARIZINE, TIPROPIDIL, TROMBODIPINE, VATANIDIPINE, YM-16151-4 and ZICONOTIDE; and
      list 18b comprises and discloses as further exemplary calcium channel blockers the following active agents:
      AMLODIPINE, ARANIDIPINE, BARNIDIPINE, BENCYCLANE, BENIDIPINE, BEPRIDIL, BUFLOMEDIL, CAROVERINE, CILNIDIPINE, CINNARIZINE, DILTIAZEM, DROPRENILAMINE, EFONIDIPINE, FASUDIL, FELODIPINE, FENDILINE, FLUNARIZINE, GALLOPAMIL, ISRADIPINE, LACIDIPINE, LERCANIDIPINE, LIDOFLAZINE, LOMERIZINE, MANIDIPINE, NADOLOL, NICARDIPINE, NIFEDIPINE, NILVADIPINE, NIMODIPINE, NISOLDIPINE, NITRENDIPINE, PERHEXILINE, PINDOLOL, TERODILINE and VERAPAMIL;
   potassium channel openers known to the person skilled in the art, such as, for example, those menboned below in the lists 19a and/or 19b—without being restricted thereto—by means of their INNs or their research code acronyms:
      List 19a comprises and discloses as exemplary potassium channel openers the following active agents:
      ABT-598, APRIKALIM, BIMAKALIM, EMAKALIM, EMD-57283, FLINDOKALNER, KCO-912, KRN-2391, LEMAKALIM, LEVCROMAKALIM, NN-414, NS-8, RETIGABINE, RP-49356, Y-27152, ZD-0947 and ZD-6169; and
      list 19b comprises and discloses as further exemplary potassium channel openers the following active agents:
      LEVOSIMENDAN, NICORANDIL and PINACIDIL;
   selective serotonin reuptake inhibitors (SSRIs) known to the person skilled in the art, such as, for example, those mentioned below in the lists 20a and/or 20b—without being restricted thereto—by means of their INNs or their research code acronyms:
      List 20a comprises and discloses as exemplary selective serotonin reuptake inhibitors the following active agents:
      BROFAROMINE, BTS-74398, CERICLAMINE, CYANODOTHIEPIN, DELUCEMINE: DULOXETINE, LU-35-138, LUBAZODONE, MANIFAXINE and VILAZODONE; and
      list 20b comprises and discloses as further exemplary selective serotonin reuptake inhibitors the following active agents:
      CITALOPRAM, ESCITALOPRAM, FLUOXETINE, FLUVOXAMINE, MILNACIPRAN, NEFAZODONE, PAROXETINE, SERTRALINE and VENLAFAXINE;
   corticotropin releasing factor antagonists known to the person skilled in the art, such as, for example, this mentioned below in the list 21a—without being restricted thereto—by means of its INN:
      List 21a comprises and discloses as exemplary corticotropin releasing factor antagonists the following active agent:
      ANTALARMIN or SB-723620;
   agonists of gamma-aminobutyric acid receptors of the A-typ (GABA-A receptor agonists) known to the person skilled in the art, such as, for example, those mentioned below in the list 22a—without being restricted thereto—by means of their INNs or their research code acronyms:
      List 22a comprises and discloses as exemplary GABA-A receptor agonists the following active agents:
      GABOXADOL, GEDOCARNIL, ORG-25435, PAGOCLONE and RETIGABINE;
   agonists/partial agonists of gamma-aminobutyric acid receptors of the B-typ (GABA-B receptor agonists/partial agonists) known to the person skilled in the art, such as, for example, those mentioned below in the lists 23a and/or 23b, without being restricted thereto:
      List 23a comprises and discloses as exemplary GABA-B receptor agonists the following active agents:
      AZD-3355, BACLOFEN (in more detail (±)-baclofen, S(−)-baclofen or R(+)-baclofen), GABAPENTIN, PAZINACLONE, CGP-29030A, CGP44532, SL-65.1498 and SKF-97541;
      and those which are disclosed in WO 9811885, EP 0356128, EP 0181833, EP 0399949, EP 0463969, FR 2,722,192 or in J. Med. Chem (1995), 38, 3297-3312 (such as, e.g. (S)-(3-amino-2-hydroxypropyl)methylphosphinic acid);
      and those which are named expressis verbis (e.g. as an example) or described and/or claimed generically in WO 02100823, WO 02100869, WO 02100870 or WO 02100871 such as, for example,
4-amino-3-phenylbutanoic acid,
4-amino-3-hydroxybutanoic acid,
4-amino-3-(4-chlorophenyl)-3-hydroxyphenylbutanoic acid,
4-amino-3-(thien-2-yl)butanoic add,
4-amino-3-(5-chlorothien-2-yl)butanoic acid,
4-amino-3-(5-bromothien-2-yl)butanoic add,
4-amino-3-(5-methylthien-2-yl)butanoic acid,
4-amino-3-(2-imidazolyl)butanoic acid,
4-guanidino-3-(4-chlorophenyl)butanoic acid,
3-amino-2-(4-chlorophenyl)-l-nitropropane,
(3-aminopropyl)phosphonous acid,
(4-aminobut-2-yl)phosphonous acid,
(3-amino-2-methylpropyl)phosphonous acid,
(3-aminobutyl)phosphonous acid,
(3-amino-2-(4-chlorophenyl)propyl)phosphonous acid,
(3-amino-2-4-chlorophenyl)-2-hydroxypropyl)phosphonous acid,
(3-amino-2-(4-fluorophenyl)propyl)phosphonous acid,
(3-amino-2-phenylpropyl)phosphonous acid,
(3-amino-2-hydroxypropyl)phosphonous acid,
(E)-(3-aminopropen-1-yl)phosphonous acid,
(3-amino-2-cyclohexylpropyl)phosphonous acid,
(3-amino-2-benzylpropyl)phosphonous acid,
[3-amino-2-(4-methylphenyl)propyl]phosphonous acid,
[3-amino-2-(4-trifluoromethylphenyl)propyl]phosphonousacid,
[3-amino-2-(4-methoxyphenyl)propyl]phosphonous acid,
(3-amino-2-(4-chlorophenyl)-2-hydroxypropyl]phosphonousacid,
(3-aminopropyl)methylphosphinic acid,
(3-amino-2-hydroxypropyl)methylphosphinic acid,
(3-aminopropyl)(difluoromethyl)phosphinic acid,
(4-aminobut-2-yl)methylphosphinic acid,
(3-amino-1-hydroxypropyl)methylphosphinic acid,
(3-amino-2-hydroxypropyl)(difluoromethyl)phosphinic acid,
(E)-(3-aminopropen-1-yl)methylphosphinic acid,
(3-amino-2-oxo-propyl)methyl phosphinic acid,
(3-aminopropyl)hydroxymethylphosphinic acid,
(5-aminopent-3-yl)methylphosphinic acid,
(4-amino-1,1,1-trifluorobut-2-yl)methylphosphinic acid,
(3-amino-2-(4-chlorophenyl)propyl)sulfinic acid or
3-aminopropylsulfinic acid
   or a pharmaceutically acceptable salt, solvate or stereoisomer thereof;
   whereby, in a first facet, exemplary GABA-B agonists according to list 23a more worthy to be mentioned are
   GABAPENTIN, BACLOFEN, PAZINACLONE and SL-65.1498;
   whereby, in a second facet, exemplary GABA-B agonists according to list 23a more worthy to be mentioned are
   GABAPENTIN, BACLOFEN, PAZINACLONE, CGP-29030A and SL-65.1498;
   and whereby exemplary GABA-B agonists according to list 23a in particular worthy to be mentioned are
   GABAPENTIN and BACLOFEN.

List 23b comprises and discloses as exemplary GABA-B receptor agonists the following active agents: those GABA-B receptor agonists which are named expressis verbis od described and/or claimed generically in WO2004/000855 and/or WO2004/000866 such as, for example,
(3-amino-2-fluoropropyl)phosphinic acid,
(R)-(3-amino-2-fluoropropyl)phosphinic acid,
(S)-(3-amino-2-fluoropropyl)phosphinic acid,
(3-amino-2-fluoro-1-methyl-propyl)phosphinic acid,
(3-amino-2-oxopropyl)phosphinic acid,
(S)-(3-amino-2-hydroxypropyl)phosphinic acid,
(R)-(3-amino-2-hydroxypropyl)phosphinic acid,
(3-amino-1-fluoro-2-hydroxypropyl)phosphinic acid,
(3-amino-2-fluoro-propyl)(methyl)phosphinic acid,
(2R)-(3-amino-2-fluoro-propyl)(methyl)phosphinic acid,
(2S)-(3-amino-2-fluoro-propyl)(methyl)phosphinic acid,
(3-amino-2-fluoro-1-methylpropyl)(methyl)phosphinic acid,
(3-amino-1-fluoropropyl)phosphinic acid,
3-[(4-chlorobenzyl)amino]propyl(methyl)phosphinic acid,
3-[1-({3-[hydroxy(oxido)phosphino]propyl}amino)ethyl]benzoic acid acid,
(3-amino-2-fluoropropyl)sulphinic acid,
(2S)-(3-amino-2-fluoropropyl)sulphinic acid,
(2R)-(3-amino-2-fluoropropyl)sulphinic acid,
(2S)-(3-amino-2-hydroxypropyl)sulphinic acid,
(2R)-(3-amino-2-hydroxypropyl)sulphinic acid, or
(3-amino-2-oxopropyl)sulphinic acid,
   or a pharmaceutically acceptable salt, solvate or stereoisomer thereof.

In the context thereof, AZD-3355 and AZD-9343 are to be mentioned in an independent embodimental aspect.

In addition to the specification given above, the term “compounds, which modify gastrointestinal motility” also comprises in the meaning of the present invention active agents from the following active agent classes which are—in contrast to the above differentiation by modes of action—now differentiated by physiological effects:
gastroprokinetics known to the person skilled in the art, such as, for example, those mentioned below in the list 24a and/or, advantageously, in the lists 24b and/or 24c—without being restricted thereto—by means of their INNs or their research code acronyms:
   List 24a comprises and discloses as exemplary gastroprokinetics the following active agents:
   *243740, A-124728, ALFA-604, CHIR-6028, CYCRIMINE, DOBUPRIDE, EM-536, FLUPERAMIDE, KW-5092, KW-5139, L-368935, L-369466, LOPERAMIDE, P-1380, R-137696, R-18936, RP-73870, SILDENAFIL, SKF-91606, SLV-305, SR-58339, SR-58375-A, SR-58611-A, SR-58878, T-1815, TRIPERIDEN, YM-31636;
   list 24b comprises and discloses as further exemplary gastroprokinetics the following active agents:
   ALEMCINAL, DARIFENACIN, DOBUPRIDE, E-3620, EM-523, FEDOTOZINE, IDREMCINAL, KW-5092, KW-5139, LINTOPRIDE, LIREXAPRIDE, MITEMCINAL, NITRAQUAZONE, PUMOSETRAG, PRUCALOPRIDE, R-137696, RENZAPRIDE, ROLIPRAM, SK-896, SR-58611-A, T-1815, TIBENELAST, TICALOPRIDE, Z-338, ZACOPRIDE; and
   list 24c comprises and discloses as still further exemplary gastroprokinetics the following active agents:
   BIPERIDEN, BUDIPINE, CINITAPRIDE, FEDOTOZINE, ITOPRIDE, LOPERAMIDE, PROCYCLIDINE, SULTOPRIDE, TEGASEROD and TRIHEXYPHENIDYL;
   whereby gastroprokinetics according to lists 24a, 24b and 24c more worthy to be mentioned are ALEMCINAL, CINITAPRIDE, DOBUPRIDE, FEDOTOZINE, KW-5092, KW-5139, ITOPRIDE, LIREXAPRIDE, MITEMCINAL, PRUCALOPRIDE, R-137696, RENZAPRIDE, SR-58611-A, T-1815, TEGASEROD, TICALOPRIDE and Z-338; and
   whereby gastroprokinetics according to lists 24a, 24b and 24c in particular worthy to be mentioned are
   CINITAPRIDE, ITOPRIDE and TEGASEROD;
antiemetics known to the person skilled in the art, such as, for example, those mentioned below in the lists 25a and/or, advantageously, in the lists 25b, 25c and/or 25d—without being restricted thereto
by means of their INNs or their research code acronyms:
   List 25a comprises and discloses as exemplary antiemetics the following active agents:
   CINITAPRIDE, RENZAPRIDE and TICALOPRIDE;
   list 25b comprises and discloses as further exemplary antiemetics the following active agents:
   AD-8210, ADR-847, ADR-851, BRL-20627-A, BRL-24682, PA-6236, R-51430 and SL-90.0629;
   list 25c comprises and discloses as still further exemplary antiemetics the following active agents:
   ALTINICLINE, APREPITANT, BATANOPRIDE, CILANSETRON, DAZOPRIDE, DEXANABINOL, E-3620, EXEPANOL, FABESETRON, INDISETRON, ITASETRON, LERISETRON, LINTOPRIDE, PALONOSETRON, RS-25259-197, VOFOPITANT and ZACOPRIDE;
   list 25d comprises and discloses as also still further exemplary antiemetics the following active agents:
   ACETYLLEUCINE, ALIZAPRIDE, ALOSETRON, AZASETRON, BROMOPRIDE, CISAPRIDE, CLEBOPRIDE, DIFENIDOL, DOMPERIDONE, DRONABINOL, GRANISETRON, LEVOSULPIRIDE, METOCLOPRAMIDE, MOSAPRIDE, ONDANSETRON, OXYPENDYL, RAMOSETRON, THIETHYLPERAZINE, TIAPRIDE, TRIMETHOBENZAMIDE and TROPISETRON;
   whereby antiemetics according to lists 25a, 25b, 25c and 25d more worthy to be mentioned are CINITAPRIDE, RENZAPRIDE, TICALOPRIDE and, especially,
   CISAPRIDE, CLEBOPRIDE, DIFENIDOL, E-3620, LEVOSULPIRIDE, LINTOPRIDE, METOCLOPRAMIDE, MOSAPRIDE and ZACOPRIDE;
   and whereby antiemetics in particular worthy to be mentioned are
   CINITAPRIDE and, especially,
   CISAPRIDE, CLEBOPRIDE, DIFENIDOL, LEVOSULPIRIDE, METOCLOPRAMIDE and MOSAPRIDE;
antispasmodics (for example anticholinergics or smooth muscle relaxants) known to the person skilled in the art, such as, for example, those mentioned below in the list 26a—without being restricted thereto—by means of their INNs or their research code acronyms:
   List 26a comprises and discloses as exemplary antispasmodics the following active agents:
   CIMETROPIUM BROMIDE, BIPERIDEN, DENBUFYLLINE, ETAZOLATE, FETOXILATE, ICI-63197, MEBEVERINE, NITRAQUAZONE, ORG-30029, PINAVERIUM BROMIDE, PRIDINOL, PROCYCLIDINE, ROLIPRAM, TIBENELAST, TRIHEXYPHENIDYL, TRIMEBUTINE, UK84149 and ZARDAVERINE;
   whereby antispasmodics according to list 26a more worthy to be mentioned are
   BIPERIDEN, PRIDINOL, PROCYCLIDINE, NITRAQUAZONE, ROLIPRAM, TRIHEXYPHENIDYL, TIBENELAST and, especially, MEBEVERINE;
   and whereby antispasmodics according to list 26a in particular worthy to be mentioned are
   BIPERIDEN, PRIDINOL, PROCYCLIDINE, TRIHEXYPHENIDYL and, especially, MEBEVERINE.

A person of ordinary skill in the art knows that the abovementioned classification of the specific active agents in said active agent classes should not be regarded in a strict, sole or exclusive meaning. On the contrary, certain active agents can be allocated to more than one active agent class given above, in particular certain active agents can be allocated both to one or more of the abovementioned active agent classes differentiated by modes of action and to one or more of the abovementioned active agent classes differentiated by physiological effects.

Within the scope of this invention, the term “compounds, which modify gastrointestinal motility” comprises not only the active compounds or active agents per se but also pharmacologically acceptable derivatives such as, for example, pharmaceutically acceptable salts, solvates (in particular hydrates), solvates of the salts, polymorphs, tautomers, racemates, diastereoisomers or enantiomers of these compounds or agents.

In the meaning of the present invention, a first special aspect (aspect a) of the term “compounds, which modify gastrointestinal motility” refers to those compounds, which modify gastrointestinal motility and reduce the incidence of transient lower esophageal sphincter relaxation (TLOSR). As exemplary compounds according to aspect a, neurokinin-1 (NK-1) antagonists and, particularly, GABA-B receptor agonists/partial agonists are to be mentioned, in particular those specified above by reference or expresses verbis. Exemplary compounds, which reduce the incidence of transient lower esophageal sphincter relaxation (TLOSR), according to aspect a to be emphasized are, in one facet, 4-amino-3-(4-chlorophenyl)butanoic acid (baclofen), (3-aminopropyl)methylphosphinic acid, (3-amino-2-hydroxypropyl)methylphosphinic acid, (3-amino-2-(4-chlorophenyl)propyl)sulfinic acid, (3-aminopropyl)(difluoromethyl)phosphinic acid, (3-amino-2-oxo-propyl)methyl phosphinic acid, 4-amino-3-(5-chlorothien-2-yl)butanoic acid and (3-aminopropyl)phosphonous acid, or, in another facet, the compounds mentioned in list 23b.

A second special aspect (aspect b) of the term “compounds, which modify gastrointestinal motility” refers to those compounds, which modify gastrointestinal motility, and, which are particularly useful for therapy of irritable bowel syndrome (IBS), such as, for example, those compounds of the following active agent classes:

5-HT-(partial-)agonists/antagonists (such as, e.g. 5-HT3-antagonists, 5-HT3-agonists, 5-HT4-agonists,

5-HT4-partial-agonists, 5-HT4-antagonists or dual 5-HT3-antagonists/5-HT4-agonists), cholecystokinin A antagonists, muscarinic M3 antagonists, kappa opioid receptor agonists, motilin agonists (motilides), delta opioid receptor agonists, dopamine receptor antagonists, neurokinin antagonists (in particular NK-1, NK-2 or NK-3 antagonists), NMDA-receptor antagonists, alpha-2 adrenoceptor agonists or corticotropin releasing factor antagonists,

whereby

5-HT-(partial-)agonists/antagonists (such as, e.g. 5-HT3-antagonists, 5-HT4-agonists, 5-HT4-partial-agonists, 5-HT4-antagonists), cholecystokinin A antagonists, muscarinic M3 antagonists, kappa opioid receptor agonists, motilin agonists (motilides), delta opioid receptor agonists and dopamine receptor antagonists are more worthy to be mentioned,

or whereby, in an alternative,

5-HT-(partial-)agonists/antagonists (such as, e.g. 5-HT3-antagonists, 5-HT4-agonists, 5-HT4-partial-agonists, 5-HT4-antagonists or dual 5-HT3-antagonists/5-HT4-agonists), cholecystokinin A antagonists, neurokinin antagonists, muscarinic M3 antagonists, or kappa or delta opioid receptor agonists are more worthy to be mentioned,

or whereby, in another alternative,

5-HT-(partial-)agonists/antagonists (such as, e.g. 5-HT3-antagonists, 5-HT4-agonists, 5-HT4-partial-agonists, 5-HT4-antagonists or dual 5-HT3-antagonists/6-HT4-agonists) are further more worthy to be mentioned.

As exemplary compounds according to said special aspect b are to be mentioned, in one facet, without being restricted thereto,

CLONIDINE (as exemplary alpha-2 adrenoceptor agonist), DIZOCILPINE (as exemplary NMDA-receptor antagonist), EZLOPITANT (as exemplary selective NK-1 antagonist), NEPADUTANT (as exemplary selective NK-2 antagonist), ANTALARMIN (as exemplary corticotropin releasing factor antagonist) and, in particular,

CILANSETRON, DARIFENACIN, E-3620, FABESETRON, LINTOPRIDE, LY-353433, (S)-OXYBUTININ, TICALOPRIDE, ZAMIFENACIN and, in more particular,

ALOSETRON, TRIMEBUTINE, TEGASEROD and, in further more particular,

ALVIMOPAN, DEXLOXIGLUMIDE and PIBOSEROD.

As exemplary compounds according to the active agent classes of said special aspect b are to be mentioned, in another facet, without being restricted thereto,

those compounds specified in this invention as exemplary compounds of this active agent classes given above in aspect b.

As exemplary compounds according to said special aspect b are to be mentioned, in yet another facet, without being restricted thereto,

YM-114, FABESETRON, E-3620, LY-353433, TICALOPRIDE, PRUCALOPRIDE, PIBOSEROD, CILANSETRON, ALOSETRON, TEGASEROD, RAMOSETRON,

DEXLOXIGLUMIDE,

NEPADUTANT, SAREDUTANT, TALNETANT,

FEDOTOZINE, PTI-901, ASIMADOLINE,

ALVIMOPAN,

(S)-OXYBUTININ, J-104135, DARIFENAZIN or ZAMIFENACIN.

As a more precisely facet of special aspect b, the 5-HT-(partial-)agonist/antagonist class is to be mentioned including for example, without being restricted thereto, the following compounds:

YM-114, FABESETRON, E-3620, LY-353433, TICALOPRIDE, or, in particular, PRUCALOPRIDE, PIBOSEROD or CILANSETRON, or, in more particular, ALOSETRON or TEGASEROD,

or, in a more detailed alternative,

5-HT4 antagonists such as e.g.: PIBOSEROD, or LY-353433,

5-HT3 antagonists such as e.g.: YM-114, or CILANSETRON, RAMOSETRON or ALOSETRON,

5-HT4 partial agonists such as e.g.: TEGASEROD,

5-HT4 agonists such as e.g.: PRUCALOPRIDE,

dual 5-HT3 antagonist/5-HT4 agonists such as e.g.: FABESETRON, or E-3620 or RENZAPRIDE.

As another more precisely facet of special aspect b, the cholecystokinin A antagonist class is to be mentioned including for example, without being restricted thereto, the following compounds:

DEXLOXIGLUMIDE.

As another more precisely facet of special aspect b, the neurokinin antagonist class is to be mentioned including for example, without being restricted thereto, the following compounds:

NK-2 antagonists such as e.g.: NEPADUTANT or SAREDUTANT,

NK-3 antagonists such as e.g.: TALNETANT.

As another more precisely facet of special aspect b, the kappa opioid receptor agonist class is to be mentioned including for example, without being restricted thereto, the following compounds:

FEDOTOZINE, PTI-901 or, particularly, ASIMADOLINE.

As another more precisely facet of special aspect b, the delta opioid receptor agonist class is to be mentioned including for example, without being restricted thereto, the following compounds:

ALVIMOPAN.

As another more precisely facet of special aspect b, the muscarinic, in particular muscarinic M3, antagonist class is to be mentioned including for example, without being restricted thereto, the following compounds:

ZAMIFENACIN, or (S)-OXYBUTININ, J-104135 or DARIFENAZIN.

As an in particular precisely facet of special aspect b, TEGASEROD is to be mentioned.

As another in particular precisely facet of special aspect b, ALOSETRON is to be mentioned.

As another in particular precisely facet of special aspect b, CILANSETRON is to be mentioned.

As another in particular precisely facet of special aspect b, PRUCALOPRIDE is to be mentioned.

As another in particular precisely facet of special aspect b, ALVIMOPAN is to be mentioned.

As another in particular precisely facet of special aspect b, PIBOSEROD is to be mentioned.

As another in particular precisely facet of special aspect b, DEXLOXIGLUMIDE is to be mentioned.

A third special aspect (aspect c) of the term “compounds, which modify gastrointestinal motility” refers to those compounds, which modify gastrointestinal motility, and, which are particularly useful for therapy of gastro-esophageal reflux disease (GERD), such as, for example, compounds of the class of motilin agonists (mobilides), of the class of 5-HT-(partial-)agonists/antagonists (such as, e.g. 5-HT3-antagonists, 5-HT3-agonists, 5-HT4-agonists, 5-HT4-partial-agonists, 5-HT4-antagonists, or dual 5-HT3-antagonists/5-HT4-agonists), of the class of muscarinic antagonists, of the class of opioid agonists/partial agonists, of the class of NMDA receptor antagonists, of the class of non-NMDA glutamate receptor antagonists, of the class of somatostatin agonists, of the class of NO-synthase inhibitors, of the class of GABA (in particular GABA-B) receptor agonists or active agents which reduce the incidence of transient lower esophageal sphincter relaxation (TLOSR),

whereby

compounds of the class of motilin agonists (motilides), of the class of 5-HT-(partial-)agonists/antagonists (such as, e.g. 5-HT3-antagonists, 5-HT4-agonists, 5-HT4-partial-agonists, 5-HT4-antagonists), of the class of GABA-B receptor agonists or active agents which reduce the incidence of transient lower esophageal sphincter relaxation (TLOSR) are more worthy to be mentioned, or whereby, in an alternative,

5-HT-(partial-)agonists/antagonists (such as, e.g. 5-HT3-antagonists, 5-HT3-agonists, 5-HT4-agonists,

5-HT4-partial-agonists, 5-HT4-antagonists or dual 5-HT3-antagonists/5-HT4-agonists), motilin agonists, cholecystokinin A or B antagonists, dopamine antagonists,

GABA-B receptor agonists or active agents which reduce the incidence of transient lower esophageal sphincter relaxation (TLOSR) are more worthy to be mentioned,

or whereby, in a further alternative,

5-HT-(partial-)agonists/antagonists (such as, e.g. 5-HT3-antagonists, 5-HT3-agonists, 5-HT4-agonists,

5-HT4-partial-agonists, 5-HT4-antagonists or dual 5-HT3-antagonists/5-HT4-agonists), are further more worthy to be mentioned.

As exemplary compounds according to said special aspect c are to be mentioned, in one facet, without being restricted thereto,

PIBOSEROD, MITEMCINAL and, particularly,

TEGASEROD.

As exemplary compounds according to the active agent classes of said special aspect c are to be mentioned, in another facet, without being restricted thereto,

those compounds specified in this invention as exemplary compounds of this active agent classes given above in aspect c.

As exemplary compounds according to said special aspect c are to be mentioned, in yet another facet, without being restricted thereto,

TICALOPRIDE, TEGASEROD, PIBOSEROD, MOSAPRIDE, PUMOSETRAG,

MITEMCINAL,

ITRIGLUMIDE, Z-360, or

DEXLOXIGLUMIDE.

As a more precisely facet of special aspect c, the 5-HT-(partial-)agonist/antagonist class (such as, e.g. 5-HT3-antagonists, 5-HT4-agonists, 5-HT4-partial-agonists, 5-HT4-antagonists, 5-HT3-agonists, or dual 5-HT3-antagonists/5-HT4-agonists) is to be mentioned including for example, without being restricted thereto, the following compounds: TICALOPRIDE,

or, in a more detailed alternative,

5-HT4 partial agonists such as e.g.: TEGASEROD,

5-HT4 antagonists such as e.g.: PIBOSEROD,

5-HT4 agonists such as e.g.: MOSAPRIDE,

5-HT3-agonists such as e.g.: PUMOSETRAG.

As another more precisely facet of special aspect c, the motilin receptor agonist class is to be mentioned including for example, without being restricted thereto, the following compounds:

MITEMCINAL.

As another more precisely facet of special aspect c, the cholecystokinin B antagonist class is to be mentioned including for example, without being restricted thereto, the following compounds:

ITRIGLUMIDE, or Z-360.

As another more precisely facet of special aspect c, the cholecystokinin A antagonist class is to be mentioned including for example, without being restricted thereto, the following compounds:

DEXLOXIGLUMIDE.

As another more precisely facet of special aspect c, the class of active agents which reduce the incidence of transient lower esophageal sphincter relaxation (TLOSR) is to be mentioned including for example, without being restricted thereto, the compounds mentioned herein and/or the following compounds:

GABA-B receptor agonists such as e.g. those mentioned in the specification of this invention.

As an in particular precisely facet of special aspect c, TEGASEROD is to be mentioned.

A fourth special aspect (aspect d) of the term “compounds, which modify gastrointestinal motility” refers to those compounds, which modify gastrointestinal motility, and, which are particularly useful antiemetics, such as, for example, compounds of the class of

5-HT-(partial-)agonists/antagonists (such as, e.g. 5-HT3-antagonists, 5-HT4-agonists, 5-HT4-partial-agonists or 5-HT4-antagonists), of the class of dopamine receptor antagonists (in particular dopamine D2 receptor antagonists), of the class of NMDA receptor antagonists or of the class of neurokinin antagonists (in particular NK-1, NK-2 or NK-3 antagonists).

As exemplary compounds according to said special aspect d are to be mentioned, without being restricted thereto,

ALTINICLINE, APREPITANT, BATANOPRIDE, CILANSETRON, DAZOPRIDE, DEXANABINOL, E-3620, EXEPANOL, FABESETRON, INDISETRON, ITASETRON, LERISETRON, LINTOPRIDE, PALONOSETRON, RS-25259-197, VOFOPITANT, ZACOPRIDE and, particularly,

ALIZAPRIDE, ALOSETRON, AZASETRON, BROMOPRIDE, CISAPRIDE, CLEBOPRIDE, DIFENIDOL, DOMPERIDONE, GRANISETRON, LEVOSULPIRIDE, METOCLOPRAMIDE, MOSAPRIDE, ONDANSETRON, RAMOSETRON, TIAPRIDE and TROPISETRON.

A fifth special aspect (aspect e) of the term “compounds, which modify gastrointestinal motility” refers to those compounds, which modify gastrointestinal motility, and, which are particularly useful gastroprokinetics, such as, for example, compounds of the class of

5-HT-(partial-)agonists/antagonists (such as, e.g. 5-HT2-, 5-HT3- and 5-HT4-(partial-)agonists/antagonists), muscarinic antagonists, kappa oploid receptor agonists, dopamine receptor antagonists (in particular dopamine D2 receptor antagonists), cholecystokinin A antagonists, motilin agonists (motilides) or GABA-B receptor agonists/partial agonists, or whereby, in an alternative,

5-HT-(partial-)agonists/antagonists (such as, e.g. 5-HT3-antagonists, 5-HT3-agonists, 5-HT4-agonists,

5-HT4-partial-agonists, 5-HT4-antagonists or dual 5-HT3-antagonists/5-HT4-agonists), motilin agonists, opioid receptor agonists or dopamine receptor antagonists are also to be mentioned,

or whereby, in a further alternative,

motilin receptor agonists such as e.g.: ALEMCINAL, or MITEMCINAL,

5-HT-(partial-)agonist/antagonists such as e.g.: LIREXAPRIDE,

dopamine D2 receptor anatgonists such as e.g.: TICALOPRIDE, or ITOPRIDE,

5-HT4 partial agonists such as e.g.: TEGASEROD,

5-HT4 agonists such as e.g.: PRUCALOPRIDE,

kappa opioid receptor agonists such as e.g.: FEDOTOZINE, are also to be mentioned.

As exemplary compounds according to said special aspect e are to be mentioned in one facet, without being restricted thereto,

ALEMCINAL, DOBUPRIDE, FEDOTOZINE, KW-5092, KW-5139, LIREXAPRIDE, PRUCALOPRIDE, R-137696, RENZAPRIDE, SR-58611-A, Z-338 and, in particular,

MITEMCINAL, TICALOPRIDE,

and, in more particular,

CINITAPRIDE, ITOPRIDE and, in most particular,

TEGASEROD.

As exemplary compounds according to the active agent classes of said special aspect e are to be mentioned, in another facet, without being restricted thereto,

those compounds specified in this invention as exemplary compounds of this active agent classes given above in aspect e.

As exemplary compounds according to said special aspect e are to be mentioned in yet another facet, without being restricted thereto,

ALEMCINAL, BIMU-1, DOBUPRIDE, FEDOTOZINE, KW-5092, KW-5139, LIREXAPRIDE, PRUCALOPRIDE, R-137696, RENZAPRIDE, SR-58611-A, T-1815, Z-338, MITEMCINAL, TICALOPRIDE, CINITAPRIDE, ITOPRIDE or TEGASEROD.

A sixth special aspect (aspect f) of the term “compounds, which modify gastrointestinal motility” refers to those compounds, which are selected from

the class of 5-HT-(partial-)agonists/antagonists (such as, e.g. 5-HT2-, 5-HT3- and 5-HT4-(partial-)agonists/antagonists, in particular 5-HT3-antagonists, 5-HT4-agonists, 5-HT4-partial-agonists, 5-HT4-antagonists or dual 5-HT3-antagonists/5-HT4-agonists), from the class of muscarinic antagonists, from the class of kappa opioid receptor agonists, from the class of dopamine receptor antagonists (in particular dopamine D2 receptor antagonists), from the class of cholecystokinin A antagonists, from the class of motilin agonists (motilides) or from the class of GABA-B receptor agonists/partial agonists or from active agents which reduce the incidence of transient lower esophageal sphincter relaxation (TLOSR).

A seventh special aspect (aspect g) of the term “compounds, which modify gastrointestinal motility” refers to those compounds, which are selected from

the class of 5-HT-(partial-)agonists/antagonists.

A first subaspect of the the expression “5-HT-(partial-)agonists/antagonists” according to said special aspect g refers to 5-HT4-partial-agonists. In this context, 5-HT4-partial-agonists include any compound which can partially activate 5-HT4 receptors (intrinsic activity less than that of serotonin, i.e. <1.00. The intrinsic activity may be determined in the non-electrically or electrically stimulated guinea pig ileum or striatum assay, e.g. as disclosed in EP-A1-0 505 322, Br. J. Pharmacol., 115,1387, 1995 or in the guinea pig distal colon test e.g. as disclosed in Br. J. Pharm., 1593-1599, 1993). Exemplary 5-HT4-partial-agonists include (1-(4-amino-5-chloro-2-methoxyphenyl)-3-[1-butyl-4-piperidinyl]-1-propanone or (1-(4-amino-5-chloro-2-methoxyphenyl)-3-[1-(methylsulphonylamino)ethyl-4-piperidinyl]-1-propanone or, in particular, those compounds disclosed in EP0505322, e.g. TEGASEROD.

A second subaspect of the the expression “5-HT-(partial-)agonists/antagonists” according to said special aspect g refers to 5-HT4-agonists. In this context, 5-HT4-agonists include any compound which can activate 5-HT4-receptors under quiescent/resting conditions, such as, for example, CISAPRIDE, NOR-CISAPRIDE, ZACOPRIDE, SB 205149, SC 53116, SL-65.0155, E-3620, RS 67333, RS 67506, BIMU-1, BIMU-8 or (S)-RS 56532, or, in particular, MOSAPRIDE or PRUCALOPRIDE.

A third subaspect of the the expression “5-HT-(partial-)agonists/antagonists” according to said special aspect g refers to 5-HT3-antagonists. In this context, 5-HT3 receptor antagonists include any compound which binds to the 5-HT3 receptor and antagonize the effect of 5-HT3-agonists, such as, for example, in one facet, CILANSETRON, ALOSETRON, RAMOSETRON, AZASETRON, ONDANSETRON, DOLASETRON, GRANISETRON, or TROPISETRON;

or, in another facet, BENESETRON, ZATOSETRON, EM-523, ZACOPRIDE, DAZOPRIDE, BATANOPRIDE, AS-5370, MCL-225, WAY-100289, YM-114, CILANSETRON, LERISETRON, MIRESETRON, RS-25259-197, T-82, INDISETRON, or RS42358-197, or in particular DOLASETRON, PALONOSETRON, AZASETRON, TROPISETRON, ONDANSETRON, GRANISETRON, ALOSETRON, RAMOSETRON or INDISETRON.

A fourth subaspect of the the expression “5-HT-(partial-)agonists/antagonists” according to said special aspect g refers to compounds which activates and/or binds to 5-HT receptors and which are not either 5-HT4-partial-agonists or 5-HT4-antagonists as defined herein. Exemplary compounds according to this fourth subaspect are those 5-HT-(partial-)agonists/antagonists, which are mentioned expressis verbis in this description, with the provisio that 5-HT4-partial-agonists and 5-HT4-antagonists are thereof disclaimed.

A fifth subaspect of the the expression “5-HT-(partial-)agonists/antagonists” according to said special aspect g refers to any compound which binds to the 5-HT4 receptor as defined by the IUPHAR (Pharmacological Reviews, Vol. 44, p. 157-213, 1994) and that do not activate the 5-HT4 receptor and antagonize the effects of serotonin. A relevant test to determine whether or not a compound is a 5-HT4-antagonist is the Guinea-Pig distal colon test as described in Br. J. Pharm., p. 1593-1599 (1993) or in the test described in Arch. Pharmacol., Vol. 343, p. 439-446 (1991). Representative 5-HT4 antagonists include e.g. PIBOSEROD; A-85380 (WO 9408994); SB 204070 (Drugs Fut., 19: 1109-1121, 1994); SB 207058 (Exp. Opin. Invest. Drugs, 3 (7): 767, 1994); SB 207710 (Drug Data Report, 15 (10): 949, 1993); SB 205800 (Drug Data Report, 15 (10): 949,1993); SB 203186 (Br. J. Pharmacol., 110: 10231030, 1993); N 3389 (Eur. J. Pharmacol., 271: 159, 1994); FK 1052 (J. Pharmacol. Exp. Ther., 265: 752, 1993); SC 56184 (R & D Focus, 2 (37) 10,1993); SC 53606 (J. Pharmacol. Exp. Ther. 226: 1339, 1993); DAU 6285 (Br. J. Pharmacol., 105: 973, 1992); GR 125487 (Br. J. Pharmacol., 113 suppl. 119P & 120P, 1994); GR 113808 (Br. J. Pharmacol. 110: 1172,1993); RS 23597 (Bioorg Med. Chem. Lett., 4 (20): 2477, 1994); RS 39604 (Br. J. Pharmacol., 115, 1087-1095, 1995); LY-353433 (EP 0732333, J. Pharmacol. Exp. Ther, 277 (1), 97-104, 1996); and R59595 (Eur. J. Pharmacol., 212, 51-59,1992); whereby PIBOSEROD (WO 9318036) and LY-353433 (EP 0732333) are particularly emphasized.

A sixth subaspect of the the expression “5-HT-(partial-)agonists/antagonists” according to said special aspect g refers to dual 5-HT3/5-HT4-agonists/antagonists, i.e. e.g. compounds which show characteristics of 5-HT3 receptor antagonists and 5-HT4 receptor agonists or antagonists such as, for example, CISAPRIDE and NOR-CISAPRIDE; BIMU compounds, for example BIMU1, BIMU8 and DAU 6215 (also known as ITASETRON) as disclosed in Dumuis A., et al., Naunyn Schmiedebers Arch. Pharmacol., Vol. 343 (3), pp. 245-251 (1991); DAU-6236 as disclosed in Rizzi, C. A. et al., J. Pharmacol. Exp. Ther., Vol. 261, pp. 412-419 (1992); and DAU-6258, Turconi M, et al., J. Med. Chem., Vol. 33 (8), pg. 2101-2108 (1990), SDZ 205557 which is a benzoic acid derivative (ester) Eglen R. M. et al., Proc. Br. Pharmacol. Soc., Vol. 149 (1992); RENZAPRIDE; ZACOPRIDE; SB 205149; SC 53116; RS 67333; RS 67506; or (S)-RS 56532, LINTOPRIDE; or FABESETRON or E-3620.

A seventh subaspect of the the expression “5-HT-(partial-)agonists/antagonists” according to said special aspect g refers to compounds, which activates or binds to 5-HT receptors, and which are not 5-HT4-partial-agonists. Exemplary compounds according to this seventh subaspect are those 5-HT-(partial-)agonists/antagonists, which are mentioned expressis verbis herein, with the provisio that 5-HT4-partial-agonists are thereof disclaimed.

An eigth subaspect of the the expression “5-HT-(partial-)agonists/antagonists” according to said special aspect g refers to compounds, which activates or binds to 5-HT receptors, and which are not 5-HT4-antagonists as defined herein. Exemplary compounds according to this eighth subaspect are those 5-HT-(partial-)agonists/antagonists, which are mentioned expressis verbis herein, with the provisio that 5-HT4-antagonists are thereof disclaimed.

A nineth subaspect of the the expression “5-HT-(partial-)agonists/antagonists” according to said special aspect g refers to compounds, which activates or binds to 5-HT receptors, and which are not either selective 5-HT4-partial-agonists or selective 5-HT4-anatgonists. Exemplary compounds according to this nineth subaspect are those 5-HT-(partial-)agonists/antagonists, which are mentioned expressis verbis herein, with the provisio that selective 5-HT4-partial-agonists and selective 5-HT4-antagonists are thereof disclaimed. The term “selective” means in this context a compound which does not substantially bind to or stimulate the 5-HT3 receptor subtype.

A tenth subaspect of the the expression “5-HT-(partial-)agonists/antagonists” according to said special aspect g refers to compounds, which activates or binds to 5-HT receptors, and which act not both on 5-HT3 and 5-HT4 receptor. Exemplary compounds according to this tenth subaspect are those 5-HT-(partial-)agonists/antagonists, which are mentioned expressis verbis herein, with the provisio that dual 5-HT4/5-HT3 agonists/antagonists are thereof disclaimed.

An eleventh subaspect of the the expression “5-HT-(partial-)agonists/antagonists” according to said special aspect g refers to compounds, which activates or binds to 5-HT receptors, and which are not selective 5-HT4-partial-agonists, selective 5-HT3-anatgonists or dual 5-HT3/5-HT4-agonists/antagonists. Exemplary compounds according to this eleventh subaspect are those 5-HT-(partial-)agonists/antagonists, which are mentioned expressis verbis herein, with the provisio that selective 5-HT4-partial-agonists, selective 5-HT4-antagonists and dual 5-HT4/5-HT3 agonists/antagonists are thereof disclaimed.

A twelfth subaspect of the the expression “5-HT-(partial-)agonists/antagonists” according to said special aspect g refers to 5-HT3-agonists, such as, for example, YM-31636, or, particularly, PUMOSETRAG.

An eighth special aspect (aspect h) of the term “compounds, which modify gastrointestinal motility” refers to those compounds, which are selected from

the class of GABA-A and, in particular, of the class of GABA-B receptor agonists/partial agonists.

As exemplary compounds according to said special aspect h are to be mentioned, without being restricted thereto, those compounds mentioned or specified in the description of this invention.

A ninth special aspect (aspect i) of the term “compounds, which modify gastrointestinal motility” refers to those compounds, which are selected from a group consisting of muscarinic antagonists, kappa opioid receptor agonists, delta opioid receptor agonists, opioid receptor agonists, dopamine receptor antagonists, cholecystokinin A antagonists, alpha-2 adrenoceptor agonists, N-methyl-D-aspartate receptor antagonists, non-N-methyl-D-aspartate glutamate receptor antagonists, nitric oxide synthase inhibitors, motilin agonists, somatostatin agonists/antagonists, neurotensin agonists/antagonists, vasoactive intestinal peptide antagonists, substance P antagonists, neurokinin antagonists, calcium channel blockers, potassium channel openers, selective serotonin reuptake inhibitors, corticotropin releasing factor antagonists, GABA-A receptor agonists, GABA-B receptor agonists/partial agonists, gastroprokinetics, antiemetics and antispasmodics, and which are not 5-HT-(partial-)agonists/antagonists.

A tenth special aspect (aspect j) of the term “compounds, which modify gastrointestinal motility” refers to those compounds, which are mentioned or specified expressis verbis or by reference in the description of this invention, and which are not 5-HT-(partial-)agonists/antagonists.

An eleventh special aspect (aspect k) of the term “compounds, which modify gastrointestinal motility” refers to those compounds, which are mentioned or specified expressis verbis or by reference in the description of this invention, and which are not 5-HT4-partial-agonists or 5-HT4-antagonists.

A twelfth special aspect (aspect l) of the term “compounds, which modify gastrointestinal motility” refers to those compounds, which are mentioned or specified expressis verbis or by reference in the description of this invention, whereby

5-HT4-partial-agonists,

5-HT4-antagonists, and

dual 5-HT3 antagonists/5-HT4 agonists

are thereof disclaimed.

A thirteenth special aspect (aspect m) of the term “compounds, which modify gastrointestinal motility” refers to those compounds, which are mentioned or specified expressis verbis or by reference in the description of this invention, and which show characteristics of 5-HT3-antagonists and 5-HT4-agonists or antagonists.

A fourteenth special aspect (aspect n) of the term “compounds, which modify gastrointestinal motility” refers to those compounds, which are mentioned or specified expressis verbis or by reference in the description of this invention, and which are selective 5-HT3-antagonists (this means non-dual 5-HT3-antagonists i.e. 5-HT3-antagonists not being 5-HT4-agonists).

A fifteenth special aspect (aspect o) of the term “compounds, which modify gastrointestinal motility” refers to those compounds, which are mentioned or specified expressis verbis or by reference in the description of this invention, and which are 5-HT3-agonists.

A sixteenth special aspect (aspect p) of the term “compounds, which modify gastrointestinal motility” refers to those compounds, which are mentioned or specified expressis verbis or by reference in the description of this invention, and which are selective 5-HT4-agonists (this means non-dual 5-HT4-agonists i.e. 5-HT4-agonists not being 5-HT3-antagonists).

A seventeenth special aspect (aspect q) of the term “compounds, which modify gastrointestinal motility” refers to those compounds, which are mentioned or specified expressis verbis or by reference in the description of this invention, and which are 5-HT4-partial-agonists.

An eighteenth special aspect (aspect r) of the term “compounds, which modify gastrointestinal motility” refers to those compounds, which are mentioned or specified expressis verbis or by reference in the description of this invention, and which are 5-HT4-antagonists.

A nineteenth special aspect (aspect s) of the term “compounds, which modify gastrointestinal motility” refers to those compounds, which are mentioned or specified expressis verbis or by reference in the description of this invention, and which are dual 5-HT3 antagonists/5-HT4 agonists.

In the meaning of the present invention, two or more of the special aspects a to s according to this invention can be combined to give special subaspects thereof; or two or more of the special aspects a to s can be combined to give further special aspects of the term “compounds, which modify gastrointestinal motility” according to this invention.

In one facet of this invention, special aspects of the term “compounds, which modify gastrointestinal motility” to be more worthy to be mentioned in the meaning of this invention are aspect a, aspect b, aspect c, aspect g and aspect h.

In a further facet of this invention, special aspects of the term “compounds, which modify gastrointestinal motility” to be further more worthy to be mentioned in the meaning of this invention are aspect a, aspect b, aspect c, aspect e, aspect g and aspect h.

In a particular facet of this invention, a special aspect of the term “compounds, which modify gastrointestinal motility” to be mentioned as interesting within the meaning of this invention is aspect g.

Yet in a particular facet of this invention (particularly regarding the inhibition of transient lower esophageal sphincter relaxations), a special aspect of the term “compounds, which modify gastrointestinal motility” to be mentioned as interesting within the meaning of this invention is aspect h.

In a further particular facet of this invention (particularly regarding the therapy of GERD), special aspects of the term “compounds, which modify gastrointestinal motility” to be mentioned as particular interesting within the meaning of this invention are aspect a and/or aspect c.

Yet in a further particular facet of this invention (particularly regarding the therapy of IBS), a special aspect of the term “compounds, which modify gastrointestinal motility” to be mentioned as particular interesting within the meaning of this invention is aspect b.

In yet another particular facet of this invention, a special aspect of the term “compounds, which modify gastrointestinal motility” to be mentioned as particular interesting within the meaning of this invention is aspect e.

In the context of said aspect g more worthy to be mentioned, a first subaspect of the present invention relates to a pharmaceutical composition comprising a first agent which is a 5-HT-(partial-)agonist/antagonist such as, for example, one of those mentioned above; and a second agent which is an acid pump antagonist selected from a group consisting of those acid pump antagonists mentioned or accentuated above expressis verbis or by reference with the provisio that Pumaprazole, SKF 97574, SKF 96067, H 40502, YH1238 and YH1885 are thereof disclaimed.

In further context of said aspect g more worthy to be mentioned, a second subaspect of the present invention relates to a pharmaceutical composition comprising a first agent which is a 5-HT-(partial-)ago-nist/antagonist such as, for example, one of those disclosed generically or, in particular, specifically in the international application WO 0141748 as useful to be employed in combination with coagents; and a second agent which is an acid pump antagonist selected from a group consisting of those acid pump antagonists mentioned or accentuated above expressis verbis or by reference with the provisio that Pumaprazole, SKF 97574, SKF 96067, H 40502, YH1238 and YH1885 are thereof disclaimed.

In still further context of said aspect g more worthy to be mentioned, a third subaspect of the present invention relates to a pharmaceutical composition comprising a first agent which is a 5-HT-(partial-)ago-nist/antagonist such as, for example, 3-(5-methoxy-1H-indol-3-yl-methylene)-N-pentylcarbazimidamide, which is also known as tegaserod, or a salt (e.g. the hydrogen maleate) or a tautomere thereof; and a second agent which is an acid pump antagonist selected from a group consisting of (7R,8R,9R)-2.3-dimethyl-8-hydroxy-7-(2-methoxyethoxy)-9-phenyl-7.8.9.10-tetrahydroimidazo-[1.2-h][1.7]naphthyridin,
(7R,8R,9R)-2,3-dimethyl-8-hydroxy-7-(2-methoxyethoxy)-g-phenyl-7H-8,9-dihydro-pyrano[2,3-c]-imidazo[1,2-a]pyridine and
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
   and of the salts, solvates and solvates of the salts of these compounds.

In yet further context of said aspect g more worthy to be mentioned, a fourth subaspect of the present invention relates to a pharmaceutical composition or combination comprising a first agent which is a 5-HT-(partial-)agonist/antagonist such as, for example, one of those disclosed generically or, in particular, specifically in the international application US20040092511 as useful to be employed in combination with co-agents; and a second agent which is an acid pump antagonist selected from a group consisting of those acid pump antagonists mentioned or accentuated above expressis verbis or by reference with the provisio that Pumaprazole, SKF 97574, SKF 96067, H 40502, BY 112, YH1238 and YH1885 are thereof disclaimed.

In still yet further context of said aspect g more worthy to be mentioned, a fifth subaspect of the present invention relates to a pharmaceutical composition or combination comprising a first agent which is a mixed i.e. dual 5-HT3-antagonist/5-HT4 agonist such as e.g. CISAPRIDE or NOR-CISAPRIDE, i.e. (±)-NOR-CISAPRIDE, (−)-NOR-CISAPRIDE, or, particularly, (+)-NOR-CISAPRIDE, or TICALOPRIDE; and a second agent which is an acid pump antagonist selected from a List A, or in particular List C, or in more particular Soraprazan.

A particular embodiment according to the present invention refers to a combination comprising

a first active ingredient which is any acid pump antagonist according to detail a, in particular an acid pump antagonist selected from List A, in more particular selected from List C; and

a second active ingredient which is a compound, which modifies gastrointestinal motility, in particular, any compound or class of compounds according to special aspect a and/or h; for simultaneous, sequential, separate or chronologically staggered use in therapy in any order.

Another particular embodiment according to the present invention refers to a combination comprising

a first active ingredient which is any acid pump antagonist according to detail a, in particular an acid pump antagonist selected from List A, In more particular selected from List C; and

a second active ingredient which is a compound, which modifies gastrointestinal motility, in particular, any compound or class of compounds according to special aspect b;

for simultaneous, sequential, separate or chronologically staggered use in therapy in any order.

Another particular embodiment according to the present invention refers to a combination comprising

a first active ingredient which is any acid pump antagonist according to detail a, in particular an acid pump antagonist selected from List A, in more particular selected from List C; and

a second active ingredient which is a compound, which modifies gastrointestinal motility, in particular, any compound or class of compounds according to special aspect c;

for simultaneous, sequential, separate or chronologically staggered use in therapy in any order.

Another particular embodiment according to the present invention refers to a combination comprising

a first active ingredient which is any acid pump antagonist according to detail a, in particular an acid pump antagonist selected from List A, in more particular selected from List C; and

a second active ingredient which is a compound, which modifies gastrointestinal motility, in particular, any compound or class of compounds according to special aspect e;

for simultaneous, sequential, separate or chronologically staggered use in therapy in any order.

Another particular embodiment according to the present invention refers to a combination comprising

a first active ingredient which is any acid pump antagonist according to detail a, in particular an acid pump antagonist selected from List A, in more particular selected from List C; and

a second active ingredient which is a compound, which modifies gastrointestinal motility, in particular, any compound or class of compounds according to special aspect g;

for simultaneous, sequential, separate or chronologically staggered use in therapy in any order.

Yet another particular embodiment according to the present invention refers to a combination comprising

a first active ingredient which is any acid pump antagonist according to detail b, in particular an acid pump antagonist selected from List B; and

a second active ingredient which is a compound, which modifies gastrointestinal motility, in particular, any compound or class of compounds according to special aspect a and/or h;

for simultaneous, sequential, separate or chronologically staggered use in therapy in any order.

Yet another particular embodiment according to the present invention refers to a combination comprising

a first active ingredient which is any acid pump antagonist according to detail b, in particular an acid pump antagonist selected from List B; and

a second active ingredient which is a compound, which modifies gastrointestinal motility, in particular, any compound or class of compounds according to special aspect b;

for simultaneous, sequential, separate or chronologically staggered use in therapy in any order.

Yet another particular embodiment according to the present invention refers to a combination comprising

a first active ingredient which is any acid pump antagonist according to detail b, in particular an acid pump antagonist selected from List B; and

a second active ingredient which is a compound, which modifies gastrointestinal motility, in particular, any compound or class of compounds according to special aspect c;

for simultaneous, sequential, separate or chronologically staggered use in therapy in any order.

Yet another particular embodiment according to the present invention refers to a combination comprising

a first active ingredient which is any acid pump antagonist according to detail b, in particular an acid pump antagonist selected from List B; and

a second active ingredient which is a compound, which modifies gastrointestinal motility, in particular, any compound or class of compounds according to special aspect e;

for simultaneous, sequential, separate or chronologically staggered use in therapy in any order.

Yet another particular embodiment according to the present invention refers to a combination comprising

a first active ingredient which is any acid pump antagonist according to detail b, in particular an acid pump antagonist selected from List B; and

a second active ingredient which is a compound, which modifies gastrointestinal motility, in particular, any compound or class of compounds according to special aspect g;

for simultaneous, sequential, separate or chronologically staggered use in therapy in any order.

Yet another particular embodiment according to the present invention refers to a combination comprising

a first active ingredient which is any acid pump antagonist according to detail b, in particular an acid pump antagonist selected from List B; and

a second active ingredient which is

any 5-HT4-partial-agonist such as e.g. TEGASEROD;

any 5-HT4-agonist such as e.g. MOSAPRIDE or PRUCALOPRIDE;

any 5-HT3 receptor antagonist such as e.g. CILANSETRON, ALOSETRON, RAMOSETRON, AZASETRON, ONDANSETRON, DOLASETRON, GRANISETRON, or TROPISETRON;

any 5-HT4 antagonist such as e.g. PIBOSEROD, or LY-353433;

any dual 5-HT3-antagonist/5-HT4-agonist such as e.g. CISAPRIDE, NOR-CISAPRIDE, (+)-NORCISAPRIDE, BIMU1, BIMU8, RENZAPRIDE, ZACOPRIDE, LINTOPRIDE, ITASETRON, FABESETRON, or E-3620;

for simultaneous, sequential, separate or chronologically staggered use in therapy in any order.

Yet another particular embodiment according to the present invention refers to a combination comprising

a first active ingredient which is any acid pump antagonist according to detail b, in particular an acid pump antagonist selected from List B; and

a second active ingredient which is

PRUCALOPRIDE or CILANSETRON, or, in particular,

ALOSETRON, or, in more particular,

TEGASEROD,

for simultaneous, sequential, separate or chronologically staggered use in therapy in any order.

Yet another particular embodiment according to the present invention refers to a combination comprising

a first active ingredient which is any add pump antagonist according to detail b, in particular an acid pump antagonist selected from List B; and

a second active ingredient which is selected from the group consisting of

TEGASEROD, ALOSETRON, CILANSETRON, PRUCALOPRIDE, ALVIMOPAN, PIBOSEROD and DEXLOXIGLUMIDE;

for simultaneous, sequential, separate or chronologically staggered use in therapy in any order.

Yet another particular embodiment according to the present invention refers to a combination comprising

a first active ingredient which is any acid pump antagonist according to detail b, in particular an acid pump antagonist selected from List B; and

a second active ingredient which is TEGASEROD, or a salt or a tautomer thereof, such as e.g. TEGASEROD MESYLATE (Zelmac) or MALEATE (Zelnorm);

for simultaneous, sequential, separate or chronologically staggered use in therapy in any order.

Still yet another particular embodiment according to the present invention refers to a combination comprising

a first active ingredient which is any acid pump antagonist according to detail c, in particular an acid pump antagonist selected from group x according to detail c; and

a second active ingredient which is a compound, which modifies gastrointestinal motility, in particular, any compound or class of compounds according to special aspect b;

for simultaneous, sequential, separate or chronologically staggered use in therapy in any order.

Still yet another particular embodiment according to the present invention refers to a combination comprising

a first active ingredient which is any acid pump antagonist according to detail c, in particular an acid pump antagonist selected from group x according to detail c; and

a second active ingredient which is a compound, which modifies gastrointestinal motility, in particular, any compound or class of compounds according to special aspect c, whereby compounds which reduce the incidence of transient lower esophageal sphincter relaxation (TLOSR), e.g. GABA-B agonists, are thereof disclaimed;

for simultaneous, sequential, separate or chronologically staggered use in therapy in any order.

Still yet another particular embodiment according to the present invention refers to a combination comprising

a first active ingredient which is any acid pump antagonist according to detail c, in particular an acid pump antagonist selected from group x according to detail c; and

a second active ingredient which is a compound, which modifies gastrointestinal motility, in particular, any compound or class of compounds according to special aspect e, whereby compounds which reduce the incidence of transient lower esophageal sphincter relaxation (TLOSR), e.g. GABA-B agonists, are thereof disclaimed;

for simultaneous, sequential, separate or chronologically staggered use in therapy in any order.

Still yet another particular embodiment according to the present invention refers to a combination comprising

a first active ingredient which is any acid pump antagonist according to detail c, in particular an acid pump antagonist selected from group x according to detail c; and

a second active ingredient which is a compound, which modifies gastrointestinal motility, in particular, any compound or class of compounds according to special aspect g;

for simultaneous, sequential, separate or chronologically staggered use in therapy in any order.

Still yet another particular embodiment according to the present invention refers to a combination comprising

a first active ingredient which is any acid pump antagonist according to detail c, in particular an acid pump antagonist selected from group x according to detail c; and

a second active ingredient which is any 5-HT4-partial-agonist such as e.g. TEGASEROD;

any 5-HT4-agonist such as e.g. MOSAPRIDE or PRUCALOPRIDE;

any 5-HT3 receptor antagonist such as e.g. CILANSETRON, ALOSETRON, RAMOSETRON, AZASETRON, ONDANSETRON, DOLASETRON, GRANISETRON, or TROPISETRON;

any 5-HT4 antagonist such as e.g. PIBOSEROD, or LY-353433;

any dual 5-HT3-antagonist/5-HT4-agonist such as e.g. CISAPRIDE, NOR-CISAPRIDE, (+)-NORCISAPRIDE, BIMU1, BIMU8, RENZAPRIDE, ZACOPRIDE, LINTOPRIDE, ITASETRON, FABESETRON, or E-3620;

for simultaneous, sequential, separate or chronologically staggered use in therapy in any order.

Still yet another particular embodiment according to the present invention refers to a combination comprising

a first active ingredient which is any acid pump antagonist according to detail c, in particular an acid pump antagonist selected from group x according to detail c; and

a second active ingredient which is

PRUCALOPRIDE or CILANSETRON, or, in particular,

ALOSETRON, or, in more particular,

TEGASEROD,

for simultaneous, sequential, separate or chronologically staggered use in therapy in any order.

Still yet another particular embodiment according to the present invention refers to a combination comprising

a first active ingredient which is any acid pump antagonist according to detail c, in particular an acid pump antagonist selected from group x according to detail c; and

a second active ingredient which is selected from the group consisting of

TEGASEROD, ALOSETRON, CILANSETRON, PRUCALOPRIDE, ALVIMOPAN, PIBOSEROD and DEXLOXIGLUMIDE;

for simultaneous, sequential, separate or chronologically staggered use in therapy in any order.

Still yet another particular embodiment according to the present invention refers to a combination comprising

a first active ingredient which is any acid pump antagonist according to detail c, in particular an acid pump antagonist selected from group x according to detail c; and

a second active ingredient which is TEGASEROD, or a salt or a tautomer thereof, such as e.g. TEGASEROD MESYLATE (Zelmac) or MALEATE (Zelnorm);

for simultaneous, sequential, separate or chronologically staggered use in therapy in any order.

A further particular embodiment according to the present invention refers to a combination comprising

a first active ingredient which is (7R,8R,9R)-8-hydroxy-7-(2-methoxyethoxy)-2,3-dimethyl-9-phenyl-7,8,9,10-tetrahydro-imidazo[1,2-h][1,7]naphthyridine, or a salt, solvate or solvate of the salt thereof; and

a second active ingredient which is a compound, which modifies gastrointestinal motility, in particular, any compound or class of compounds according to special aspect a and/or h;

for simultaneous, sequential, separate or chronologically staggered use in therapy in any order.

A further particular embodiment according to the present invention refers to a combination comprising

a first active ingredient which is (7R,8R,9R)-8-hydroxy-7-(2-methoxyethoxy)-2,3-dimethyl-9-phenyl-7,8,9,10-tetrahydro-imidazo[1,2-h][1,7]naphthyridine, or a salt, solvate or solvate of the salt thereof; and

a second active ingredient which is a compound, which modifies gastrointestinal motility, in particular, any compound or class of compounds according to special aspect b;

for simultaneous, sequential, separate or chronologically staggered use in therapy in any order.

A further particular embodiment according to the present invention refers to a combination comprising

a first active ingredient which is (7R,8R,9R)-8-hydroxy-7-(2-methoxyethoxy)-2,3-dimethyl-9-phenyl-7,8,9,10-tetrahydro-imidazo[1,2-h][1,7]naphthyridine, or a salt, solvate or solvate of the salt thereof; and

a second active ingredient which is a compound, which modifies gastrointestinal motility, in particular, any compound or class of compounds according to special aspect c;

for simultaneous, sequential, separate or chronologically staggered use in therapy in any order.

A further particular embodiment according to the present invention refers to a combination comprising

a first active ingredient which is (7R,8R,9R)-8-hydroxy-7-(2-methoxyethoxy)-2,3-dimethyl-9-phenyl-7,8,9,10-tetrahydro-imidazo[1,2-h][1,7]naphthyridine, or a salt, solvate or solvate of the salt thereof; and

a second active ingredient which is a compound, which modifies gastrointestinal motility, in particular, any compound or class of compounds according to special aspect e;

for simultaneous, sequential, separate or chronologically staggered use in therapy in any order.

A further particular embodiment according to the present invention refers to a combination comprising

a first active ingredient which is (7R,8R,9R)-8-hydroxy-7-(2-methoxyethoxy)-2,3-dimethyl-9-phenyl-7,8,9,10-tetrahydro-imidazo[1,2-h][1,7]naphthyridine, or a salt, solvate or solvate of the salt thereof; and

a second active ingredient which is a compound, which modifies gastrointestinal motility, in particular, any compound or class of compounds according to special aspect g;

for simultaneous, sequential, separate or chronologically staggered use in therapy in any order.

A particular embodiment according to the present invention (embodiment a1) to be emphasized refers to a combination comprising

a first active ingredient which is any acid pump antagonist according to detail a, in particular an acid pump antagonist selected from List A; and

a second active ingredient which is a compound, which modifies gastrointestinal motility, in particular, in one independent embodimental variant, any compound or class of compounds mentioned in special aspect a, or

in another independent embodimental variant, any compound or class of compounds mentioned in special aspect b, or

in another independent embodimental variant, any compounds mentioned specifically or generically in special aspect c, or

in another independent embodimental variant, from the compounds mentioned specifically or generically in special aspect e, or

in another independent embodimental variant, from the compounds mentioned specifically or generically in special aspect g, or

in another independent embodimental variant, from the compounds mentioned specifically or generically in special aspect h;

for simultaneous, sequential, separate or chronologically staggered use in therapy in any order, e.g. to treat GERD or IBS.

A particular embodiment according to the present invention (embodiment a2) to be more emphasized refers to a combination comprising

a first active ingredient which an acid pump antagonist selected from List C; and

a second active ingredient which is a compound, which modifies gastrointestinal motility, in particular, in one independent embodimental variant, any compound or class of compounds mentioned in special aspect a, or

in another independent embodimental variant, any compound or class of compounds mentioned in special aspect b, or

in another independent embodimental variant, any compounds mentioned specifically or generically in special aspect c, or

in another independent embodimental variant, from the compounds mentioned specifically or generically in special aspect e, or

in another independent embodimental variant, from the compounds mentioned specifically or generically in special aspect g, or

in another independent embodimental variant, from the compounds mentioned specifically or generically in special aspect h;

for simultaneous, sequential, separate or chronologically staggered use in therapy in any order, e.g. to treat GERD or IBS.

A particular embodiment according to the present invention (embodiment a3) to be in particular emphasized refers to a combination comprising

a first active ingredient which is (7R,8R,9R)-8-hydroxy-7-(2-methoxyethoxy)-2,3-dimethyl-9-phenyl-7,8,9,10-tetrahydro-imidazo[1,2-h][1,7]naphthyridine, or a salt, solvate or solvate of the salt thereof; and

a second active ingredient which is a compound, which modifies gastrointestinal motility, in particular, in one independent embodimental variant, any compound or class of compounds mentioned in special aspect a, or

in another independent embodimental variant, any compound or class of compounds mentioned in special aspect b, or

in another independent embodimental variant, any compounds mentioned specifically or generically in special aspect c, or

in another independent embodimental variant, from the compounds mentioned specifically or generically in special aspect e, or

in another independent embodimental variant, from the compounds mentioned specifically or generically in special aspect g, or

in another independent embodimental variant, from the compounds mentioned specifically or generically in special aspect h;

for simultaneous, sequential, separate or chronologically staggered use in therapy in any order, e.g. to treat GERD or IBS.

Another particular embodiment according to the present invention (embodiment a4) to be emphasized refers to a combination comprising

a first active ingredient which is any acid pump antagonist according to detail a, in particular an acid pump antagonist selected from List A, or, in more particular, selected from List C; and

a second active ingredient which is a compound, which reduce the incidence of transient lower esophageal sphincter relaxation (TLOSR), such as e.g. a GABA-B receptor agonist, in particular a GABA-B receptor agonist selected,

in one independent embodimental variant, from list 23a, or

in another independent embodimental variant, from the list consisting of

4-amino-3-(4-chlorophenyl)butanoic acid (baclofen), (3-aminopropyl)methylphosphinic acid, (3-amino-2-hydroxypropyl)methylphosphinic acid, (3-amino-2-(4-chlorophenyl)propyl)sulfinic acid, (3-aminopropyl)(difluoromethyl)phosphinic acid, (3-amino-2-oxo-propyl)methyl phosphinic acid and 4 amino-35-chlorothien-2-yl)butanoic acid and (3-aminopropyl)phosphonous acid, or, in particular,

in yet another independent embodimental variant, from the list 23b;

for simultaneous, sequential, separate or chronologically staggered use in therapy in any order, e.g. to treat GERD.

Another particular embodiment according to the present invention (embodiment a5) to be in particular emphasized refers to a combination comprising

a first active ingredient which is (7R,8R,9R)-8-hydroxy-7-(2-methoxyethoxy)-2,3-dimethyl-9-phenyl-7,8,9,10-tetrahydro-imidazo[1,2-h][1,7]naphthyridine, or a salt, solvate or solvate of the salt thereof; and

a second active ingredient which is a compound, which reduce the incidence of transient lower esophageal sphincter relaxation (TLOSR), such as e.g. a GABA-B receptor agonist, in particular a GABA-B receptor agonist selected,

in one independent embodimental variant, from list 23a, or in another independent embodimental variant, from the list consisting of

4-amino-3-(4-chlorophenyl)butanoic acid (baclofen), (3-aminopropyl)methylphosphinic acid, (3-amino-2-hydroxypropyl)methylphosphinic acid, (3-amino-2-(4-chlorophenyl)propyl)sulfinic acid, (3-aminopropyl)(difluoromethyl)phosphinic acid, (3-amino-2-oxo-propyl)methyl phosphinic acid and 4 amino-3-(5-chlorothien-2-yl)butanoic acid and (3-aminopropyl)phosphonous acid, or, in particular,

in yet another independent embodimental variant, from the list 23b;

for simultaneous, sequential, separate or chronologically staggered use in therapy in any order, e.g. to treat GERD.

Yet another particular embodiment according to the present invention (embodiment a6) to be emphasized refers to a combination comprising

a first active ingredient which is any acid pump antagonist according to detail a, in particular an acid pump antagonist selected from List A, in more particular selected from List C; and

a second active ingredient which is

any 5-HT4-partial-agonist such as e.g. TEGASEROD;

any 5-HT4-agonist such as e.g. MOSAPRIDE or PRUCALOPRIDE;

any 5-HT3 receptor antagonist such as e.g. CILANSETRON, ALOSETRON, RAMOSETRON, AZASETRON, ONDANSETRON, DOLASETRON, GRANISETRON, or TROPISETRON;

any 5-HT4 antagonist such as e.g. PIBOSEROD, or LY-353433;

any dual 5-HT3-antagonist/5-HT4-agonist such as e.g. CISAPRIDE, NOR-CISAPRIDE, (+)-NORCISAPRIDE, BIMU1, BIMU8, RENZAPRIDE, ZACOPRIDE, LINTOPRIDE, ITASETRON, FABESETRON, or E-3620;

for simultaneous, sequential, separate or chronologically staggered use in therapy in any order.

Yet another particular embodiment according to the present invention (embodiment a7) to be emphasized refers to a combination comprising

a first active ingredient which is any acid pump antagonist according to detail a, in particular an acid pump antagonist selected from List A; in more particular selected from List C; and

a second active ingredient which is

PRUCALOPRIDE or CILANSETRON, or, in particular,

ALOSETRON, or, in more particular,

TEGASEROD,

for simultaneous, sequential, separate or chronologically staggered use in therapy in any order.

Yet another particular embodiment according to the present invention (embodiment a8) to be emphasized refers to a combination comprising

a first active ingredient which is any acid pump antagonist according to detail a, in particular an acid pump antagonist selected from List A; in more particular selected from List C; and

a second active ingredient which is selected from the group consisting of

TEGASEROD, ALOSETRON, CILANSETRON, PRUCALOPRIDE, ALVIMOPAN, PIBOSEROD and DEXLOXIGLUMIDE;

for simultaneous, sequential, separate or chronologically staggered use in therapy in any order.

Yet another particular embodiment according to the present invention (embodiment a9) to be emphasized refers to a combination comprising

a first active ingredient which is any acid pump antagonist according to detail a, in particular an acid pump antagonist selected from List A; in more particular selected from List C; and

a second active ingredient which is TEGASEROD, or a salt or a tautomer thereof, such as e.g.

TEGASEROD MESYLATE (Zelmac) or MALEATE (Zelnorm);

for simultaneous, sequential, separate or chronologically staggered use in therapy in any order.

Yet another particular embodiment according to the present invention (embodiment a10) to be more emphasized refers to a combination comprising

a first active ingredient which is any acid pump antagonist selected from List C; and

a second active ingredient which refers to any compound or class of compounds of TICALOPRIDE,

5-HT4 antagonists, such as e.g. PIBOSEROD, or LY-353433,

5-HT3 antagonists, such as e.g. YM-114, or CILANSETRON, RAMOSETRON or ALOSETRON,

5-HT4 partial agonists, such as e.g. TEGASEROD,

5-HT4 agonists, such as e.g. PRUCALOPRIDE,

dual 5-HT3 antagonists/5-HT4 agonists, such as e.g. FABESETRON, or E-3620 or RENZAPRIDE;

cholecystokinin A antagonists, such as e.g. DEXLOXIGLUMIDE;

NK-2 antagonists, such as e.g. NEPADUTANT or SAREDUTANT,

NK-3 antagonists, such as e.g. TALNETANT;

kappa oploid receptor agonists, such as e.g. FEDOTOZINE, PTI-901 or ASIMADOLINE;

delta opioid receptor agonists, such as e.g. ALVIMOPAN; or

muscarinic M3 antagonists, such as e.g. ZAMIFENACIN, or (SYOXYBUTININ, J-104135 or DARIFENAZIN;

for simultaneous, sequential, separate or chronologically staggered use in therapy in any order, e.g. to treat or prevent IBS.

Yet another particular embodiment according to the present invention (embodiment a11) to be more emphasized refers to a combination comprising

a first active ingredient which is any acid pump antagonist selected from List C; and

a second active ingredient which refers to any compound or class of compounds of TICALOPRIDE,

5-HT4 partial agonists, such as e.g. TEGASEROD,

5-HT4 antagonists, such as e.g. PIBOSEROD,

5-HT4 agonists, such as e.g. MOSAPRIDE,

5-HT3-agonists, such as e.g. PUMOSETRAG;

motilin receptor agonists, such as e.g. MITEMCINAL;

cholecystokinin B antagonists, such as e.g. ITRIGLUMIDE, or Z-360; or

cholecystokinin A antagonists, such as e.g. DEXLOXIGLUMIDE;

for simultaneous, sequential, separate or chronologically staggered use in therapy in any order, e.g. to treat or prevent GERD.

Yet another particular embodiment according to the present invention (embodiment a12) to be more emphasized refers to a combination comprising

a first active ingredient which is any acid pump antagonist selected from List C; and

a second active ingredient which refers to any compound or class of compounds of

DOBUPRIDE, KW-5092, KW-5139, R-137696, SR-58611-A, T-1815, Z-338, or CINITAPRIDE; motilin receptor agonists, such as e.g. ALEMCINAL, IDREMCINAL, MITEMCINAL, or SK-896; dopamine D2 receptor antagonists, such as e.g. ITOPRIDE, LEVOSULPIRIDE, METOCLOPRAMIDE, or TICALOPRIDE;

5-HT-partial-)agonists/antagonists, such as e.g.

BIMU-1, CILANSETRON, DAZOPRIDE, E-3620, EM-523, FABESETRON, LINTOPRIDE, LIREXAPRIDE, MOSAPRIDE, PIBOSEROD, PUMOSETRAG, R-137696, RENZAPRIDE, RICASETRON, TICALOPRIDE, Y-36912, YM-114, YM-47813, or ZACOPRIDE;

5-HT4 partial agonists, such as e.g. TEGASEROD;

5-HT4 agonists, such as e.g. PRUCALOPRIDE;

muscarinic M3 antagonists, such as e.g. DARIFENACIN;

kappa opioid receptor agonists, such as e.g. ASIMADOLINE, or FEDOTOZINE; or

dual 5-HT3-antagonists/5-HT4 agonists, such as e.g. BIMU-1, or RENZAPRIDE;

cholecystokinin A antagonists, such as e.g. DEXLOXIGLUMIDE, or ITRIGLUMIDE;

for simultaneous, sequential, separate or chronologically staggered use in therapy in any order, e.g. to treat or prevent gastrointestinal diseases, such as e.g. IBS or GERD.

Yet another particular embodiment according to the present invention (embodiment a13) to be more emphasized refers to a combination comprising

a first active ingredient which is any acid pump antagonist selected from List C; and

a second active ingredient which refers to any compound or class of compounds of compounds which reduce the incidence of transient lower esophageal sphincter relaxation (TLOSR), such as, for example,

GABA-B receptor agonists such as e.g. a compound selected from the group consisting of:

(3-amino-2-fluoropropyl)phosphinic acid,
(R)-(3-amino-2-fluoropropyl)phosphinic acid,
(S)-(3-amino-2-fluoropropyl)phosphinic acid,
(3-amino-2-fluoro-1-methyl-propyl)phosphinic acid,
(3-amino-2-oxopropyl)phosphinic acid,
(S)-(3-amino-2-hydroxypropyl)phosphinic acid,
(R)-(3-amino-2-hydroxypropyl)phosphinic acid,
(3-amino-1-fluoro-2-hydroxypropyl)phosphinic acid,
(3-amino-2-fluoro-propyl)(methyl)phosphinic acid,
(2R)-(3-amino-2-fluoro-propyl)(methyl)phosphinic acid,
(2S)-(3-amino-2-fluoro-propyl)(methyl)phosphinic acid,
(3-amino-2-fluoro-1-methylpropyl)(methyl)phosphinic acid,
(3-amino-1-fluoropropyl)phosphinic acid,
3-[(4-chlorobenzyl)amino]propyl(methyl)phosphinic acid,
3-[1-({3-[hydroxy(oxido)phosphino]propyl}amino)ethyl]benzoic acid acid,
(3-amino-2-fluoropropyl)sulphinic acid,
(2S)-(3-amino-2-fluoropropyl)sulphinic acid,
(2R)-(3-amino-2-fluoropropyl)sulphinic acid,
(2S)-(3-amino-2-hydroxypropyl)sulphinic acid,
(2R)-(3-amino-2-hydroxypropyl)sulphinic acid, and
(3-amino-2-oxopropyl)sulphinic acid,
   or a pharmaceutically acceptable salt, solvate or stereolsomer thereof;
   for simultaneous, sequential, separate or chronologically staggered use in therapy in any order, e.g. to treat or prevent GERD.

Still yet another particular embodiment according to the present invention (embodiment a14) to be in particular emphasized refers to a combination comprising

a first active ingredient which is (7R,8R,9R)-8-hydroxy-7-(2-methoxyethoxy)-2,3-dimethyl-9-phenyl-7,8,9,10-tetrahydro-imidazo[1,2-h][1,7]naphthyridine, or a salt, solvate or solvate of the salt thereof; and

a second active ingredient which is

any 5-HT4-partial-agonist such as e.g. TEGASEROD;

any 5-HT4-agonist such as e.g. MOSAPRIDE or PRUCALOPRIDE;

any 5-HT3 receptor antagonist such as e.g. CILANSETRON, ALOSETRON, RAMOSETRON, AZASETRON, ONDANSETRON, DOLASETRON, GRANISETRON, or TROPISETRON;

any 5-HT4 antagonist such as e.g. PIBOSEROD, or LY-353433;

any dual 5-HT3-antagonist/5-HT4-agonist such as e.g. CISAPRIDE, NOR-CISAPRIDE, (+)NORCISAPRIDE, BIMU1, BIMU8, RENZAPRIDE, ZACOPRIDE, LINTOPRIDE, ITASETRON, FABESETRON, or E-3620;

for simultaneous, sequential, separate or chronologically staggered use in therapy in any order.

Still yet another particular embodiment according to the present invention (embodiment a15) to be in particular emphasized refers to a combination comprising

a first active ingredient which is (7R,8R,9R)-8-hydroxy-7-(2-methoxyethoxy)-2,3-dimethyl-9-phenyl-7,8,9,10-tetrahydro-imidazo[1,2-h][1,7]naphthyridine, or a salt, solvate or solvate of the salt thereof; and

a second active ingredient which is

PRUCALOPRIDE or CILANSETRON, or, in particular,

ALOSETRON, or, in more particular,

TEGASEROD,

for simultaneous, sequential, separate or chronologically staggered use in therapy in any order.

Still yet another particular embodiment according to the present invention (embodiment a16) to be in particular emphasized refers to a combination comprising

a first active ingredient which is (7R,8R,9R)-8-hydroxy-7-(2-methoxyethoxy)-2,3-dimethyl-9-phenyl-7,8,9,10-tetrahydro-imidazo[1,2-h][1,7]naphthyridine, or a salt, solvate or solvate of the salt thereof; and

a second active ingredient which is selected from the group consisting of

TEGASEROD, ALOSETRON, CILANSETRON, PRUCALOPRIDE, ALVIMOPAN, PIBOSEROD and DEXLOXIGLUMIDE;

for simultaneous, sequential, separate or chronologically staggered use in therapy in any order.

Still yet another particular embodiment according to the present invention (embodiment a17) to be in particular emphasized refers to a combination comprising

a first active ingredient which is any acid pump antagonist according to detail a, in particular an acid pump antagonist selected from List A; in more particular selected from List C; and

a second active ingredient which is TEGASEROD, or a salt or a tautomer thereof, such as e.g. TEGASEROD MESYLATE (Zelmac) or MALEATE (Zelnorm);

for simultaneous, sequential, separate or chronologically staggered use in therapy in any order.

Still yet another particular embodiment according to the present invention (embodiment a18) to be in particular emphasized refers to a combination comprising

a first active ingredient which is (7R,8R,9R)-8-hydroxy-7-(2-methoxyethoxy)-2,3-dimethyl-9-phenyl-7,8,9,10-tetrahydro-imidazo[1,2-h][1,7]naphthyridine, or a salt, solvate or solvate of the salt thereof; and

a second active ingredient which refers to any compound or class of compounds of TICALOPRIDE,

5-HT4 antagonists, such as e.g. PIBOSEROD, or LY-353433,

5-HT3 antagonists, such as e.g. YM-114, or CILANSETRON, RAMOSETRON or ALOSETRON,

5-HT4 partial agonists, such as e.g. TEGASEROD,

5-HT4 agonists, such as e.g. PRUCALOPRIDE,

dual 5-HT3 antagonists/5-HT4 agonists, such as e.g. FABESETRON, or E-3620 or RENZAPRIDE;

cholecystokinin A antagonists, such as e.g. DEXLOXIGLUMIDE;

NK-2 antagonists, such as e.g. NEPADUTANT or SAREDUTANT,

NK-3 antagonists, such as e.g. TALNETANT;

kappa opioid receptor agonists, such as e.g. FEDOTOZINE, PTI-901 or ASIMADOLINE;

delta opioid receptor agonists, such as e.g. ALVIMOPAN; or

muscarinic M3 antagonists, such as e.g. ZAMIFENACIN, or (SYOXYBUTININ, J-104135 or DARIFENAZIN;

for simultaneous, sequential, separate or chronologically staggered use in therapy in any order, e.g. to treat or prevent IBS.

Still yet another particular embodiment according to the present invention (embodiment a19) to be in particular emphasized refers to a combination comprising

a first active ingredient which is (7R,8R,9R)-8-hydroxy-7-(2-methoxyethoxy)-2,3-d]methyl-9-phenyl-7,8,9,10-tetrahydro-imidazo[1,2-h][1,7]naphthyridine, or a salt, solvate or solvate of the salt thereof; and

a second active ingredient which refers to any compound or class of compounds of TICALOPRIDE,

5-HT4 partial agonists, such as e.g. TEGASEROD,

5-HT4 antagonists, such as e.g. PIBOSEROD,

5-HT4 agonists, such as e.g. MOSAPRIDE,

5-HT3-agonists, such as e.g. PUMOSETRAG;

motilin receptor agonists, such as e.g. MITEMCINAL;

cholecystokinin B antagonists, such as e.g. ITRIGLUMIDE, or Z-360; or

cholecystokinin A antagonists, such as e.g. DEXLOXIGLUMIDE;

for simultaneous, sequential, separate or chronologically staggered use in therapy in any order, e.g. to treat or prevent GERD.

Still yet another particular embodiment according to the present invention (embodiment a20) to be in particular emphasized refers to a combination comprising

a first active ingredient which is (7R,8R,9R)-8-hydroxy-7-(2-methoxyethoxy)-2,3-dimethyl-9-phenyl-7,8,9,10-tetrahydro-imidazo[1,2-h][1,7]naphthyridine, or a salt, solvate or solvate of the salt thereof; and

a second active ingredient which refers to any compound or class of compounds of DOBUPRIDE, KW-5092, KW-5139, R-137696, SR-58611-A, T-1815, Z-338, or CINITAPRIDE;

motilin receptor agonists, such as e.g. ALEMCINAL, IDREMCINAL, MITEMCINAL, or SK-896;

dopamine D2 receptor antagonists, such as e.g. ITOPRIDE, LEVOSULPIRIDE, METOCLOPRAMIDE, or TICALOPRIDE;

5-HT-(partial-)agonists/antagonists, such as e.g.

BIMU-1, CILANSETRON, DAZOPRIDE, E-3620, EM-523, FABESETRON, LINTOPRIDE, LIREXAPRIDE, MOSAPRIDE, PIBOSEROD, PUMOSETRAG, R-137696, RENZAPRIDE, RICASETRON, TICALOPRIDE, Y-36912, YM-114, YM47813, or ZACOPRIDE;

5-HT4 partial agonists, such as e.g. TEGASEROD;

5-HT4 agonists, such as e.g. PRUCALOPRIDE;

muscarinic M3 antagonists, such as e.g. DARIFENACIN;

kappa oploid receptor agonists, such as e.g. ASIMADOLINE, or FEDOTOZINE; or

dual 5-HT3-antagonists/5-HT4 agonists, such as e.g. BIMU-1, or RENZAPRIDE;

cholecystokinin A antagonists, such as e.g. DEXLOXIGLUMIDE, or ITRIGLUMIDE;

for simultaneous, sequential, separate or chronologically staggered use in therapy in any order, e.g. to treat or prevent gastrointestinal diseases, such as e.g. IBS or GERD.

Still yet another particular embodiment according to the present invention (embodiment a21) to be in particular emphasized refers to a combination comprising

a first active ingredient which is (7R,8R,9R)-8-hydroxy-7-(2-methoxyethoxy)-2,3-dimethyl-9-phenyl-7,8,9,10-tetrahydro-imidazo[1,2-h][1,7]naphthyridine, or a salt, solvate or solvate of the salt thereof; and

a second active ingredient which refers to any compound or class of compounds of

compounds which reduce the incidence of transient lower esophageal sphincter relaxation (TLOSR), such as, for example,

GABA-B receptor agonists such as e.g. a compound selected from the group consisting of:

(3-amino-2-fluoropropyl)phosphinic acid,
(R)-(3-amino-2-fluoropropyl)phosphinic acid,
(S)-(3-amino-2-fluoropropyl)phosphinic acid,
(3-amino-2-fluoro-1-methyl-propyl)phosphinic acid,
(3-amino-2-oxopropyl)phosphinic acid,
(S)-(3-amino-2-hydroxypropyl)phosphinic acid,
(R)-(3-amino-2-hydroxypropyl)phosphinic acid,
(3-amino-1-fluoro-2-hydroxypropyl)phosphinic acid,
(3-amino-2-fluoro-propyl)(methyl)phosphinic acid,
(2R)-(3-amino-2-fluoro-propyl)(methyl)phosphinic acid,
(2S)-(3-amino-2-fluoro-propyl)(methyl)phosphinic acid,
(3-amino-2-fluoro-propyl)methylpropyl)(methyl)phosphinic acid,
(3-amino-1-fluoropropyl)phosphinic acid,
3-[(4-chlorobenzyl)amino]propyl(methyl)phosphinic acid,
3-[1-({3-[hydroxy(oxido)phosphino]propyl}amino)ethyl]benzoic acid acid,
(3-amino-2-fluoropropyl)sulphinic acid,
(2S)-(3-amino-2-fluoropropyl)sulphinic acid,
(2R)-(3-amino-2-fluoropropyl)sulphinic acid,
(2S)-(3-amino-2-hydroxypropyl)sulphinic acid,
(2R)-(3-amino-2-hydroxypropyl)sulphinic acid, and
(3-amino-2-oxopropyl)sulphinic acid,
   or a pharmaceutically acceptable salt, solvate or stereolsomer thereof;
   for simultaneous, sequential, separate or chronologically staggered use in therapy in any order, e.g. to treat or prevent GERD.

Compounds, which modify gastrointestinal motility, to be emphasized in another embodiment of the present invention (embodiment b) as particularly useful to be employed in combination with acid pump antagonists, are active agents selected from the following active agent classes:

5-HT-(partial-)agonists/antagonists (such as, e.g. 5-HT2-, 5-HT3- and 5-HT4-(partial-)agonists/antagonists, in particular 5-HT3-antagonists, 5-HT4-agonists or 5-HT4-antagonists), muscarinic (e.g. muscarinic M3) antagonists, opioid receptor agonists (e.g. delta opioid receptor agonists or, in particular, kappa opioid receptor agonists), dopamine receptor antagonists (in particular dopamine D2 receptor antagonists), cholecystokinin A antagonists, motilin agonists (motilides), NMDA-receptor antagonists, non-NMDA glutamate receptor antagonists, neurokinin antagonists (in particular NK-1, NK-2 or NK-3 antagonists), alpha-2 adrenoceptor agonists, corticotropin releasing factor antagonists, somatostatin agonists, NO-synthase inhibitors, GABA (in particular GABA-B) receptor agonists/partial agonists or active agents which reduce the incidence of transient lower esophageal sphincter relaxation (TLOSR), and/or gastroprokinetics, antiemetics or antispasmodics.

In the context of embodiment b, compounds, which modify gastrointestinal motility, to be more emphasized in the meaning of the present invention as particularly useful to be employed in combination with acid pump antagonists, are active agents for use in therapy of irritable bowel syndrome (IBS) selected from the following active agent classes:

5-HT-(partial-)agonists/antagonists (such as, e.g. 5-HT3-antagonists, 5-HT4-agonists or 5-HT4 antagonists), cholecystokinin A antagonists, muscarinic M3 antagonists, kappa opioid receptor ago nists, motilin agonists (motilides), delta opioid receptor agonists, dopamine receptor antagonists, neurokinin antagonists (in particular NK-1, NK-2 or NK-3 antagonists), NMDA-receptor antagonists, alpha-2 adrenoceptor agonists or corticotropin releasing factor antagonists.

Yet in the context of embodiment b, further compounds, which modify gastrointestinal motility, to be more emphasized in the meaning of the present invention as particularly useful to be employed in combination with acid pump antagonists, are active agents for use in therapy of gastro-esophageal reflux disease (GERD) selected from the following active agent classes:

motilin agonists (motilides), 5-HT-(partial-)agonists/antagonists (such as, e.g. 5-HT3-antagonists, 5-HT4-agonists or 5-HT4-antagonists), muscarinic antagonists, opioid agonists/partial agonists, NMDA-receptor antagonists, non-NMDA glutamate receptor antagonists, somatostatin agonists, NO-synthase inhibitors, GABA (in particular GABA-B) receptor agonists or active agents which reduce the incidence of transient lower esophageal sphincter relaxation (TLOSR).

In the connection of embodiment b and/or a, exemplary compounds, which modify gastrointestinal motility, to be emphasized within the meaning of the present invention in a special facet include active agents for use in therapy of IBS or GERD, or for use as gastroprokinetics or antiemetics, such as, for example without being restricted thereto,

(S)-OXYBUTININ, ALEMCINAL, ALIZAPRIDE, ALOSETRON, ALTINICLINE, ALVIMOPAN, APREPITANT, AZASETRON, BATANOPRIDE, BROMOPRIDE, CILANSETRON, CINITAPRIDE, CISAPRIDE, CLEBOPRIDE, DARIFENACIN, DAZOPRIDE, DEXANABINOL, DEXLOXIGLUMIDE, DIFENIDOL, DOBUPRIDE, DOMPERIDONE, E-3620, EXEPANOL, FABESETRON, FEDOTOZINE, GRANISETRON, INDISETRON, ITASETRON, ITOPRIDE, KW-5092, KW-5139, LERISETRON, LEVOSULPIRIDE, LINTOPRIDE, LIREXAPRIDE, LY-353433, METOCLOPRAMIDE, MITEMCINAL, MOSAPRIDE, ONDANSETRON, PALONOSETRON, PIBOSEROD, PRUCALOPRIDE, R-137696, RAMOSETRON, RENZAPRIDE, RS-25259-197, SR-58611-A, TEGASEROD, TIAPRIDE, TICALOPRIDE, TRIMEBUTINE, TROPISETRON, VOFOPITANT, Z-338 or ZACOPRIDE.

Yet in the connection of embodiment b and/or a, exemplary compounds, which modify gastrointestinal motility, to be emphasized within the meaning of the present invention in a further special facet include active agents for use in therapy of IBS or GERD, such as, for example without being restricted thereto,

ALOSETRON, ALVIMOPAN, CILANSETRON, DARIFENACIN, DEXLOXIGLUMIDE, E-3620, FABESETRON, LINTOPRIDE, LY-353433, MITEMCINAL, (S)-OXYBUTININ, PIBOSEROD, TEGASEROD, TICALOPRIDE or TRIMEBUTINE.

Yet in the connection of embodiment b and/or a, exemplary compounds, which modify gastrointestinal motility, to be emphasized within the meaning of the present invention in a yet further facet include suitably

ALEMCINAL, ASIMADOLINE, BACLOFEN, BIPERIDEN, CILANSETRON, CINITAPRIDE, CISAPRIDE, CLEBOPRIDE, DARIFENACIN, DAZOPRIDE, DIFENIDOL, DOBUPRIDE, E-3620, EM-523, FABESETRON, FEDOTOZINE, GABAPENTIN, IDREMCINAL, ITOPRIDE, KW-5092, KW-5139, LEVOSULPIRIDE, LINTOPRIDE, LIREXAPRIDE, MEBEVERINE, METOCLOPRAMIDE, MITEMCINAL, MOSAPRIDE, NITRAQUAZONE, PAZINACLONE, PIBOSEROD, PRIDINOL, PROCYCLIDINE, PRUCALOPRIDE, PUMOSETRAG, R-137696, RENZAPRIDE, RICASETRON, ROLIPRAM, SK-896, SL-65.1498, SR-58611-A, T-1815, TEGASEROD, TIBENELAST, TICALOPRIDE, TRIHEXYPHENIDYL, Y-36912, YM-114, YM47813, Z-338 and ZACOPRIDE.

Yet in the connection of embodiment b and/or a, exemplary compounds, which modify gastrointestinal motility, to be emphasized within the meaning of the present invention in a still yet further special facet include suitably

ALEMCINAL, ALVIMOPAN, CINITAPRIDE, DEXLOXIGLUMIDE, DOBUPRIDE, FEDOTOZINE, KW-5092, KW-5139, ITOPRIDE, LIREXAPRIDE, MITEMCINAL, PIBOSEROD, PRUCALOPRIDE, R-137696, RENZAPRIDE, SR-58611-A, T-1815, TEGASEROD, TICALOPRIDE and Z-338.

As used throughout, classes of compounds, which are mentioned as combination partners according to this invention, are used for describing each and every member that is within this class. Any member within this class can be selected as combination partner according to this invention.

Any or all of the listed combination partners as defined in this invention may be suitable to be used in the combination therapy or in the combinations or compositions according to the present invention.

The expression “gastrointestinal diseases” comprises diseases or disorders of the gastrointestinal tract known to the person skilled in the art. In this context, gastrointestinal motility disorders, disorders of gastric emptying, bowel disorders, esophageal diseases, gastrointestinal inflammatory diseases (such as inflammatory bowel disease), and gastrointestinal diseases associated with inflammatoric attendant phenomenons are to be emphasized, as well as dyspepsia, vomiting and those diseases mentioned below.

Particularly emphasized are hereby the gastro-esophageal reflux disease (GERD) and the irritable bowel syndrome (IBS), and the symptoms associated therewith.

These “gastrointestinal diseases” or conditions are characterized by or associated with altered gastrointestinal motility, sensitivity, secretion and/or infections and can be from organic, non-organic or functional origins.

In a more detailed facet of “gastrointestinal diseases” as used herein, diseases which can be treated or prevented by inhibition of the incidence of transient lower esophageal sphincter relaxation (TLOSR) are to be mentioned. Accordingly, diseases which can be treated or prevented by inhibition of transient lower esophageal sphincter relaxations (TLOSRs) are known to the person skilled in the art; Exemplarily can be mentioned in this context: GERD, regurgitation, esophagitis, asthma (such as reflux-related or non reflux related asthma), failure to thrive and laryngitis.

Thus, in the scope of this invention, the combination of certain acid pump antagonists and compounds, which modify gastrointestinal motility, as described herein can widen and/or potentiate the use of acid pump antagonists in therapy, prophylaxis or amelioration of gastrointestinal diseases, such as those mentioned herein, in particular IBS or, in more particular, GERD.

In this context and in a more detailed facet thereof, the combination of certain acid pump antagonists and compounds, which inhibit transient lower esophageal sphincter relaxations (TLOSRs), as described herein can widen and/or potentiate the use of acid pump antagonists in therapy or prophylaxis of diseases which can be treated, prevented or ameliorated by inhibition of transient lower esophageal sphincter relaxations (TLOSRs), such as those mentioned herein, in particular GERD, in more particular severe GERD (grade III and IV).

The wording of “gastro-esophageal reflux disease” and “GERD”, as well as “irritable bowel syndrome” and “IBS” are herein defined in accordance with the meaning known to the skilled person including all forms or manifestations thereof. Thus, for example, “gastro-esophageal reflux disease” and “GERD” include, without being limited to, erosive and non-erosive GERD, heartburn and other symptoms associated with GERD. Accordingly, “irritable bowel syndrome” and “IBS” include, without being limited to,

symptoms associated with disordered function involving altered gastrointestinal motility, sensitivity and secretion involving the small intestine and large bowel, such as e.g. variable degrees of abdominal pain, constipation, bloating or diarrhea without bowel inflammation.

It is habitual to the person skilled in the art to decide on the base of his/her expert knowledge and/or of relevant prior art what is the meaning of the terms “agonists”, “antagonists” or “inhibitors” as used in their respective context in this invention.

The person skilled in the art knows how to assess whether a compound meets the functional criteria of the active agent classes mentioned herein as groups of compounds, which modify gastrointestinal motility. Therefor, for example, the person skilled in the art can use test systems described in the art and/or he/she can consult art-known databases, monographs, handbooks or public literature.

As a first aspect of the present invention (aspect 1), this invention relates to the combined use of certain acid pump antagonists and compounds, which modify gastrointestinal motility, in the treatment of gastrointestinal diseases, in particular gastro-esophageal reflux disease (GERD) or irritable bowel syndrome (IBS).

In a further aspect (aspect 2), this invention relates to the combined use of certain acid pump antagonists and compounds which modify gastrointestinal motility, particularly GABA-B receptor agonists, to reduce the incidence of transient lower esophageal sphincter relaxation (TLOSR).

An alternative aspect of the present invention (aspect 3) relates to the combined use of certain acid pump antagonists and compounds, which modify gastrointestinal motility, in the improved treatment of altered gastrointestinal motility, sensitivity and/or secretion and/or abdominal disorders including both functional and organic diseases, such as, for example, in the treatment of chronic symptoms of dyspepsia and diseases associated herewith, such as, for example, GERD, duodenal ulcer or gastric ulcer and other diagnoses (e.g. functional/non-ulcerative dyspepsia, gallbladder or liver diseases).

A further aspect (aspect 4) of the present invention relates to the combined use of certain acid pump antagonists and compounds, which modify gastrointestinal motility, to normalize, stabilize and/or regulate altered gastrointestinal motility, sensitivity and/or secretion in therapy.

A further aspect (aspect 5) of the present invention relates to the combined use of certain acid pump antagonists and compounds, which modify gastrointestinal motility, to obtain a particularly enhanced treatment response for altered gastrointestinal motility, sensitivity and/or secretion and/or abdominal disorders, in particular in patients suffering from GERD, and/or to obtain a particularly enhanced reduction of gastrointestinal pain and other symptoms normally associated with disturbed/altered gastrointestinal motility, sensitivity and/or secretion.

A further aspect (aspect 6) of the present invention is the use of certain acid pump antagonists and compounds, which modify gastrointestinal motility, in the manufacture of pharmaceutical compositions for the treatment of gastrointestinal diseases, in particular gastro-esophageal reflux disease (GERD) or irritable bowel syndrome (IBS).

A further aspect (aspect 7) of the present invention is the use of at least one certain acid pump antagonist and at least one compound, which modify gastrointestinal motility, in the manufacture of a combination for the treatment of gastrointestinal diseases, in particular gastro-esophageal reflux disease (GERD) or irritable bowel syndrome (IBS).

A further aspect (aspect 8) of the present invention is the use of at least one certain acid pump antagonist and at least one compound, which modify gastrointestinal motility, in the manufacture of a combination for the inhibition of transient lower esophageal sphincter relaxations (TLOSRs).

A further aspect (aspect 9) of the present invention is the use of a pharmaceutical composition or combination according to this invention in the manufacture of a pharmaceutical product for the treatment or prevention of gastrointestinal motility disorders.

A further aspect of the present invention (aspect 10) is the use of a pharmaceutical composition, pharmaceutical product, formulation, preparation, combination, commercial package or kit according to the invention in the manufacture of a medicament for use in the treatment of gastrointestinal diseases, in particular gastro-esophageal reflux disease (GERD) or irritable bowel syndrome (IBS).

A further aspect of the present invention (aspect 11) is the simultaneous, separate or sequential coadministration of one or more certain acid pump anatagonists with one or more compounds, which modify gastrointestinal motility, to treat gastrointestinal diseases, in particular gastro-esophageal reflux disease (GERD) or irritable bowel syndrome (IBS).

A further aspect of the present invention (aspect 12) is a method for treatment of gastrointestinal diseases, in particular gastro-esophageal reflux disease (GERD) or irritable bowel syndrome (IBS), comprising administering an effective amount of one or more certain acid pump anatagonists simultaneously, separately or sequentially with one or more compounds, which modify gastrointestinal motility, to a mammal, preferably a human, in need thereof.

A further aspect of the present invention (aspect 13) is a method for treatment of gastrointestinal diseases, in particular gastro-esophageal reflux disease (GERD) or irritable bowel syndrome (IBS), comprising administering a pharmaceutical composition or combination according to this invention to a mammal, preferably a human, in need thereof.

A further aspect of the present invention (aspect 14) is a method for the inhibition of transient lower esophageal sphincter relaxation TLOSRs) comprising administering an effective amount of one or more certain acid pump anatagonists simultaneously, separately or sequentially with one or more compounds, which modify gastrointestinal motility, in particular one or more GABA B receptor agonists, to a mammal, preferably a human, in need thereof.

In a special aspect (aspect 15), this invention relates to the combined use of certain acid pump antagonists and compounds, which reduce the incidence of transient lower esophageal sphincter relaxation (TLOSR), in the treatment of gastrointestinal diseases, in particular gastro-esophageal reflux disease (GERD).

A further special aspect of the present invention (aspect 16) is the use of certain acid pump antagonists and compounds, which reduce the incidence of transient lower esophageal sphincter relaxation (TLOSR), in the manufacture of pharmaceutical compositions for the treatment of gastrointestinal diseases, in particular gastro-esophageal reflux disease (GERD).

A further special aspect of the present invention (aspect 17) is the simultaneous, separate or sequential coadministration of one or more certain acid pump anatagonists with one or more compounds, which reduce the incidence of transient lower esophageal sphincter relaxation TLOSR), to treat gastrointestinal diseases, in particular gastro-esophageal reflux disease (GERD).

A further special aspect of the present invention (aspect 18) is a method for treatment of gastrointestinal diseases, in particular gastro-esophageal reflux disease (GERD), comprising administering an effective amount of one or more certain acid pump anatagonists simultaneously, separately or sequentially with one or more compounds, which reduce the incidence of transient lower esophageal sphincter relaxation TLOSR), to a mammal, preferably a human, in need thereof.

A further aspect of the present invention (aspect 19) is a preferably orally applicable pharmaceutical composition for simultaneous administration comprising, in admixture, a first active ingredient, which is at least one certain acid pump antagonist, and a second active ingredient, which is at least one compound, which modifies gastrointestinal motility, to treat gastrointestinal diseases, in particular gastro-esophageal reflux disease (GERD) or irritable bowel syndrome (IBS) in a mammal, preferably a human.

A further aspect of the present invention (aspect 20) is a composition comprising a first active ingredient, which is at least one certain acid pump antagonist, and a second active ingredient, which is at least one compound, which modifies gastrointestinal motility, for simultaneous, sequential or separate use in therapy in any order.

A further aspect of the present invention (aspect 21) is a preferably orally applicable pharmaceutical composition in unit dosage comprising at least one certain acid pump antagonist together with at least one compound, which modifies gastrointestinal motility, for use in therapy, e.g. to treat gastrointestinal diseases, in particular gastro-esophageal reflux disease (GERD) or irritable bowel syndrome (IBS) in a mammal.

A further aspect of the present invention (aspect 22) is a pharmaceutical composition comprising at least one certain acid pump antagonist together with at least one compound, which modifies gastrointestinal motility, wherein the acid pump antagonist and the compound, which modifies gastrointestinal motility, are administered in a single dosage form, such that the acid pump antagonist and the compound, which modifies gastrointestinal motility, are physically separated from each other.

A further aspect of the present invention (aspect 23) is a pharmaceutical composition comprising, in admixture, a first active ingredient, which is at least one certain acid pump antagonist, and a second active ingredient, which is at least one compound, which modifies gastrointestinal motility.

A further aspect of this invention (aspect 24) is a pharmaceutical composition comprising:
(a) a pharmaceutically effective amount of at least one certain acid pump antagonist; and
(b) a pharmaceutically effective amount of at least one compound, which modifies gastrointestinal motility.

A further aspect of this invention (aspect 25) is a pharmaceutical composition comprising:
(a) a pharmaceutically effective amount of at least one certain acid pump antagonist, and
(b) a pharmaceutically effective amount of at least one compound, which modifies gastrointestinal motility,
wherein component (a) and component (b) are maintained in the same delivery vehicle.

A further aspect of this invention (aspect 26) is a pharmaceutical composition comprising:
(a) a pharmaceutically effective amount of at least one certain acid pump antagonist, and
(b) a pharmaceutically effective amount of at least one compound, which modifies gastrointestinal motility,
wherein component (a) and component (b) are maintained in different delivery vehicles.

A further aspect of the present invention (aspect 27) is a preferably orally applicable pharmaceutical formulation comprising a first active ingredient, which is a certain acid pump antagonist, a second active ingredient, which is at least one compound, which modifies gastrointestinal motility, and a pharmaceutically acceptable carrier, diluent, adjuvant, auxiliary or excipient for use in therapy, e.g. to treat gastrointestinal diseases, in particular gastro-esophageal reflux disease (GERD) or irritable bowel syndrome (IBS) in a mammal, especially a human.

A further aspect of the present invention (aspect 28) is a pharmaceutical composition comprising a first active ingredient, which is a certain acid pump antagonist, a second active ingredient, which is at least one compound, which modifies gastrointestinal motility, and one or more pharmaceutically acceptable carriers, diluents, adjuvants, auxiliaries or excipients.

A further aspect of the present invention (aspect 29) is a first pharmaceutical formulation comprising at least one certain acid pump antagonist and a pharmaceutically acceptable carrier or diluent, and a second pharmaceutical formulation comprising a compound, which modifies gastrointestinal motility, and a pharmaceutically acceptable carrier or diluent.

A further aspect of the present invention (aspect 30) is a combination comprising a certain acid pump antagonist and at least one compound, which modifies gastrointestinal motility, for simultaneous, sequential or separate use in therapy, e.g. to treat gastrointestinal diseases, in particular gastroesophageal reflux disease (GERD) or irritable bowel syndrome (IBS) in a mammal, especially a human.

A further aspect of the present invention (aspect 31) is a combination such as, for example, a combined preparation, a kit-of-parts or a composition, comprising at least one certain acid pump antagonist and at least one compound, which modifies gastrointestinal motility, and, optionally, at least one pharmaceutically acceptable carrier or diluent, for simultaneous, sequential, separate or chronologically staggered use in therapy, and/or for use as single, combined or separate unit dosage forms in therapy, and/or for use as fixed or non-fixed combination in therapy, and/or for use as admixture in therapy, e.g. to treat gastrointestinal diseases, in particular gastro-esophageal reflux disease (GERD) or irritable bowel syndrome (IBS) in a mammal, especially a human.

A further special aspect of the present invention (aspect 32) is a pharmaceutical product comprising, in combination, a first active ingredient, which is at least one certain acid pump antagonist, and a second active ingredient, which is at least one compound, which modifies gastrointestinal motility, for simultaneous, sequential or separate use in therapy.

A further aspect of the present invention (aspect 33) is a pharmaceutical product comprising, in combination, a preparation of a first active ingredient, which is at least one certain acid pump antagonist, and a preparation of a second active ingredient, which is at least one compound, which modifies gastrointestinal motility, for simultaneous, sequential or separate use in therapy, e.g. to treat gastrointestinal diseases, in particular gastro-esophageal reflux disease (GERD) or irritable bowel syndrome (IBS) in a mammal, especially a human.

A further aspect of the present invention (aspect 34) is a pharmaceutical preparation comprising a first active ingredient, which is at least one certain acid pump antagonist, a second active ingredient, which is at least one compound, which modifies gastrointestinal motility, and one or more pharmaceutically acceptable carriers, diluents, adjuvants, auxiliaries or excipients.

A further aspect of the present invention (aspect 35) is a commercial package comprising a first active ingredient, which is at least one certain acid pump antagonist, and a second active ingredient, which is at least one compound, which modifies gastrointestinal motility, together with standard packaging material, and together with instructions for simultaneous, sequential or separate use in therapy.

A further aspect of the present invention (aspect 36) is a commercial package comprising at least one certain acid pump antagonist as active ingredient together with instructions for simultaneous, sequential or separate use with a compound, which modifies gastrointestinal motility.

A further aspect of the present invention (aspect 37) is a commercial package comprising at least one compound, which modifies gastrointestinal motility, as active ingredient(s) together with instructions for simultaneous, sequential or separate use with at least one certain acid pump antagonist.

A further aspect of the present invention (aspect 38) is a kit comprising at least one dosage unit of a certain acid pump antagonist as well as at least one dosage unit of at least one compound, which modifies gastrointestinal motility, for simultaneous, sequential or separate use in therapy. Optionally, abovementioned kit can be provided with instructions for use.

A further aspect of the present invention (aspect 39) is a kit comprising a preparation of a first active ingredient, which is at least one certain acid pump antagonist, a preparation of a second active ingredient, which is at least one compound, which modifies gastrointestinal motility, and instructions for simultaneous, sequential or separate administration of the preparations to a patient in need thereof.

A further special aspect of the present invention (aspect 40) is a preferably orally applicable pharmaceutical composition for simultaneous administration comprising, in admixture, a first active ingredient, which is at least one certain acid pump antagonist, and a second active ingredient, which is at least one compound, which reduces the incidence of transient lower esophageal sphincter relaxation (TLOSR), to treat gastrointestinal diseases, in particular gastro-esophageal reflux disease (GERD) in a mammal, preferably a human.

A further special aspect of the present invention (aspect 41) is a combination or composition comprising a first active ingredient, which is at least one certain acid pump antagonist, and a second active ingredient, which is at least one compound, which reduces the incidence of transient lower esophageal sphincter relaxation (TLOSR), for simultaneous, sequential or separate use in therapy in any order.

A further special aspect of the present invention (aspect 42) is a pharmaceutical product comprising, in combination, a first active ingredient, which is at least one certain acid pump antagonist, and a second active ingredient, which is at least one compound, which reduces the incidence of transient lower esophageal sphincter relaxation (TLOSR), for simultaneous, sequential or separate use in therapy.

A further special aspect of the present invention (aspect 43) is a commercial package comprising a first active ingredient, which is at least one certain acid pump antagonist, and a second active ingredient, which is at least one compound, which reduces the incidence of transient lower esophageal sphincter relaxation (TLOSR), together with instructions for simultaneous, sequential or separate use in therapy.

A further special aspect of the present invention (aspect 44) is a commercial package comprising at least one certain acid pump antagonist as active ingredient together with instructions for simultaneous, sequential or separate use with a compound, which reduces the incidence of transient lower esophageal sphincter relaxation (TLOSR).

A further special aspect of the present invention (aspect 45) is a commercial package comprising at least one compound, which reduces the incidence of transient lower esophageal sphincter relaxation (TLOSR), as active ingredient together with instructions for simultaneous, sequential or separate use with at least one certain add pump antagonist.

A further special aspect of the present invention (aspect 46) is a kit comprising a preparation of a first active ingredient, which is at least one certain acid pump antagonist, a preparation of a second active ingredient, which is at least one compound, which reduces the incidence of transient lower esophageal sphincter relaxation (TLOSR), and instructions for simultaneous, sequential or separate administration of the preparations to a patent in need thereof.

In the aforementioned aspects 1 to 46 of the present invention the expressions “certain acid pump antagonist”, and “compound, which reduces the incidence of transient lower esophageal sphincter relaxation” and “compound, which modifies gastrointestinal motility” refer respectively to those compounds or compound classes defined for these expressions in this invention.

Within the meaning of this invention, is to be understood, that any compound or group of compounds which falls under the definition of the term “certain acid pump antagonist” given herein can be combined with any compound or group of compounds which falls under the definition of the term “compound, which modifies gastrointestinal motility” given herein, under the provisio that the teaching anticipated by prior art is thereof disclaimed.

In particular is to be noted in this context that any compound or group of compounds which falls under the definition of the term “certain acid pump antagonist” according to detail a as defined above can be combined with any compound or group of compounds which falls under the definition of the term “compound, which modifies gastrointestinal motility” given herein.

Within the meaning of this invention, is also to be understood, that any compound or group of compounds which falls under the definition of the term “certain acid pump antagonist” given herein can be combined with any compound or group of compounds which falls under the definition of the term “compound, which reduces the incidence of transient lower esophageal sphincter relaxation” given herein, under the provisio that the teaching anticipated by prior art is thereof disclaimed.

Yet in particular is to be noted in this context that any compound or group of compounds which falls under the definition of the term “certain acid pump antagonist” according to detail a as defined above can be combined with any compound or group of compounds which falls under the definition of the term “compound, which reduces the incidence of transient lower esophageal sphincter relaxation” given herein.

Within the meaning of this invention the terms “use”, “administration”, “coadministration” or “administering” refer preferably to oral application. However in some cases, parenteral (e.g. intravenious), subcutaneous or rectal application can be also advantageous.

The dosage of the active compounds is in a customary order of magnitude comparable with the monodosage, whereby, due to the additive and/or superadditive synergism of the single effects, the relevant doses of the active compounds in the combined dosage can be reduced compared to norm, or whereby—while maintaining the customary doses of the single components—a surprisingly higher and prolonged effect is obtained.

In general, it has proven advantageous in human medicine to administer acid pump antagonists in the case of oral administration in a daily dose from approximately 0.01 to approximately 20, preferably 0.05 to 5, in particular 0.1 to 1.5, in more particular 0.1 to 0.5, mg/kg of body weight, if appropriate in the form of several, preferably 1 to 4, individual doses to achieve the desired result. In the case of parenteral treatment, similar or (in particular in the case of intravenous administration of the active compounds), as a rule, lower doses can be used. The optimal dose and manner of administration of the active compounds necessary in each case can easily be determined by any person skilled in the art on the basis of his/her expert knowledge.

The person skilled in the art is aware on the base of his expert knowledge of the total daily dosage of the compounds, which modify gastrointestinal motility, and of the compounds, which reduce the incidence of transient lower esophageal sphincter relaxation (TLOSR), comprised in the above-mentioned (pharmaceutical) compositions, pharmaceutical products, preparations, formulations, combinations, commercial packages or kits according to this invention. Said total daily dosage can vary within a wide range.

In this context, for more detailed example, compositions according to this invention comprising a first active ingredient, which is an acid pump antagonist, and a second active ingredient, which is a 5-HT4-(partial-)agonist/antagonist (e.g. tegaserod or its salt), may be administered in a molar ratio having a range of from about 0.01 to 1000 for the acid pump antagonist to a range of from about 0.01 to about 2 for the 5-HT-(partial-)agonist/antagonist. As an example, the molar ratio for the acid pump antagonist to the 5-HT4-(partial-)agonist/antagonist is about 1000:1 (acid pump antagonist to 5-HT4-(partial)agonist/antagonist). As a more specific example, the molar ratio for the acid pump antagonist to the 5-HT4-(partial-)agonist/antagonist may be about 1000:1, 500:1, 200:1, 100:1, 20:1, 5:1, 1:1, 1:5, 1:20, 1:100. The total daily dose range, which comprises the above described molar ratio, may be administered in a range of from about 0.01 mg to about 1000 mg. The daily dose range may be about 800 mg, 600 mg, 400 mg, 200 mg, 100 mg, 50 mg, 20 mg, 10 mg, 5 mg, 1 mg, 0.1 mg or 0.01 mg. Suitably, a daily dose range should be between about 0.5 mg to about 100 mg, while more suitably, a daily dose range should be between about 5 mg to about 75 mg. The doses can be administered once daily or two times a day. In managing the patient, the therapy should be initiated at a lower dose and increased depending on patient response, whereby the person skilled in the art knows how and when to interrupt, adjust or terminate therapy in conjunction with individual patient response. As it is customary per se to the person skilled in the art, the skilled person knows on the base of his/her expert knowledge that it may be necessary to use dosages outside these abovementioned ranges.

The person skilled in the art is familiar, on the basis of his/her knowledge, with carriers, diluents, adjuvants, auxiliaries or excipients which are suitable for the desired pharmaceutical formulations and/or preparations. Beside solvents, gel-forming agents, suppository bases, tablet auxiliaries and other active carriers, it is possible to use, for example, antioxidants, dispersants, emulsifiers, antifoams, flavor corrigents, preservatives, solubilizers, colorants or, in particular, permeation promoters and complexing agents (e.g. cyclodextrines).

In medicines, the active compounds are preferably employed in the form of tablets, coated tablets, capsules, suppositories, patches, emulsions, suspensions, gels or solutions, the active compound content advantageously being between 0.1 and 95%. Thus, for example with regard to the desired mode and site of action, the person skilled in the art can develop, on the basis of his/her knowledge, by appropriate choice of the excipients and the auxiliaries different galenic forms precisely tailored to the active ingredient(s) (such as, for example, retard forms or gastric acid resistant forms).

A medicament, a combination or a pharmaceutical composition according to this invention can refer to a combination comprising both the said tricyclic imidazo[1,2-a]pyridine compound and the other active ingredient in a fixed combination (fixed unit dosage form), or a medicament pack comprising the two active ingredients as discrete separate dosage forms. In case of a medicament pack comprising the two active ingredients, the active ingredients are preferably packed into blister cards which are suited for improving compliance.

Each blister card preferably contains the medicaments to be taken on one day of treatment. If the medicaments are to be taken at different times of day, the medicaments can be disposed in different sections on the blister card according to the different ranges of times of day at which the medicaments are to be taken (for example morning and evening or morning, midday and evening). The blister cavities for the medicaments to be taken together at a particular time of day are accommodated in the respective range of times of day. The various times of day are, of course, also put on the blister in a clearly visible way. It is also possible, of course, for example to indicate a period in which the medicaments are to be taken, for example stating the times.

The daily sections may represent one line of the blister card, and the times of day are then identified in chronological sequence in this column.

Medicaments which must be taken together at a particular time of day are placed together at the appropriate time on the blister card, preferably a narrow distance apart, allowing them to be pushed out of the blister easily, and having the effect that removal of the dosage form from the blister is not forgotten.

Having described the invention in detail, the scope of the present invention is not limited only to those described characteristics. As will be apparent to persons skilled in the art, modifications, variations and adaptations to the above-described invention can be made on the base of the disclosure (e.g. the explicite, implicite or inherent disclosure) of the present invention without departing from the spirit and scope of this invention.

It is to be understood that the invention covers—unless otherwise noted—all possible combinations of single characteristics, aspects, facets, details or embodiments of the invention as described herein.

The term TLOSR is used herein synonymically to TLESR (i.e. transient lower esophageal sphincter relaxation).

All patents and patent applications referred to herein are incorporated by reference into the specification of the present invention in their entirety for all purposes.

As exemplary and illustrative acid pump antagonists useful within the meaning of this invention each and every compound listed expressis verbis as compound 1 to 17 in the List C of this invention, as well as the salts, solvates and solvates of the salts thereof, may be mentioned, without restricting the present invention thereto.

In a particular detail, Soraprazan, as well as the salts, solvates and solvates of the salts thereof, can be mentioned exemplarily and illustratively as acid pump antagonist useful within the meaning of this invention, but without restricting this invention thereto.

As exemplary and illustrative compounds, which modify gastrointestinal motility, useful within the meaning of this invention 5-HT4-partial-agonists (namely e.g. TEGASEROD), 5-HT4-agonists (namely e.g. PRUCALOPRIDE), 5-HT4-antagonists (namely e.g. PIBOSEROD), 5-HT3-antagonists (namely e.g. CILANSETRON) or dual 5-HT3-antagonists/5-HT4-agonists (namely e.g. (+)-NOR-CISAPRIDE) may be independently mentioned, without restricting the present invention thereto.

In a particular detail, TEGASEROD or a salt or tautomer thereof, such as e.g. Zelmac or Zelnorm, may be mentioned exemplarily and illustratively as compounds which modify gastrointestinal motility, useful within the meaning of this invention, but without restricting this invention thereto.

As exemplary and illustrative compounds, which reduce the incidence of transient lower esophageal sphincter relaxation, useful within the meaning of this invention GABA-B receptor agonists may be mentioned, such as e.g. each and every compound listed exemplarily expressis verbis in list 23b of this invention, as well as the pharmaceutically acceptable salts, solvates or stereoisomers thereof, without restricting the present invention thereto.

In the context of this invention, as exemplary and illustrative GABA-B receptor agonist BACLOFEN may be alternatively mentioned, but however without restricting this invention thereto in any way.

A notable embodiment of this invention refers to those combinations comprising either as first active agent or as second active agent compounds mentioned exemplarily as being useful in the meaning of this invention; and a further notable embodiment of this invention refers to those combinations comprising both as first active agent and as second active agent compounds mentioned exemplarily as being useful in the meaning of this invention.

Biological Investigations

Measurement of Gastric Venting in the Dog

The effect of a combination of certain acid pump antagonists and compounds which reduce the incidence of transient lower esophageal sphincter relaxation (TLOSR) can be studied as follows:

The technique has been developed to quantify the number of transient lower esophageal sphincter relaxations (TLOSRs, leading to eructations) in the conscious dog. The technique can be used with fasted or fed animals and it is not depending on the status of gastric acid secretion.

For the assessment of TLOSRs, gastric fistula dogs are temporarily connected via the gastric fistula to a special barostat that continuously measures the gastric pressure and continuously approximates a target pressure by pumping or sucking the gas mixture, containing 1-2% hydrogen.

A level of target pressure is selected that causes an appropriate number of TLOSRs, usually for a period of 30 min. Appropriate means that there has to be a sufficiently high number of TLOSRs to enable estimation of a compound-induced reduction of the number of TLOSRs, but, on the other hand, not too many, since the registration technique has a resolution of about 1 eructation/minute.

The quantification of eructations is performed by continuous collection of the air in front of and in the middle of nose and mouth. If the dog is belching, the air, aerated by hydrogen (coming from the gastric gas mixture) is sucked to a hydrogen sensor registering hydrogen concentration. Enhancement by a distinct extent in hydrogen concentration in the collected air is defined to represent an eructation. No eructations are usually caused by swallows nor do eructations occur without elevated gastric pressure. The threshold for the induction of eructation has been found to be about 10 mm Hg.

The effect of a placebo and of certain acid pump antagonists or compounds which reduce the incidence of transient lower esophageal, as well as, in particular, the effect of a these both in combination on the number of transient lower esophageal sphincter relaxations, can be measured under appropriate conditions.

The results obtained in this newly developed test system clearly demonstrate the potential of this in vivo model with respect to an easy, fast and reliable assessment of TLOSRs inhibiting compounds. The applicability of this model is not restricted to a specific mode of action of a compound, therefore being of great value in the identification of compounds with a new mechanism of action.

The technique seems to be superior over other techniques for easy, fast and convenient measurement of TLOSRs. Thus, esophageal pH-metry depends on availability of gastric acid for the registration of gastro-esophageal reflux events. The applicability of the multilumen catheter technique in conscious animals depends on the existence of an esophagostomy to enter the esophagus, to penetrate the lower esophageal sphincter and to enter the stomach. The technique is therefore not independent on physiological perturbations in the region of interest. By contrast, our new technique allows for the registration of TLOSRs under conditions of minimal physiological interference of the lower esophageal sphincter as the only impact to the biology is the gastric fistula in the most dependent position of the stomach.

Thus, a further aspect of the present invention relates to a method to measure compound-associated modulation of the number of transient lower esophageal sphincter relaxations (TLOSRs) comprising the following steps

a.) connecting a gastric fistula animal via the gastric fistula to a barostat which continuously adjusts an elevated gastric target pressure by pumping or sucking a suitable gas mixture containing a suitable detecting gas causing an appropriate number of TLOSRs leading to eructations,

b.) administering one or more of said compounds optionally sequentially, separately or simultaneously to said animal,

c.) quantificating said TLOSRs via measuring the numbers of said eructations by detecting quantitatively the concentration of detecting gas eructated.

In this context, it is to be stressed, that said gastric fistula animal is suitably a gastric fistula dog, although other current animals may work as well.

Yet it is to be stressed, that said detecting gas is suitably mixed with air, although other gases, such as nitrogen, may work as well.

Further it is to be stressed, that said detecting (i.e. marker) gas is suitably hydrogen, although other gases, such as SF₆, may work as well.

Yet further it is to be stressed, that said gas mixture is suitably air containing 1-2% hydrogen, although higher concentrations may work as well, in particular until the maximum undangerous concentration of 3.6% hydrogen.

Yet in this context, it is to be stressed, that, when said animal is a dog, said gastric target pressure is suitably 10 mm Hg. But depending on the dog breed and on the individual properties, other intragastric presssures may work as well.

IBS Models

The effect of a combination of certain acid pump antagonists and compounds which modify gastrointestinal motility regarding the therapy of irritable bowel syndrome (IBS) can be studied in art-known test systems, such as e.g. one of those described in E. A. Mayer and S. M. Collins, Gastroenterology 122, p. 2032-2048 (2002), or in a model analogous or similar thereto.

1. A combination comprising a first active ingredient, which is at least one acid pump antagonist being a tricyclic imidazopyridine compound selected from the group consisting of
(7S,8R,9R)-2,3-dimethyl-7,8-dihydroxy-9-phenyl-7,8,9,10-tetrahydroimidazo[1,2-h][1,7]naphthyridine,
(7S,8R,9R)-7,8-isopropylidenedioxy-2,3-dimethyl-9-phenyl-7,8,9,10-tetrahydroimidazo[1,2-h][1,7]naphthyridine,
7,8-dihydroxy-9-phenyl-2,3-dimethyl-7H-8,9-dihydropyrano[2,3-c]imidazo[1,2-a]pyridine,
(7R,8R,9R)-2,3-dimethyl-8-hydroxy-7-methoxy-9-phenyl-7,8,9,10-tetrahydroimidazo[1,2-h][1,7]naphthyridine,
(7S,8S,9S)-2,3-dimethyl-8-hydroxy-7-methoxy-9-phenyl-7,8,9,10-tetrahydroimidazo[1,2-h][1,7]naphthyridine,
(7S,8R,9R)-2,3-dimethyl-8-hydroxy-7-methoxy-9-phenyl-7,8,9,10-tetrahydroimidazo[1,2-h][1,7]naphthyridine,
(7R,8S,9S)-2,3-dimethyl-8-hydroxy-7-methoxy-9-phenyl-7,8,9,10-tetrahydroimidazo[1,2-h][1,7]naphthyridine,
(7R,8R,9R)-2,3-dimethyl-7-ethoxy-8-hydroxy-9-phenyl-7,8,9,10-tetrahydroimidazo[1,2-h][1,7]naphthyridine,
(7S,8R,9R)-2,3-dimethyl-7-ethoxy-8-hydroxy-9-phenyl-7,8,9,10-tetrahydroimidazo[1,2-h][1,7]naphthyridine,
(7R,8R,9R)-2,3-dimethyl-8-hydroxy-7-(2-methoxyethoxy)-9-phenyl-7,8,9,10-tetrahydroimidazo[1,2-h][1,7]naphthyridine,
(7S,8S,9S)-2,3-dimethyl-8-hydroxy-7-(2-methoxyethoxy)-9-phenyl-7,8,9,10-tetrahydroimidazo[1,2-h][1,7]naphthyridine,
(7S,8R,9R)-2,3-dimethyl-8-hydroxy-7-(2-methoxyethoxy)-9-phenyl-7,8,9,10-tetrahydroimidazo[1,2-h][1,7]naphthyridine,
(7R,8S,9S)-2,3-dimethyl-8-hydroxy-7-(2-methoxyethoxy)-9-phenyl-7,8,9,10-tetrahydroimidazo[1,2-h][1,7]naphthyridine,
(7S,8R,9R)-2,3-dimethyl-8-hydroxy-9-phenyl-7-(2-propoxy)-7,8,9,10-tetrahydroimidazo[1,2-h][1,7]naphthyridine,
(7R,8R,9R)-2,3-dimethyl-7,8-dimethoxy-9-phenyl-7,8,9,10-tetrahydroimidazo[1,2-h][1,7]naphthyridine,
(7R,8R,9R)-2,3-dimethyl-8-hydroxy-7-(2-methylthioethyloxy)-9-phenyl-7,8,9,10-tetrahydroimidazo[1,2-h][1,7]-naphthyridine,
(7S,8R,9R)-2,3-dimethyl-8-hydroxy-7-(2-methylthioethyloxy)-9-phenyl-7,8,9,10-tetrahydroimidazo[1,2-h][1,7]-naphthyridine,
(7R,8R,9R)-2,3-dimethyl-8-hydroxy-7-(2-methylsulphinylethoxy)-9-phenyl-7,8,9,10-tetrahydroimidazo-[1,2-h][1,7]naphthyridine,
(7S,8R,9R)-2,3-dimethyl-8-hydroxy-7-(2-methylsulphinylethoxy)-9-phenyl-7,8,9,10-tetrahydroimidazo-[1,2-h][1,7]naphthyridine,
(7R,8R,9R)-2,3-dimethyl-8-hydroxy-7-(ethylthio)-9-phenyl-7,8,9,10-tetrahydroimidazo[1,2-h][1,7]naphthyridine,
(7S,8R,9R)-2,3-dimethyl-8-hydroxy-7-(ethylthio)-9-phenyl-7,8,9,10-tetrahydroimidazo[1,2-h][1,7]naphthyridine,
(7R,8R,9R)-2,3-dimethyl-8-hydroxy-7-(2,2,2-trifluoroethoxy)-9-phenyl-7,8,9,10-tetrahydroimidazo-[1,2-h][1,7]naphthyridine,
(7S,8R,9R)-2,3-dimethyl-8-hydroxy-7-(2,2,2-trifluoroethoxy)-9-phenyl-7,8,9,10-tetrahydroimidazo-[1,2-h][1,7]naphthyridine,
(7S,8R,9R)-8-acetoxy-7-(2-methoxyethoxy)-2,3-dimethyl-9-phenyl-7,8,9,10-tetrahydroimidazo[1,2-h][1,7]naphthyridine,
(7R,8R,9R)-8-acetoxy-7-(2-methoxyethoxy)-2,3-dimethyl-9-phenyl-7,8,9,10-tetrahydroimidazo[1,2-h][1,7]naphthyridine,
(7R,8R,9R)-8-acetoxy-7-methoxy-2,3-dimethyl-9-phenyl-7,8,9,10-tetrahydroimidazo[1,2-h][1,7]naphthyridine,
(7R,8R,9R)-8-acetoxy-7-ethoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-9-phenyl-8-propionyloxy-7,8,9,10-tetrahydroimidazo[1,2-h]-[1,7]naphthyridine,
(7R,8R,9R)-8-benzoyloxy-7-(2-methoxyethoxy)-2,3-dimethyl-9-phenyl-7,8,9,10-tetrahydroimidazo[1,2-h][1,7]naphthyridine,
(7S,8R,9R)-8-benzoyloxy-7-(2-methoxyethoxy)-2,3-dimethyl-9-phenyl-7,8,9,10-tetrahydroimidazo[1,2-h][1,7]naphthyridine,
(7R,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7S,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-7-methoxy-2,3-dimethyl-9-phenyl-7,8,9,10-tetrahydroimidazo[1,2-h][1,7]naphthyridine,
(7S,8R,9R)-8-benzoyloxy-7-methoxy-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(4-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(3-nitrobenzoyloxy)-9-phenyl-7,8,9,10-tetrahydroimidazo[1,2-h][1,7]naphthyridine,
(7S,8R,9R)-7-methoxy-2,3-dimethyl-8-(3-nitrobenzoyloxy)-9-phenyl-7,8,9,10-tetrahydroimidazo[1,2-h][1,7]naphthyridine,
(7R,8R,9R)-7-methoxy-2,3-dimethyl-8-(3-nitrobenzoyloxy)-9-phenyl-7,8,9,10-tetrahydroimidazo[1,2-h][1,7]naphthyridine,
(7S,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(4-methoxybenzoyloxy)-9-phenyl-7,8,9,10-tetrahydroimidazo[1,2-h][1,7]naphthyridine,
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7S,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-7-(2-methoxyethoxy)-8-(N,N-diethylaminocarbonyloxy)-2,3-dimethyl-9-phenyl-7,8,9,10-tetrahydroimidazo[1,2-h][1,7]naphthyridine,
(7R,8R,9R)-8-ethylaminocarbonyloxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-2,3-dimethyl-7-(2-methoxyethoxy)-9-phenyl-7H-8,9-dihydropyrano[2,3-c]imidazo[1,2-a]pyridine,
(7S,8R,9R)-8-benzoyloxy-2,3-dimethyl-7-(2-methoxyethoxy)-9-phenyl-7H-8,9-dihydropyrano[2,3-c]imidazo[1,2-a]pyridine,
(7R,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-8-[4-(methoxycarbonyl)-benzoyloxy]-2,3-dimethyl-7-(2-methoxyethoxy)-9-phenyl-7H-8,9-dihydropyrano[2,3-c]imidazo[1,2-a]pyridine,
(7S,8R,9R)-2,3-dimethyl-7-methoxy-8-methoxyacetyloxy-9-phenyl-7,8,9,10-tetrahydroimidazo[1,2-h][1,7]naphthyridine,
(7R,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,
(7S,8R,9R)-8-(N,N-diethylaminocarbonyloxy)-2,3-dimethyl-7-methoxy-9-phenyl-7,8,9,10-tetrahydroimidazo[1,2-h][1,7]naphthyridine,
(7R,8R,9R)-7-methoxy-8-methoxycarbonyloxy-2,3-dimethyl-9-phenyl-7,8,9,10-tetrahydroimidazo[1,2-h][1,7]naphthyridine,
(7S,8R,9R)-7-methoxy-8-methoxycarbonyloxy-2,3-dimethyl-9-phenyl-7,8,9,10-tetrahydroimidazo[1,2-h][1,7]naphthyridine,
(7R,8R,9R)-2,3-dimethyl-8-formyloxy-7-methoxy-9-phenyl-7,8,9,10-tetrahydroimidazo[1,2-h][1,7]naphthyridine,
(7S,8R,9R)-2,3-dimethyl-8-formyloxy-7-methoxy-9-phenyl-7,8,9,10-tetrahydroimidazo[1,2-h][1,7]naphthyridine,
(7R,8R,9R)-8-benzoyloxy-2,3-dimethyl-7-methoxy-9-phenyl-7,8,9,10-tetrahydroimidazo[1,2-h][1,7]naphthyridine,
(7R,8S,9R)-2,3,8-trimethyl-7,8-dihydroxy-9-phenyl-7,8,9,10-tetrahydroimidazo[1,2-h][1,7]naphthyridine,
(7S,8S,9R)-2,3-dimethyl-8-benzyl-7,8-dihydroxy-9-phenyl-7,8,9,10-tetrahydroimidazo[1,2-h][1,7]naphthyridine,
(7R,8S,9R)-2,3,8-trimethyl-7,8-0,0-isopropylidene-9-phenyl-7,8,9,10-tetrahydroimidazo[1,2-h][1,7]naphthyridine,
(7S,8S,9R)-2,3,8-trimethyl-7-(2-methoxyethoxy)-8-hydroxy-9-phenyl-7,8,9,10-tetrahydroimidazo[1,2-h][1,7]naphthyridine,
(7S,8S,9R)-2,3,8-trimethyl-7-methoxy-8-hydroxy-9-phenyl-7,8,9,10-tetrahydroimidazo[1,2-h][1,7]naphthyridine,
(7R,8R,9R)-2,3,7-trimethyl-7,8-dihydroxy-9-phenyl-7,8,9,10-tetrahydroimidazo[1,2-h][1,7]naphthyridine,
(7R,8R,9R)-2,3,7-trimethyl-7,8-[1,3]dioxolo-9-phenyl-7,8,9,10-tetrahydroimidazo[1,2-h][1,7]naphthyridine,
(8S,9R)-2,3-dimethyl-8-hydroxy-7-methylidene-9-phenyl-7,8,9,10-tetrahydroimidazo[1,2-h][1,7]naphthyridine,
(7S,8R,9R)-2,3,7-trimethyl-7,8-dihydroxy-9-phenyl-7H-8,9-dihydropyrano[2,3-c]imidazo[1,2-a]pyridine,
(7R,8R,9R)-2,3,7-trimethyl-7,8-dihydroxy-9-phenyl-7H-8,9-dihydropyrano[2,3-c]imidazo[1,2-a]pyridine,
(7S,8R,9R)-2,3-dimethyl-7,8-dihydroxy-7,9-diphenyl-7H-8,9-dihydropyrano[2,3-c]imidazo[1,2-a]pyridine,
(7S,8R,9R)-2,3-dimethyl-7-(2′,2′-dimethylvinyl)-7,8-dihydroxy-9-phenyl-7H-8,9-dihydropyrano[2,3-c]imidazo[1,2-a]pyridine,
(7R,8R,9R)-2,3-dimethyl-7,8-O-isopropylidene-9-phenyl-7-vinyl-7H-8,9-dihydropyrano[2,3-c]imidazo[1,2-a]pyridine,
(7R,8R,9R)-2,3-dimethyl-8-hydroxy-7-(2-methoxyethoxy)-9-phenyl-7H-8,9-dihydropyrano[2,3-c]imidazo[1,2-a]pyridine,
(7S,8R,9R)-2,3-dimethyl-8-hydroxy-7-(2-methoxyethoxy)-9-phenyl-7H-8,9-dihydropyrano[2,3-c]imidazo[1,2-a]pyridine,
(7R,8R,9R)-2,3-dimethyl-8-hydroxy-7-ethoxy-9-phenyl-7H-8,9-dihydropyrano[2,3-c]imidazo[1,2-a]pyridine,
(7S,8R,9R)-2,3-dimethyl-8-hydroxy-7-ethoxy-9-phenyl-7H-8,9-dihydropyrano[2,3-c]imidazo[1,2-a]pyridine,
(7R,8R,9R)-2,3-dimethyl-8-hydroxy-7-(2-methoxypropoxy)-9-phenyl-7H-8,9-dihydropyrano[2,3-c]imidazo[1,2-a]pyridine,
(7S,8R,9R)-2,3-dimethyl-8-hydroxy-7-(2-methoxypropoxy)-9-phenyl-7H-8,9-dihydropyrano[2,3-c]imidazo[1,2-a]pyridine,
(7R,8R,9R)-2,3-dimethyl-8-hydroxy-7-(2-propoxy)-9-phenyl-7H-8,9-dihydropyrano[2,3-c]imidazo[1,2-a]pyridine,
(7S,8R,9R)-2,3-dimethyl-8-hydroxy-7-(2-propoxy)-9-phenyl-7H-8,9-dihydropyrano[2,3-c]imidazo[1,2-a]pyridine,
(7R,8R,9R)-2,3-dimethyl-8-hydroxy-7-butoxy-9-phenyl-7H-8,9-dihydropyrano[2,3-c]imidazo[1,2-a]pyridine,
(7S,8R,9R)-2,3-dimethyl-8-hydroxy-7-butoxy-9-phenyl-7H-8,9-dihydropyrano[2,3-c]imidazo[1,2-a]pyridine,
(7S,8R,9R)-7,8-dihydroxy-6-methoxymethyl-2,3-dimethyl-9-phenyl-7,8,9,10-tetrahydroimidazo[1,2-h][1,7]naphthyridine,
(7R,8R,9R)-7,8-dihydroxy-6-methoxymethyl-2,3-dimethyl-9-phenyl-7,8,9,10-tetrahydroimidazo[1,2-h][1,7]naphthyridine,
(7S,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-methoxy-6-methoxymethyl-2,3-dimethyl-9-phenyl-7,8,9,10-tetrahydroimidazo[1,2-h][1,7]naphthyridine,
(7R,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7S,8R,9R)-8-hydroxy-7-(2-methoxyethoxy)-6-methoxymethyl-2,3-dimethyl-9-phenyl-7,8,9,10-tetrahydroimidazo[1,2-h][1,7]naphthyridine,
(7R,8R,9R)-8-hydroxy-7-ethoxy-6-methoxymethyl-2,3-dimethyl-9-phenyl-7,8,9,10-tetrahydro-imidazo[1,2-h][1,7]naphthyridine,
(7S,8R,9R)-8-hydroxy-7-ethoxy-6-methoxymethyl-2,3-dimethyl-9-phenyl-7,8,9,10-tetrahydro-imidazo[1,2-h]-[1,7]naphthyridine,
7,8-dihydroxy-2,3-dimethyl-9-(3-thienyl)-7,8,9,10-tetrahydroimidazo[1,2-h][1,7]naphthyridine,
7-hydroxy-2,3-dimethyl-9-(3-thienyl)-7,8,9,10-tetrahydroimidazo[1,2-h][1,7]naphthyridine,
9-(3-furyl)-7-hydroxy-2,3-dimethyl-7,8,9,10-tetrahydroimidazo[1,2-h][1,7]naphthyridine,
(7R,8R,9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7S,8R 9R)-8-hydroxy-7-[2-(2-methoxyethoxy)ethoxy]-2,3-dimethyl-9-phenyl-7,8,9,10-tetrahydroimidazo[1,2-h][1,7]naphthyridine,
(7R,8R,9R)-7,8-dihydroxy-2-methyl-9-phenyl-7,8,9,10-tetrahydroimidazo[1,2-h][1,7]naphthyridine,
(7S,8R,9R)-8-hydroxy-2-methyl-7-(2-methoxyethoxy)-9-phenyl-7,8,9,10-tetrahydroimidazo[1,2-h][1,7]naphthyridine,
(7R,8R,9R)-8-hydroxy-2-methyl-7-(2-methoxyethoxy)-9-phenyl-7,8,9,10-tetrahydroimidazo[1,2-h][1,7]naphthyridine,
(7R,8R,9R)-3-bromo-8-hydroxy-7-(2-methoxyethoxy)-2-methyl-9-phenyl-7,8,9,10-tetrahydroimidazo-[1,2-h][1,7]naphthyridine,
(7R,8R,9R)-3-chloro-8-hydroxy-7-(2-methoxyethoxy)-2-methyl-9-phenyl-7,8,9,10-tetrahydroimidazo-[1,2-h][1,7]naphthyridine,
(7R,8R,9R)-3-bromo-7-hydroxy-8-(2-methoxyethoxy)-2-methyl-9-phenyl-7,8,9,10-tetrahydroimidazo-[1,2-h][1,7]naphthyridine,
(7R,8R,9R)-3-chloro-8-hydroxy-7-(2-methoxyethoxy)-2-methyl-9-phenyl-7H-8,9-dihydro-pyrano[2,3-c]imidazo[1,2-a]pyridine,
(7R,8R,9R)-8-hydroxy-7-(2-methoxyethoxy)-2-methyl-9-phenyl-7H-8,9-dihydro-pyrano[2,3-c]imidazo[1,2-a]pyridine,
(7R,8R,9R)-7,8-dihydroxy-2-methyl-9-phenyl-7H-8,9-dihydropyrano[2,3-c]imidazo[1,2-a]pyridine,
(7S,8R,9R)-7,8-dihydroxy-2-methyl-9-phenyl-7,8,9,10-tetrahydroimidazo[1,2-h][1,7]naphthyridine,
(7R,8R,9R)-8-hydroxy-7-methoxy-2-methyl-9-phenyl-7,8,9,10-tetrahydroimidazo[1,2-h][1,7]naphthyridine,
(7S,8R,9R)-8-hydroxy-7-methoxy-2-methyl-9-phenyl-7,8,9,10-tetrahydroimidazo[1,2-h][1,7]naphthyridine,
(7R,8R,9R)-2,3-dimethyl-8-hydroxy-7-(2-hydroxyethoxy)-9-phenyl-7,8,9,10-tetrahydroimidazo[1,2-h][1,7]naphthyridine,
(7R,8R,9R)-3,9-diphenyl-8-hydroxy-7-(2-methoxyethoxy)-2-methyl-7,8,9,10-tetrahydroimidazo[1,2-h][1,7]naphthyridine,
(7R,8R,9R)-7,8-dihydroxy-2-methoxymethyl-3-methyl-9-phenyl-7,8,9,10-tetrahydroimidazo[1,2-h][1,7]naphthyridine,
(7S,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7R,8R,9R)-8-hydroxy-7-(2-methoxyethoxy)-2-methoxymethyl-3-methyl-9-phenyl-7,8,9,10-tetrahydroimidazo[1,2-h][1,7]naphthyridine,
(7S,8R,9R)-7-ethoxy-8-hydroxy-2-methoxymethyl-3-methyl-9-phenyl-7,8,9,10-tetrahydroimidazo[1,2-h][1,7]naphthyridine,
(7R,8R,9R)-7-ethoxy-8-hydroxy-2-methoxymethyl-3-methyl-9-phenyl-7,8,9,10-tetrahydroimidazo[1,2-h][1,7]naphthyridine,
(7R,8S,9R)-10-acetyl-8-hydroxy-2,3-dimethyl-7-(4-morpholino)-7,8,9,10-tetrahydroimidazo[1,2-h][1,7]naphthyridine,
(7R,8S,9R)-8-hydroxy-2,3-dimethyl-7-(4-morpholino)-7,8,9,10-tetrahydroimidazo[1,2-h][1,7]naphthyridine,
(7R,8S,9R)-10-acetyl-8-hydroxy-2,3-dimethyl-7-methylamino-7,8,9,10-tetrahydro-imidazo[1,2-h][1,7]naphthyridine,
(7R,8S,9R)-8-hydroxy-2,3-dimethyl-7-methylamino-7,8,9,10-tetrahydroimidazo[1,2-h]L[1,7]naphthyridine,
(7R,8S,9R)-10-acetyl-8-hydroxy-2,3-dimethyl-7-(1-pyrrolidino)-7,8,9,10-tetrahydroimidazo[1,2-h][1,7]naphthyridine,
(7R,8S,9R)-8-hydroxy-2,3-dimethyl-7-(1-pyrrolidino)-7,8,9,10-tetrahydroimidazo[1,2-h][1,7]naphthyridine,
(7R,8S,9R)-10-acetyl-7-benzylamino-8-hydroxy-2,3-dimethyl-7,8,9,10-tetrahydro-imidazo[1,2-h][1,7]naphthyridine,
(7R,8S,9R)-7-benzylamino-8-hydroxy-2,3-dimethyl-7,8,9,10-tetrahydroimidazo[1,2-h][1,7]naphthyridine,
(7R,8S,9R)-10-acetyl-8-hydroxy-7-(2-methoxyethylamino)-2,3-dimethyl-7,8,9,10-tetrahydro-imidazo[1,2-h][1,7]naphthyridine,
(7R,8S,9R)-8-hydroxy-7-(2-methoxyethylamino)-2,3-dimethyl-7,8,9,10-tetrahydroimidazo[1,2-h][1,7]naphthyridine,
(7R,8S,9R)-10-acetyl-7-(dimethylamino)-8-hydroxy-2,3-dimethyl-7,8,9,10-tetrahydro-imidazo[1,2-h][1,7]naphthyridine,
(7R,8S,9R)-8-hydroxy-7-(dimethylamino)-2,3-dimethyl-7,8,9,10-tetrahydroimidazo[1,2-h][1,7]naphthyridine,
(7S,8S,9R)-8-hydroxy-2,3,7-trimethyl-7,8,9,10-tetrahydroimidazo[1,2-h][1,7]naphthyridine,
(7S,8S,9R)-7-cyanomethyl-8-hydroxy-2,3-dimethyl-7,8,9,10-tetrahydroimidazo[1,2h][1,7]naphthyridine,
(7S,8S,9R)-8-hydroxy-2,3-dimethyl-7-propyl-7,8,9,10-tetrahydroimidazo[1,2-h][1,7]naphthyridine,
(7R,8S,9R)-8-hydroxy-2,3-dimethyl-7-(3-methoxypropyl)-7,8,9,10-tetrahydroimidazo-[1,2-h][1,7]naphthyridine,
2,3-dimethyl-9-phenyl-7H-8,9-dihydro-pyrano-[2,3-c]-N-(diethyl)imidazo[1,2-a]pyridine-6-carboxamide,
ethyl 2,3-dimethyl-9-phenyl-7H-8,9-dihydro-pyrano[2,3-c]-imidazo[1,2-a]pyridine-6-carboxylate,
2,3-dimethyl-9-phenyl-7H-8,9-dihydro-pyrano[2,3-c]-imidazo[1,2-a]pyridine-6-(N,N-dimethyl)carbamide,
(7R,8R,9R)-2,3-dimethyl-7-(2-methoxyethoxy)-9-phenyl-8-(5-nitrooxy-valeryloxy)-7,8,9,10-tetrahydro-imidazo[1,2-h][1,7]naphthyridine,
(7R,8R,9R)-2,3-dimethyl-7-(2-methoxyethoxy)-9-phenyl-8-(4-nitrooxy-butyryloxy)-7,8,9,10-tetrahydro-imidazo[1,2-h][1,7]naphthyridine,
(7R,8R,9R)-2,3-dimethyl-7-(2-methoxyethoxy)-9-phenyl-8-(5-nitro-oxy-valeryloxy)-7H-8,9-dihydro-pyrano[2,3-c]imdazo[1,2-a]pyridine,
(7R,8R,9R)-2,3-dimethyl-7-(2-methoxyethoxy)-9-phenyl-8-(6-nitro-oxy-2-oxa-capryloxy)-7,8,9,10-tetrahydro-imidazo[1,2-h][1,7]naphthyridine,
(7R,8R,9R)-2,3-dimethyl-7-(2-methoxyethoxy)-9-phenyl-8-(4-nitro-oxymethyl-benzoyloxy)-7,8,9,10-tetrahydroimidazo[1,2-h][1,7]naphthyridine,
and the hydrates, solvates, salts, hydrates of the salts and solvates of the salts thereof
and a second active ingredient, which is at least one compound which modifies gastrointestinal motility, selected from the group consisting of a 5-HT-(partial-)agonist/antagonist, a muscarinic antagonist, a kappa opioid receptor agonist, a delta opioid receptor agonist, an opioid receptor agonist, a dopamine receptor antagonist, a cholecystokinin A antagonist, a cholecystokinin B antagonist, an alpha-2 adrenoceptor agonist, a N-methyl-D-aspartate receptor antagonist, a non-N-methyl-D-aspartate glutamate receptor antagonist, a nitric oxide synthase inhibitor, a motilin agonist, a somatostatin agonist/antagonist, a neurotensin agonist/antagonist, a vasoactive intestinal peptide antagonist, a substance P antagonist, a neurokinin antagonist, a calcium channel blocker, a potassium channel opener, a selective serotonin reuptake inhibitor, a corticotropin releasing factor antagonist, a GABA-A receptor agonist, a GABA-B receptor agonist/partial agonist, a gastroprokinetic, an antiemetic and an antispasmodic.

2. The combination according to claim 1 comprising a first active ingredient, which is an acid pump antagonist selected from the group consisting of
(7R,8R,9R)-8-hydroxy-7-(2-methoxyethoxy)-2,3-dimethyl-9-phenyl-7,8,9,10-tetrahydro-imidazo[1,2-h][1,7]naphthyridine,
(7R,8R,9R)-2,3-dimethyl-8-hydroxy-7-methoxy-9-phenyl-7,8,9,10-tetrahydroimidazo[1,2-h][1,7]naphthyridine,
(7S,8R,9R)-2,3-dimethyl-8-hydroxy-7-methoxy-9-phenyl-7,8,9,10-tetrahydroimidazo[1,2-h][1,7]naphthyridine,
(7R,8R,9R)-2,3-dimethyl-7-ethoxy-8-hydroxy-9-phenyl-7,8,9,10-tetrahydroimidazo[1,2-h][1,7]naphthyridine,
(7S,8R,9R)-2,3-dimethyl-7-ethoxy-8-hydroxy-9-phenyl-7,8,9,10-tetrahydroimidazo[1,2-h][1,7]naphthyridine,
(7R,8R,9R)-2,3-dimethyl-8-hydroxy-7-(2-methoxyethoxy)-9-phenyl-7,8,9,10-tetrahydroimidazo[1,2-h][1,7]naphthyridine,
(7R,8R,9R)-8-acetoxy-7-(2-methoxyethoxy)-2,3-dimethyl-9-phenyl-7,8,9,10-tetrahydroimidazo[1,2-h][1,7]naphthyridine,
(7R,8R,9R)-8-benzoyloxy-7-(2-methoxyethoxy)-2,3-dimethyl-9-phenyl-7,8,9,10-tetrahydroimidazo[1,2-h][1,7]naphthyridine,
(7R,8R,9R)-8-methoxycarbonyloxy-7-(2-methoxyethoxy)-2,3-dimethyl-9-phenyl-7,8,9,10-tetrahydroimidazo[1,2-h][1,7]naphthyridine,
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
(7R,8S,9R)-2,3,8-trimethyl-7,8-dihydroxy-9-phenyl-7,8,9,10-tetrahydroimidazo[1,2-h][1,7]naphthyridine,
(7R,8R,9R)-2,3,7-trimethyl-7,8-dihydroxy-9-phenyl-7,8,9,10-tetrahydroimidazo[1,2-h][1,7]naphthyridine,
(7R,8R,9R)-2,3-dimethyl-8-hydroxy-7-(2-methoxyethoxy)-9-phenyl-7H-8,9-dihydropyrano[2,3-c]imidazo[1,2-a]pyridine,
(7R,8R,9R)-2,3-dimethyl-8-hydroxy-7-ethoxy-9-phenyl-7H-8,9-dihydropyrano[2,3-c]imidazo[1,2-a]pyridine,
(7R,8R,9R)-8-hydroxy-2-methyl-7-(2-methoxyethoxy)-9-phenyl-7,8,9,10-tetrahydroimidazo[1,2-h][1,7]naphthyridine,
(7R,8R,9R)-3-chloro-8-hydroxy-7-(2-methoxyethoxy)-2-methyl-9-phenyl-7,8,9,10-tetrahydroimidazo[1,2-h][1,7]naphthyridine,
(7R,8R,9R)-3-chloro-8-hydroxy-7-(2-methoxyethoxy)-2-methyl-9-phenyl-7H-8,9-dihydro-pyrano[2,3-c]imidazo[1,2-a]pyridine,
and the hydrates, solvates, salts, hydrates of the salts and solvates of the salts thereof
and a second active ingredient, which is a compound which modifies gastrointestinal motility,
selected from the group consisting of 5-HT4-partial-agonists, 5-HT4-agonists, 5-HT4-antagonists, 5-HT3-antagonists, 5-HT3-agonists, dual 5-HT3-antagonists/5-HT4-agonists, muscarinic M3 antagonists, kappa opioid receptor agonists, delta opioid receptor agonists, dopamine D2 receptor antagonists, cholecystokinin A antagonists, cholecystokinin B antagonists, motilin agonists, NK2 antagonists, NK3 antagonists, GABA-B receptor agonists and gastroprokinetics.

3. The combination according to claim 1, said combination being a composition comprising a first active ingredient, which is an acid pump antagonist selected from the group consisting of
(7R,8R,9R)-2,3-dimethyl-8-hydroxy-7-(2-methoxyethoxy)-9-phenyl-7,8,9,10-tetrahydro-imidazo-[1,2-h][1,7]naphthyridine,
(7R,8R,9R)-2,3-dimethyl-8-hydroxy-7-(2-methoxyethoxy)-9-phenyl-7H-8,9-dihydro-pyrano[2,3-c]imidazo[1,2-a]pyridine
(7R,8R,9R)-7-(2-methoxyethoxy)-2,3-dimethyl-8-(N,N-dimethylaminomethylcarbonyloxy)-9-phenyl-7,8,9,10-tetrahydroimidazo[1,2-h][1,7]naphthyridine,
and the hydrates, solvates, salts, hydrates of the salts and solvates of the salts thereof
and a second active ingredient, which is a compound which modifies gastrointestinal motility, selected from the group consisting of 5-HT-(partial-)agonists/antagonists, muscarinic antagonists, kappa opioid receptor agonists, delta opioid receptor agonists, opioid receptor agonists, dopamine receptor antagonists, cholecystokinin A antagonists, alpha-2 adrenoceptor agonists, N-methyl-D-aspartate receptor antagonists, non-N-methyl-D-aspartate glutamate receptor antagonists, nitric oxide synthase inhibitors, motilin agonists, somatostatin agonists/antagonists, neurotensin agonists/antagonists, vasoactive intestinal peptide antagonists, substance P antagonists, neurokinin antagonists, calcium channel blockers, potassium channel openers, selective serotonin reuptake inhibitors, corticotropin releasing factor antagonists, GABA-A receptor agonists, GABA-B receptor agonists/partial agonists,
and the pharmaceutically acceptable derivatives thereof;

4. The combination according to claim 1, wherein the first active ingredient is (7R,8R,9R)-2,3-dimethyl-8-hydroxy-7-(2-methoxyethoxy)-9-phenyl-7,8,9,10-tetrahydroimidazo-[1,2-h][1,7]naphthyridine, or a hydrate, solvate, salt, hydrate of a salt or solvate of a salt thereof;
and the second active ingredient, which is a compound which modifies gastrointestinal motility, is selected from the group consisting of 5-HT4-partial-agonists, 5-HT4-agonists, 5-HT4-antagonists, 5-HT3-antagonists, 5-HT3-agonists, dual 5-HT3-antagonists/5-HT4-agonists, muscarinic M3 antagonists, kappa opioid receptor agonists, delta opioid receptor agonists, dopamine D2 receptor antagonists, cholecystokinin A antagonists, chlolecystokinin B antagonists, motilin agonists, NK2 antagonists, NK3 antagonists, GABA-B receptor agonists and gastroprokinetics.

5. A combination comprising a first active ingredient which is a bicyclic imidazopyridine compound selected from the group consisting of
2,3-dimethyl-8-(2-ethyl-6-methylbenzylamino)-N-propyl-imidazo[1,2-a]pyridine-6-carboxamide,
8-(2-ethyl-6-methylbenzylamino)-3-hydroxymethyl-2-methylimidazo[1,2-a]pyridine-6-carboxamide,
2,3-dimethyl-8-(2,6-dimethylbenzylamino)-N-hydroxyethyl-imidazo[1,2-a]pyridine-6-carboxamide,
2,3-dimethyl-8-(2-ethyl-6-methylbenzylamino)-imidazo[1,2-a]pyridine-6-carboxamide,
8-(2-ethyl-6-methylbenzylamino)-N,2,3-trimethylimidazo[1,2-a]pyridine-6-carboxamide,
8-(2-ethyl-6-methylbenzylamino)-N,N,2,3-tetramethylimidazo[1,2-a]pyridine-6-carboxamide,
2,3-dimethyl-8-(2,6-dimethylbenzyl-amino)-imidazo[1,2-a]pyridine-6-carboxamide,
N-[2-(dimethylamine)-2-oxoethyl]-8-(2-ethyl-6-methylbenzylamino)-N,2,3-trimethylimidazo[1,2-a]pyridine-6-carboxamide,
2,3-dimethyl-8-(2-ethyl-4-fluoro-6-methylbenzylamino)-imidazo[1,2-a]-pyridine-6-carboxamide mesylate,
2,3-dimethyl-8-(2-methylbenzylamino)-imidazo[1,2-a]pyridine-6-carboxamide,
2,3-dimethyl-8-(2,6-dimethyl-4-fluoro-benzylamino)-imidazo[1,2-a]pyridine-6-carboxamide mesylate,
2,3-dimethyl-8-(2-methyl-6-isopropylbenzylamino)-imidazo[1,2-a]pyridine-6-carboxamide mesylate,
2,3-dimethyl-8-(2,6-diethyl-benzylamino)-imidazo[1,2-a]pyridine-6-carboxamide,
2,3-dimethyl-8-(2-ethylbenzylamino)-imidazo[1,2-a]pyridine-6-carboxamide,
2,3 dimethyl-8-(2-ethyl-6-methyl-benzylamino)-N-hydroxyethyl-imidazo[1,2-a]pyridine-6-carboxamide,
N-(2,3-dihydroxypropyl)-2,3-dimethyl-8-(2-ethyl-6-methylbenzylamino)-[1,2-a]pyridine-6-carboxamide,
2,3 dimethyl-8-(2-ethyl-6-methyl-benzylamino)-N-(2-methoxyethyl)-imidazo[1,2-a]pyridine-6-carboxamide,
2-methyl-8-(2-ethyl-6-methylbenzylamino)-imidazo[1,2-a]pyridine-6-carboxamide,
2,3-dimethyl-8-(2-bromo-6-methylbenzylamino)-imidazo[1,2-a]pyridine-6-carboxamide,
2,3-dimethyl-8-(2-(2-hydroxyethyl)-6-methylbenzylamino)-imidazo[1,2-a]pyridine-6-carboxamide,
8-(2-ethyl-6-methylbenzylamino)-N,N-bis(2-hydroxyethyl)-
2,3-dimethylimidazo[1,2-a]pyridine-6-carboxamide,
8-(2-ethyl-6-methylbenzylamino)-N-(2-hydroxyethyl)-N,2,3-trimethylimidazo[1,2-a]pyridine-6-carboxamide,
2,3-dimethyl-8-(2-ethyl-6-methylbenzyloxy)-imidazo[1,2-a]pyridine-6-carboxamide,
and the pharmaceutically acceptable hydrates, solvates, salts, hydrates of the salts and solvates of the salts thereof;
and a second active ingredient, which is a compound which modifies gastrointestinal motility, selected from the group consisting of 5-HT4-partial-agonists, 5-HT4-agonists, 5-HT4-antagonists, 5-HT3-antagonists, 5-HT3-agonists, dual 5-HT3-antagonists/5-HT4-agonists, muscarinic M3 antagonists, kappa opioid receptor agonists, delta opioid receptor agonists, dopamine D2 receptor antagonists, cholecystokinin A antagonists, cholecystokinin B antagonists, motilin agonists, NK2 antagonists, NK3 antagonists, and gastroprokinetics.

6. The combination according to claim 1, wherein the second active ingredient is selected from the group consisting of a 5-HT-(partial-)agonist/antagonist, a muscarinic antagonist, an opioid receptor agonist, a dopamine receptor antagonist, a cholecystokinin antagonist,
a 5-HT4 antagonist,
a 5-HT3 antagonist,
a 5-HT4 partial agonist,
a 5-HT4 agonist,
a dual 5-HT3 antagonist/5-HT4 agonist,
a cholecystokinin A antagonist,
a NK-2 antagonist,
a NK-3 antagonist,
a kappa opioid receptor agonist,
a delta opioid receptor agonist and a muscarinic M3 antagonist.

7. The combination according to claim 1, wherein the second active ingredient is selected from the group consisting of a 5-HT-(partial-)agonist/antagonist, a motilin agonist, a dopamine receptor antagonist, a cholecystokinin antagonist,
a 5-HT4 partial agonist,
a 5-HT4 antagonist,
a 5-HT4 agonist,
a 5-HT3-agonist,
a motilin receptor agonist,
a cholecystokinin B antagonist,
and a cholecystokinin A antagonist,

8. The combination according to claim 1, wherein the second active ingredient is a gastroprokinetic,
a motilin receptor agonist,
a dopamine D2 receptor antagonist,
a 5-HT-(partial-)agonist/antagonist,
a 5-HT4 partial agonist,
a 5-HT4 agonist,
a muscarinic M3 antagonist,
a kappa opioid receptor agonist,
a dual 5-HT3-antagonist/5-HT4 agonist,
and a cholecystokinin A antagonist,

9. The combination according to claim 1, wherein the second active ingredient is selected from the group consisting of
any 5-HT4-partial-agonist,
any 5-HT4-agonist
any dual 5-HT3-antagonist/5-HT4-agonist
any 5-HT3-antagonist
and any 5-HT4-antagonist

10. The combination according to claim 1, wherein the second active ingredient is a compound selected from the group consisting of:
(3-amino-2-fluoropropyl)phosphinic acid,
(R)-(3-amino-2-fluoropropyl)phosphinic acid,
(S)-(3-amino-2-fluoropropyl)phosphinic acid,
(3-amino-2-fluoro-1-methyl-propyl)phosphinic acid,
(3-amino-2-oxopropyl)phosphinic acid,
(S)-(3-amino-2-hydroxypropyl)phosphinic acid,
(R)-(3-amino-2-hydroxypropyl)phosphinic acid,
(3-amino-1-fluoro-2-hydroxypropyl)phosphinic acid,
(3-amino-2-fluoro-propyl)(methyl)phosphinic acid,
(2R)-(3-amino-2-fluoro-propyl)(methyl)phosphinic acid,
(2S)-(3-amino-2-fluoro-propyl)(methyl)phosphinic acid,
(3-amino-2-fluoro-1-methylpropyl)(methyl)phosphinic acid,
(3-amino-1-fluoropropyl)phosphinic acid,
3-[(4-chlorobenzyl)amino]propyl(methyl)phosphinic acid,
3-[1-({3-[hydroxy(oxido)phosphino]propyl}amino)-ethyl]benzoic acid acid,
(3-amino-2-fluoropropyl)sulphinic acid,
(2S)-(3-amino-2-fluoropropyl)sulphinic acid,
(2R)-(3-amino-2-fluoropropyl)sulphinic acid,
(2S)-(3-amino-2-hydroxypropyl)sulphinic acid,
(2R)-(3-amino-2-hydroxypropyl)sulphinic acid,
(3-amino-2-oxopropyl)sulphinic acid,
and the pharmaceutically acceptable hydrates, solvates, salts and stereoisomers thereof.

11. The combination according to claim 1, wherein the second active ingredient is a compound selected from the group consisting of:
AZD-3355, BACLOFEN, GABAPENTIN, PAZINACLONE, CGP-29030A, CGP-44532, SL-65.1498, SKF-97541,
4-amino-3-phenylbutanoic acid,
4-amino-3-hydroxybutanoic acid,
4-amino-3-(4-chlorophenyl)-3-hydroxyphenylbutanoic acid,
4-amino-3-(thien-2-yl)butanoic acid,
4-amino-3-(5-chlorothien-2-yl)butanoic acid,
4-amino-3-(5-bromothien-2-yl)butanoic acid,
4-amino-3-(5-methylthien-2-yl)butanoic acid,
4-amino-3-(2-imidazolyl)butanoic acid,
4-guanidino-3-(4-chlorophenyl)butanoic acid,
3-amino-2-(4-chlorophenyl)-1-nitropropane,
(3-aminopropyl)phosphonous acid,
(4-aminobut-2-yl)phosphonous acid,
(3-amino-2-methylpropyl)phosphonous acid,
(3-aminobutyl)phosphonous acid,
(3-amino-2-(4-chlorophenyl)propyl)phosphonous acid,
(3-amino-2-(4-chlorophenyl)-2-hydroxypropyl)phosphonous acid,
(3-amino-2-(4-fluorophenyl)propyl)phosphonous acid,
(3-amino-2-phenylpropyl)phosphonous acid,
(3-amino-2-hydroxypropyl)phosphonous acid,
(E)-(3-aminopropen-1-yl)phosphonous acid,
(3-amino-2-cyclohexylpropyl)phosphonous acid,
(3-amino-2-benzylpropyl)phosphonous acid,
[3-amino-2-(4-methylphenyl)propyl]phosphonous acid,
[3-amino-2-(4-trifluoromethylphenyl)propyl]phosphonousacid,
[3-amino-2-(4-methoxyphenyl)propyl]phosphonous acid,
[3-amino-2-(4-chlorophenyl)-2-hydroxypropyl]phosphonousacid,
(3-aminopropyl)methylphosphinic acid,
(3-amino-2-hydroxypropyl)methylphosphinic acid,
(3-aminopropyl)(difluoromethyl)phosphinic acid,
(4-aminobut-2-yl)methylphosphinic acid,
(3-amino-1-hydroxypropyl)methylphosphinic acid,
(3-amino-2-hydroxypropyl)(difluoromethyl)phosphinic acid,
(E)-(3-aminopropen-1-yl)methylphosphinic acid,
(3-amino-2-oxo-propyl)methyl phosphinic acid,
(3-aminopropyl)hydroxymethylphosphinic acid,
(5-aminopent-3-yl)methylphosphinic acid,
(4-amino-1,1,1-trifluorobut-2-yl)methylphosphinic acid,
(3-amino-2-(4-chlorophenyl)propyl)sulfinic acid,
3-aminopropylsulfinic acid
and the pharmaceutically acceptable hydrates, solvates, salts and stereoisomers thereof.

12. The combination according to claim 1, wherein the second active ingredient is a compound selected from the group consisting of
TEGASEROD
MOSAPRIDE,
PRUCALOPRIDE,
BIMU1, ITASETRON, CISAPRIDE, NOR-CISAPRIDE, (+)-NOR-CISAPRIDE, RENZAPRIDE, ZACOPRIDE, SB 205149, SC 53116, RS 67333, RS 67506, (S)-RS 56532, LINTOPRIDE, FABESETRON, E-3620,
BENESETRON, ZATOSETRON, EM-523, DAZOPRIDE, BATANOPRIDE, AS-5370, MCL-225, WAY-100289, YM-114, CILANSETRON, LERISETRON, MIRESETRON, RS-25259-197, T-82, INDISETRON, RS-42358-197, DOLASETRON, PALONOSETRON, AZASETRON, TROPISETRON, ONDANSETRON, GRANISETRON, ALOSETRON, RAMOSETRON, INDISETRON,
PIBOSEROD, LY-353433,
YM-31636, and
PUMOSETRAG.

13. A combination according to claim 1, wherein the second active ingredient is a compound which modifies gastrointestinal motility, selected from the group consisting of
(S)-OXYBUTININ, ALEMCINAL, ALIZAPRIDE, ALOSETRON, ALTINICLINE, ALVIMOPAN, APREPITANT, AZASETRON, BATANOPRIDE, BROMOPRIDE, CILANSETRON, CINITAPRIDE, CISAPRIDE, CLEBOPRIDE, DARIFENACIN, DAZOPRIDE, DEXANABINOL, DEXLOXIGLUMIDE, DIFENIDOL, DOBUPRIDE, DOMPERIDONE, E-3620, EXEPANOL, FABESETRON, FEDOTOZINE, GRANISETRON, INDISETRON, ITASETRON, ITOPRIDE, KW-5092, KW-5139, LERISETRON, LEVOSULPIRIDE, LINTOPRIDE, LIREXAPRIDE, LY-353433, METOCLOPRAMIDE, MITEMCINAL, MOSAPRIDE, ONDANSETRON, PALONOSETRON, PIBOSEROD, PRUCALOPRIDE, R-137696, RAMOSETRON, RENZAPRIDE, RS-25259-197, SR-58611-A, TEGASEROD, TIAPRIDE, TICALOPRIDE, TRIMEBUTINE, TROPISETRON, VOFOPITANT, Z-338, ZACOPRIDE,
and the pharmacologically acceptable derivatives thereof.

14. A combination according to claim 1, wherein the second active ingredient is a compound which modifies gastrointestinal motility, selected from the group consisting of
ALEMCINAL, ASIMADOLINE, BACLOFEN, BIPERIDEN, CILANSETRON, CINITAPRIDE, CISAPRIDE, CLEBOPRIDE, DARIFENACIN, DAZOPRIDE, DIFENIDOL, DOBUPRIDE, E-3620, EM-523, FABESETRON, FEDOTOZINE, GABAPENTIN, IDREMCINAL, ITOPRIDE, KW-5092, KW-5139, LEVOSULPIRIDE, LINTOPRIDE, LIREXAPRIDE, MEBEVERINE, METOCLOPRAMIDE, MITEMCINAL, MOSAPRIDE, NITRAQUAZONE, PAZINACLONE, PIBOSEROD, PRIDINOL, PROCYCLIDINE, PRUCALOPRIDE, PUMOSETRAG, R-137696, RENZAPRIDE, RICASETRON, ROLIPRAM, SK-896, SL-65.1498, SR-58611-A, T-1815, TEGASEROD, TIBENELAST, TICALOPRIDE, TRIHEXYPHENIDYL, Y-36912, YM-114, YM-47813, Z-338 and ZACOPRIDE,
and the pharmacologically acceptable derivatives thereof,

15. A combination according to claim 1, wherein the second active ingredient is TEGASEROD or a salt or a tautomer thereof.

16. A combination according to claim 1, wherein said combination is a pharmaceutical composition comprising the first and second active ingredient in admixture for simultaneous oral administration, and further comprising one or more pharmaceutically acceptable carriers, diluents, adjuvants, auxiliaries and/or excipients.

17. A combination according to claim 1, wherein said combination is a combined preparation, for simultaneous, sequential, separate or chronologically staggered administration.

18. A combination according to claim 1, wherein said combination is a fixed combination comprising the first and second active ingredient together in one unit dosage or in the form of a single entity.

19. A commercial package comprising at least one acid pump antagonist as defined in claim 1, as active ingredient together with instruction for simultaneous, sequential, separate or chronologically staggered use with at least one compound, which modifies gastrointestinal motility, as defined in claim 1.

20. A commercial package comprising at least one compound, which modifies gastrointestinal motility, as defined in claim 1 as active ingredient together with instruction for simultaneous, sequential, separate or chronologically staggered use with at least one acid pump antagonist as defined in claim 1.

21.-23. (canceled)

24. A method of normalizing, stabilizing or regulating altered gastrointestinal motility, sensitivity and/or secretion in a patient comprising administering simultaneously, separately or sequentially a therapeutically effective and tolerable amount of an acid pump antagonist as defined in claim 1; and a therapeutically effective and tolerable amount of a compound which modifies gastrointestinal motility, as defined in claim 1 to a patient in need thereof.

25. A method of reducing the incidence of transient lower esophageal sphincter relaxation (TLOSR) in a patient comprising administering simultaneously, separately or sequentially a therapeutically effective and tolerable amount of an acid pump antagonist as defined in claim 1; and a therapeutically effective and tolerable amount of a compound which modifies gastrointestinal motility, to a patient in need thereof,
wherein said compound which modifies gastrointestinal motility is selected from the group consisting of
(3-amino-2-fluoropropyl)phosphinic acid,
(R)-(3-amino-2-fluoropropyl)phosphinic acid,
(S)-(3-amino-2-fluoropropyl)phosphinic acid,
(3-amino-2-fluoro-1-methyl-propyl)phosphinic acid,
(3-amino-2-oxopropyl)phosphinic acid,
(S)-(3-amino-2-hydroxypropyl)phosphinic acid,
(R)-(3-amino-2-hydroxypropyl)phosphinic acid,
(3-amino-1-fluoro-2-hydroxypropyl)phosphinic acid,
(3-amino-2-fluoro-propyl)(methyl)phosphinic acid,
(2R)-(3-amino-2-fluoro-propyl)(methyl)phosphinic acid,
(2S)-(3-amino-2-fluoro-propyl)(methyl)phosphinic acid,
(3-amino-2-fluoro-1-methylpropyl)(methyl)phosphinic acid,
(3-amino-1-fluoropropyl)phosphinic acid,
3-[(4-chlorobenzyl)amino]propyl(methyl)phosphinic acid,
3-[1-({3-[hydroxy(oxido)phosphino]propyl}amino)-ethyl]benzoic acid acid,
(3-amino-2-fluoropropyl)sulphinic acid,
(2S)-(3-amino-2-fluoropropyl)sulphinic acid,
(2R)-(3-amino-2-fluoropropyl)sulphinic acid,
(2S)-(3-amino-2-hydroxypropyl)sulphinic acid,
(2R)-(3-amino-2-hydroxypropyl)sulphinic acid,
(3-amino-2-oxopropyl)sulphinic acid,
AZD-3355, Baclofen, Gabapentin, Pazinaclone, CGP-29030A, CGP-44532, SL-65.1498, SKF-97541,
4-amino-3-phenylbutanoic acid,
4-amino-3-hydroxybutanoic acid,
4-amino-3-(4-chlorophenyl)-3-hydroxyphenylbutanoic acid,
4-amino-3-(thien-2-yl)butanoic acid,
4-amino-3-(5-chlorothien-2-yl)butanoic acid,
4-amino-3-(5-bromothien-2-yl)butanoic acid,
4-amino-3-(5-methylthien-2-yl)butanoic acid,
4-amino-3-(2-imidazolyl)butanoic acid,
4-guanidino-3-(4-chlorophenyl)butanoic acid,
3-amino-2-(4-chlorophenyl)-1-nitropropane,
(3-aminopropyl)phosphonous acid,
(4-aminobut-2-yl)phosphonous acid,
(3-amino-2-methylpropyl)phosphonous acid,
(3-aminobutyl)phosphonous acid,
(3-amino-2-(4-chlorophenyl)propyl)phosphonous acid,
(3-amino-2-(4-chlorophenyl)-2-hydroxypropyl)phosphonous acid,
(3-amino-2-(4-fluorophenyl)propyl)phosphonous acid,
(3-amino-2-phenylpropyl)phosphonous acid,
(3-amino-2-hydroxypropyl)phosphonous acid,
(E)-(3-aminopropen-1-yl)phosphonous acid,
(3-amino-2-cyclohexylpropyl)phosphonous acid,
(3-amino-2-benzylpropyl)phosphonous acid,
[3-amino-2-(4-methylphenyl)propyl]phosphonous acid,
[3-amino-2-(4-trifluoromethylphenyl)propyl]phosphonousacid,
[3-amino-2-(4-methoxyphenyl)propyl]phosphonous acid,
[3-amino-2-(4-chlorophenyl)-2-hydroxypropyl]phosphonousacid,
(3-aminopropyl)methylphosphinic acid,
(3-amino-2-hydroxypropyl)methylphosphinic acid,
(3-aminopropyl)(difluoromethyl)phosphinic acid,
(4-aminobut-2-yl)methylphosphinic acid,
(3-amino-1-hydroxypropyl)methylphosphinic acid,
(3-amino-2-hydroxypropyl)(difluoromethyl)phosphinic acid,
(E)-(3-aminopropen-1-yl)methylphosphinic acid,
(3-amino-2-oxo-propyl)methyl phosphinic acid,
(3-aminopropyl)hydroxymethylphosphinic acid,
(5-aminopent-3-yl)methylphosphinic acid,
(4-amino-1,1,1-trifluorobut-2-yl)methylphosphinic acid,
(3-amino-2-(4-chlorophenyl)propyl)sulfinic acid,
3-aminopropylsulfinic acid
and the pharmaceutically acceptable hydrates, solvates, salts and stereoisomers thereof.

26. A kit-of-parts comprising a preparation of a first active ingredient, which is an acid pump antagonist, as defined in claim 1, together with a pharmaceutically acceptable carrier or diluent; and a preparation of a second active ingredient, which is a compound which modifies gastrointestinal motility, as defined in claim 1, together with a pharmaceutically acceptable carrier or diluent; and optionally instructions for simultaneous, sequential, separate or chronologically staggered use in therapy.

27. A method to measure compound-associated modulation of the number of transient lower esophageal sphincter relaxations (TLOSRs) comprising the following steps
a.) connecting a gastric fistula dog via the gastric fistula to a barostat which continuously adjusts an elevated gastric target pressure by pumping or sucking a suitable gas mixture containing a suitable detecting gas causing an appropriate number of TLOSRs leading to eructations,
b.) administering one or more compounds optionally sequentially, separately or simultaneously to said dog,
c.) quantificating said TLOSRs via measuring the numbers of said eructations by detecting quantitatively the concentration of detecting gas eructated.

28. The combination according to claim 2, wherein the second active ingredient is selected from the group consisting of TICALOPRIDE, PIBOSEROD, LY-353433, YM-114, CILANSETRON, RAMOSETRON, ALOSETRON, TEGASEROD, PRUCALOPRIDE, FABESETRON, E-3620, RENZAPRIDE, DEXLOXIGLUMIDE, NEPADUTANT, SAREDUTANT, TALNETANT, FEDOTOZINE, PTI-901, ASIMADOLINE, ALVIMOPAN, ZAMIFENACIN, (S)-OXYBUTININ, J-104135, DARIFENAZIN, MOSAPRIDE, PUMOSETRAG, MITEMCINAL, ITRIGLUMIDE, Z-360, LIREXAPRIDE, BIMU-1 and R-137696.

29. The combination according to claim 5, wherein the second active ingredient is selected from the group consisting of TICALOPRIDE, PIBOSEROD, LY-353433, YM-114, CILANSETRON, RAMOSETRON, ALOSETRON, TEGASEROD, PRUCALOPRIDE, FABESETRON, E-3620, RENZAPRIDE, DEXLOXIGLUMIDE, NEPADUTANT, SAREDUTANT, TALNETANT, FEDOTOZINE, PTI-901, ASIMADOLINE, ALVIMOPAN, ZAMIFENACIN, (S)-OXYBUTININ, J-104135, DARIFENAZIN, MOSAPRIDE, PUMOSETRAG, MITEMCINAL, ITRIGLUMIDE, Z-360, LIREXAPRIDE, BIMU-1 and R-137696.

30. The combination according to claim 6, wherein the second active ingredient is selected from the group consisting of TICALOPRIDE, PIBOSEROD, LY-353433, YM-114, CILANSETRON, RAMOSETRON, ALOSETRON, TEGASEROD, PRUCALOPRIDE, FABESETRON, E-3620, RENZAPRIDE, DEXLOXIGLUMIDE, NEPADUTANT, SAREDUTANT, TALNETANT, FEDOTOZINE, PTI-901, ASIMADOLINE, ALVIMOPAN, ZAMIFENACIN, (S)-OXYBUTININ, J-104135 and DARIFENAZIN.

31. The combination according to claim 7, wherein the second active ingredient is selected from the group consisting of TICALOPRIDE, TEGASEROD, PIBOSEROD, MOSAPRIDE, PUMOSETRAG, MITEMCINAL, ITRAGLUMIDE, Z-360 and DEXLOXIGLUMIDE.

32. The combination according to claim 8, wherein the second active ingredient is selected from the group consisting of DOBUPRIDE, KW-5092, KW-5139, R-137696, SR-58611-A, T-1815, Z-338, CINITAPRIDE, ALEMCINAL, IDREMCINAL, MITEMCINAL, SK-896, ITOPRIDE, LEVOSULPIRIDE, METOCLOPRAMIDE, TICALOPRIDE, BIMU-1, CILANSETRON, DAZOPRIDE, E-3620, EM-523, FABESETRON, LINTOPRIDE, LIREXAPRIDE, MOSAPRIDE, PIBOSEROD, PUMOSETRAG, R-137696, RENZAPRIDE, RICASETRON, TICALOPRIDE, Y-36912, YM-114, YM-47813, ZACOPRIDE, TEGASEROD, PRUCALOPRIDE, DARIFENACIN, ASIMADOLINE, FEDOTOZINE, RENZAPRIDE, DEXLOXIGLUMIDE and ITRIGLUMIDE.

33. The combination according to claim 9, wherein the second active ingredient is selected from the group consisting of TEGASEROD, MOSAPRIDE, PRUCALOPRIDE, CISAPRIDE, NOR-CISAPRIDE, (+)-NOR-CISAPRIDE, BIMU1, BIMU8, RENZAPRIDE, ZACOPRIDE, LINTOPRIDE, ITASETRON, FABESETRON, E-3620, CILANSETRON, ALOSETRON, RAMOSETRON, AZASETRON, ONDANSETRON, DOLASETRON, GRANISETRON, TROPISETRON, PIBOSEROD, LY-353433, YM-31636, PUMOSETRAG, ALVIMOPAN and DEXLOXIGLUMIDE.
